(19) United States
(12) Patent Application Publication
(10) Pub. No.: US 2024/0415129 A1
(43) Pub. Date: Dec. 19, 2024

(54) BOTANICAL BASED BIO-INSECTICIDAL COMPOSITIONS FOR CONTROLLING PESTS AND IMPROVING THE PLANT PHYSIOLOGICAL PROCESS

(71) Applicant: KAY BEE BIO ORGANICS PVT LTD, Phaltan (IN)

(72) Inventors: Sachin Babanrao YADAV, Phaltan (IN); Tejas Pandurang JADHAV, Phaltan (IN); Ashant Hanumant SABLE, Phaltan (IN)

(21) Appl. No.: 18/495,111

(22) Filed: Oct. 26, 2023

(30) Foreign Application Priority Data
Jun. 13, 2023 (IN) 202321040275

(51) Int. Cl. A01N 65/22; A01N 25/04; A01N 65/10; A01N 65/18; A01N 65/20; A01N 65/24; A01N 65/26; A01N 65/28; A01N 65/36; A01N 65/38; A01N 65/40; A01P 7/04; A01P 21/00; B82Y 5/00

(52) U.S. Cl. CPC A01N 65/22; A01N 25/04; A01N 65/10; A01N 65/18; A01N 65/20; A01N 65/24; A01N 65/26; A01N 65/28; A01N 65/36; A01N 65/38; A01N 65/40; A01P 7/04; A01P 21/00; B82Y 5/00

(57) ABSTRACT

Disclosed herein is a synergistic botanical based bioinsecticidal, bio-stimulant composition for controlling pests, increasing plant growth and crop yield by improving plant physiological process like photosynthesis and other essential metabolic process in plants/crops that is cost effective and environment friendly. Disclosed herein also is a process for preparation of said composition.

### TECHNICAL FIELD OF THE INVENTION

The present invention relates to a synergistic botanical based bioinsecticidal, bio-stimulant composition for controlling pests, increasing plant growth and crop yield by improving plant physiological process like photosynthesis and other essential metabolic process in plants/crops that is cost effective and environment friendly. The present invention also relates to process for preparation of said composition.

### BACKGROUND OF THE INVENTION

Chemical pesticides play an important role in agricultural production due to their characteristics such as high efficacy, high service efficiency and remarkable economic benefits. However, chemical pesticides cause a number of environmental and social problems, such as, pest resistance to insecticide, pesticide residue, and pest rebirth/resurgence. In addition to these, chemical pesticides are the main cause of wide range toxicity to humans, reflecting in an increase in the number of cancer cases globally. Along with people paying attention to environmental and health issues on a daily basis, using safer bioinsecticides on crops has become a top priority to reduce the use of chemical pesticides.

Synthetic chemical pesticides, if not handled with extreme caution, are highly toxic and deadly to pests and people who are directly or indirectly exposed to them. However, these chemical pesticides are widely used to maximize crop productivity, resulting in negative environmental effects that are harmful to humans and other animals.

Chemical pesticides are effective in controlling or killing crop pests. However, with repeated application, pests develop tolerance and resistance to these chemical pesticides. When chemical pesticides become ineffective, other synthetic chemicals to which pests have not developed resistance are used. This cycle of constantly adding new synthetic chemicals to control pests results in toxic chemicals being released into the environment, which are hazardous to both humans and the environment. The non-selective mode of action of this pesticide also makes it undesirable. Furthermore, these synthetic chemicals can frequently persist in the environment indefinitely.

Despite the fact that it has been over 80 years since the first discovery of a major agricultural pest becoming resistant to a pesticide, it was not until the 1950s that most growers became acquainted with pesticide resistance as a result of the widespread development of insect resistance to DDT, which was later banned due to its severe toxicity to humans. (Reference: ANKERSMIT, GW: “DDT resistance in Plutella maculipennis (Curt.) (Lepidoptera) in Java”, BULL. ENTOMOL. RES., vol. 44, 1953, pages 421-425). Growers have come to expect the eventual loss of pesticide effectiveness due to resistance. By the mid-1980s, there were records of approximately 450 resistant insect and mite species. For any given pest, examples of resistance to chemical insecticides can be found.

As people struggle with environmental and health issues every day, using safer bioinsecticides on crops has become a top priority to reduce the use of chemical pesticides. Bioinsecticides are efficient to use, have low toxicity, leave little residue, and are easy to decompose. The use of botanical pesticides can effectively reduce chemical pesticide consumption; therefore, the research and development of botanical pesticides that use plant extracts and chemical pesticides in combination has become a study hotspot of Pesticide Science in recent years.

The use of natural pesticides is one such alternative that is gaining popularity. Natural pesticides are compounds that occur in nature (plants, microorganisms, etc.) that can be used as such or in combination with synthetic chemical pesticides, or modified so that they can be used as substitutes for synthetic chemical pesticides, or modified along with botanical extracts and essential oils that are eco-friendly, safe for agriculture and humans.

Botanical pesticides are of great interest because they are natural pesticides and the plant-derived phyto-ingredients are safe for humans and the environment. Botanical pesticides are eco-friendly and environmentally safe, as well as cost-effective, target-specific, and biodegradable. A synergistic combination of natural products would typically have no direct toxic effect at the dosage used, and such products will be able to significantly enhance the observed pesticide toxicity when they are combined.

Bioinsecticides are efficient, have low toxicity, leave little residue, and are easy to decompose. The use of plant pesticides can effectively reduce the consumption of chemical pesticides, so the research and development of plant based pesticides and the use of plant extracts have become research hotspots in pesticide science in recent years.

Botanical insecticide or Bio insecticide simply refers to compositions extracted from plants that have insecticidal activity and are studied for their insecticidal activity and mechanism of action. It is possible to manufacture and use insecticidal active ingredients contained in plants as insecticides.

Plant insecticides have unrivalled advantages over chemical pesticides:
1. Plant insecticides do not compromise their natural enemy-producing effectiveness in controlling pests, are relatively safe against non-target organisms, and do not disrupt natural ecological defense systems.
2. The mechanism of action of plant insecticides differs from that of chemical pesticides. Plant insecticide is composed of multi Phyto ingredients so it is very difficult for pest to develop resistance against it and same time different phyto ingrident has different mode of action against pest so its very effective.
3. Plant insecticide are naturally derived, easily degraded, represent the circulation of natural resources, are characterized by low toxicity and low residue, and are relatively safe for humans and other animals.
4. There are about 250,000 plants in the world that are rich in plant insecticide resources, of which more than 4,000 plants have biocidal effects, and their exploitation has great potential.

Some examples of existing prior arts are provided below:

CN 101103733 deals with a plant pesticide emulsifiable solution and aqua made from calamus medicinal plants that solves the problem caused by the widespread use of chemical pesticides in the prevention and treatment of stored grain pests, sanitary insect pests, vegetable insect pests, and flower insect pests.

CN 1810123 describes botanical pesticides such as pesticide oil emulsion and pesticide oil aqua containing calamus extracting concentrate that are used on stored grains, household items, vegetables, and flowers.

U.S. Pat. No. 5,435,821 This patent describes the use of a pesticide as part of a larger system aimed at increasing the growth of vegetable matter.

U.S. Pat. No. 6,855,351 Pesticide formulation containing Azadirachtin (at least 300 PPM) and Salanin in a product formulated with Neem oil. This patent is another example of a pesticide and demonstrates a specific process for producing the specified pesticide.

U.S. Pat. No. 7,029,687 In this patent, sulphur well water is mixed with seven edible ingredients to create a pesticide that is lethal to fire ants and termites.

None of the prior arts above discuss a botanical pesticide that is exclusively for agriculture, horticulture, olericulture, and floriculture crops and is eco-friendly, low in residuals, and non-toxic to mammals. Despite the fact that they are plant based, nothing is said about the standardisation of same. There is no mention of synergistic interactions in their biological activity, formulation manufacturing process, or equipment used to reduce particle size in nano range, no mention of comparative study of botanical base biopesticide with synthetic chemical pesticides in vitro as well as in vivo, and most importantly, none of the patents explain how their bio stimulant activity occur in plants as the ingredients are derived from plants itself.

Therefore, there is a need in the art to provide alternative pesticides that reduce the use of synthetic chemical compounds, are environmentally friendly, and are safe for agriculture and humans.

Hence, the present inventors, with an aim to control the pest and improve plant growth and crop yield have come up with a synergistic bioinsecticidal composition which is eco friendly, relatively inexpensive, bio-degradable, residue free, safe for agriculture and humans and minimizes the risk of resistance development.

### OBJECT OF INVENTION

It is an object of the present invention to provide botanical based bioinsecticides from standardized, concentrated plant extracts or purified plant marker compounds thereby controlling the plant pests/insects and protects agriculture, horticulture, olericulture and floricultural crops.

It is another object of the present invention to provide a process for preparation of said bioinsecticidal composition.

### SUMMARY OF THE INVENTION

In an aspect, the present invention provides a botanical based bio-insecticidal, bio-stimulant composition comprising a combination of standardised enriched botanical extract and purified phyto ingredient with a synergistic biological action that efficiently controls a wide range of pests that cause crop destruction by taking direct action on them.

In a main aspect, the present invention provides a synergistic botanical based bio-insecticidal, bio-stimulant composition for controlling pests, comprising:
i. Thymus vulgaris extract or Enriched and Purified extract of Thymus vulgaris or Purified Phyto ingredients or marker compounds such as 1,8-cineole, camphor, α-pinene, camphene, carvarcrol and thymolin extracted from said botanical source at a concentration in a range of 0.001-35%;
ii. Cinnamomum cassia extract or Enriched and Purified extract of Cinnamomum cassia or Purified Phyto ingredients or marker compounds such as Cinnamic aldehyde, catechin, epicatechin, quercitrin, protocatechuic acid and cinnamic acid extracted from said botanical source at a concentration in a range of 0.001-35%;
iii. Cuminum cyminum extract or Enriched and Purified extract of Cuminum cyminum or Purified Phyto ingredients or marker compounds such as Cumin aldehyde, alkaloid, anthraquinone, saponin, tannin and steroid extracted from said botanical source at a concentration in a range of 0.001-45%;
iv. Abrus precatorius extract or enriched and purified extract of Abrus precatorius or Purified Phyto ingredients or marker compounds such as Abrin and α-tocopherol extracted from said botanical source at a concentration in a range of 0.001-45%;
v. Piper nigrum extract or Enriched and Purified extract of Piper nigrum or Purified Phyto ingredients or marker compounds such as Piperine, amides, piperidine and pyrrolidines extracted from said botanical source at a concentration in a range of 0.001-35%;
vi. Piper longum extract or Enriched and Purified extract of Piper longum or Purified Phyto ingredients or marker compounds such as methyl piperine, Piperine piperlongumine, piperlonguminine, pipercide and piperidine extracted from said botanical source at a concentration in a range of 0.001-45%;
vii. Syzygium aromaticum extract or Enriched and Purified extract of Syzygium aromaticum or Purified Phyto ingredients or marker compounds such as Eugenyl acetate, eugenol, and β-caryophyllene extracted from said botanical source at a concentration in a range of 0.001-45%;
viii. Pongamia pinnata extract or Enriched and Purified extract of Pongamia pinnata or Purified Phyto ingredients or marker compounds such as karanjin, glabrin, and kanjone extracted from said botanical source at a concentration in a range of 0.001-45%;
ix. Anethum graveolens extract or Enriched and Purified extract of Anethum graveolens or Purified Phyto ingredients or marker compounds such as alkaloids, flavonoids, tannins and saponins extracted from said botanical source at a concentration in a range of 0.001-45%;
x. Cinnamomum camphora extract or Enriched and Purified extract of Cinnamomum camphora or Purified Phyto ingredients or marker compounds such as linalool, 1,8-cineole, α-terpineol, and camphor extracted from said botanical source at a concentration in a range of 0.001-45%;
xi. Acorus calamus extract or Enriched and Purified extract of Acorus calamus or Purified Phyto ingredients or marker compounds such as methyl ethers, camphene, P cymene, a selinene, bgurjunene, β cadinene, camphor, shyobunones, linalool and preisocalamendiol extracted from said botanical source at a concentration in a range of 0.001-45%;
xii. Citrus limon extract or Enriched and Purified extract of Citrus limon or Purified Phyto ingredients or marker compounds such as Pinene (β), Limonene, Terpinene (γ), Terpinen-4-ol, Neral Geranial extracted from said botanical source at a concentration in a range of 0.001-45%;
xiii. Azadirachta indica extract or Enriched and Purified extract of Azadirachta indica or Purified Phyto ingredients or marker compounds such as Annonacin, Squamocin, Annonin, kaempferol-3-O and lupeol, β- and α-amyrin, quercetin, camphor, ricin, epicatechin, catechin, linoleic acid and ricinoleic acid extracted from said botanical source at a concentration in a range of 0.001-45%;
xiv. Sesamum indica extract or Enriched and Purified extract of Sesamum indica or Purified Phyto ingredients or marker compounds such as Sesamin, kaempferol-3-O and lupeol, β- and α-amyrin, quercetin, camphor, ricin, epicatechin, catechin, linoleic acid and ricinoleic acid extracted from said botanical source at a concentration in a range of 0.001-55%;
xv. Argemone mexicana extract or Enriched and Purified extract of Argemone Mexicana or Purified Phyto ingredients or marker compounds such as berberine, cryptopine, scoulerine, stylopine, sanguinarine, thalifoline and protopine extracted from said botanical source at a concentration in a range of 0.001-55%; xvi. Moringa oleifera extract or Enriched and Purified extract of Moringa oleifera or Purified Phyto ingredients or marker compounds such as alkaloids, saponins, tannins, steroids, phenolic acids, glucosinolates, flavonoids, and terpenes extracted from said botanical source at a concentration in a range of 0.001-55%; xvii. Brassica juncea extract or Enriched and Purified extract of Brassica juncea or Purified Phyto ingredients or marker compounds such as brassinolide, polyphenols, phenolic acids, flavonoids, carotenoids (zeaxanthin, lutein, β-carotene), alkaloids, tannins and saponins extracted from said botanical source at a concentration in a range of 0.001-55%;
xviii. Lantana camara extract or Enriched and Purified extract of Lantana camara or Purified Phyto ingredients or marker compounds such as tannin, saponin, flavonoids, terpenoids, alkaloids, carbohydrate, anthroquinone and polyphenols extracted from said botanical source at a concentration in a range of 0.001-55%;
xix. Datura stramonium extract or Enriched and Purified extract of Datura stramonium or Purified Phyto ingredients or marker compounds such as Atropine, scopolamine and hyoscyamine extracted from said botanical source at a concentration in a range of 0.001-55%;
xx. Melia dubia extract or Enriched and Purified extract of Melia dubia or Purified Phyto ingredients or marker compounds such as Alkaloids, Carbohydrate, Phenol, Saponin, Terpenoids, Coumarin and Glycoside extracted from said botanical source at a concentration in a range of 0.001-55%;
xxi. Murraya koenigii extract or Enriched and Purified extract of Murraya koenigii or Purified Phyto ingredients or marker compounds such as Alkaloids, Carbohydrate, Phenol, Saponin, Terpenoids, Coumarin and Glycoside extracted from said botanical source at a concentration in a range of 0.001-55%;
xxii. Ipomoea carnea extract or Enriched and Purified extract of Ipomoea carnea or Purified Phyto ingredients or marker compounds such as 2-ethyl-1,3-dimethylbenzene, 2-(12-pentadecynyloxy) tetrahydro2H-pyran, 3-furanyl [2-hydroxy-4-methyl-2-(2-methylpropyl)cyclopentyl]-methanone, 2,2-dideuterooctadecanal and hexadecanoic acid extracted from said botanical source at a concentration in a range of 0.001-55%;
xxiii. Ocimum sanctum extract or Enriched and Purified extract of Ocimum sanctum or Purified Phyto ingredients or marker compounds such as oleanolic acid, rosmarinic acid, ursolic acid, eugenol, methyl eugenol, linalool, carvacrol, β elemene and β caryophyllene extracted from said botanical source at a concentration in a range of 0.001-55%;
xxiv. Jatropha integerrima extract or Enriched and Purified extract of Jatropha integerrima or Purified Phyto ingredients or marker compounds such as Anthocyanin, coumarin, glycoside, phenol, saponin, and tannin extracted from said botanical source at a concentration in a range of 0.001-55%;
xxv. Acacia concinna extract or Enriched and Purified extract of Acacia concinna or Purified Phyto ingredients or marker compounds such as lupeol, spinasterol, acacic acid, lactone and natural sugars glucose extracted from said botanical source at a concentration in a range of 0.001-55%;
xxvi. Mentha piperita extract or Enriched and Purified extract of Mentha piperita or Purified Phyto ingredients or marker compounds such as lupeol, spinasterol, acacic acid, lactone and natural sugars glucose extracted from said botanical source at a concentration in a range of 0.001-55%;
xxvii. Coriandrum sativum extract or Enriched and Purified extract of Coriandrum sativum or Purified Phyto ingredients or marker compounds such as Linalool, neryl acetate, γ-terpinene and α-pinene extracted from said botanical source at a concentration in a range of 0.001-55%;
xxviii. Annona squamosa extract or Enriched and Purified extract of Annona squamosa or Purified Phyto ingredients or marker compounds such as Annonacin, Squamocin, Annonin extracted from said botanical source at a concentration in a range of 0.001-45%;
xxix. organic emulsifier at a concentration in a range of 0.001-20%; preferably 10-12%
xxx. Ionic emulsifier in a range of 0.001-7%, preferably 4-6%,
xxxi. non-ionic emulsifiers in a range of 0.001-10%, preferably 5-7%; and
xxxii. carrier oils at a concentration in a range of 0.001 to 90%, preferably 60-65%.

In an aspect, the synergistic botanical based bio-stimulant, bio-insecticidal composition of the present invention has a particle size in the range of 10-900 nanometer, more specifically 10-100 nanometer.

In another aspect, the synergistic botanical based bio-stimulant, bio-insecticidal composition of the present invention comprising combination of standardised enriched botanical extract and purified phyto ingredient with a synergistic biological action that efficiently controls a wide range of pests that cause crop destruction by taking direct action on them.

In yet another aspect, the synergistic botanical based bio-stimulant, bio-insecticidal composition of the present invention include enriched and purified Phyto ingredients such as saponins, polyphenols, alkaloids, flavonoids, terpenoids, or secondary metabolites and such like from said plants which produce important synergic interactions in their biological activity. The combination of these ingredients provides the desired synergistic benefits and effects to control the pests and provide the simultaneous growth of the plants resulting in high yield of the crops.

In another aspect, the present invention provides a synergistic botanical based bio-insecticidal, bio-stimulant composition, comprising:
i. Melia dubia extract in the range of 0.001 to 35%;
ii. Cinnamomum cassia extract in the range of 0.001 to 35%;
iii. Piper longum extract in the range of 0.001 to 40%;
iv. Lantana camara extract in the range of 0.001 to 25%;
v. Murraya koenigii extract in the range of 0.001 to 35%;
vi. Acorus calamus extract in the range of 0.001 to 35%;
vii. Ipomoea carnea extract in the range of 0.001 to 25%;
viii. Gaur gum in the range of 0.001 to 18%;
ix. ethoxylated castor oil in the range of 0.001 to 20%; and
x. Canola oil in the range of 0.001 to 90%

In yet another aspect, the present invention provides a synergistic botanical based bio-insecticidal, bio-stimulant composition, comprising:
i. Mentha piperita extract in the range of 0.001 to 35%;
ii. Piper nigrum extract in the range of 0.001 to 35%;
iii. Acorus calamus extract in the range of 0.001 to 40%;
iv. Coriandrum sativum extract in the range of 0.001 to 25%;
v. Cinnamomum cassia extract in the range of 0.001 to 35%;
vi. Gaur gum in the range of 0.001 to 18%;
vii. ethoxylated castor oil in the range of 0.001 to 20%; and
viii. Canola oil in the range of 0.001 to 90%

In yet another aspect, the present invention provides a synergistic botanical based bio-insecticidal, bio-stimulant composition, comprising:
i. Piper longum extract in the range of 0.001 to 35%;
ii. Syzygium aromaticum extract in the range of 0.001 to 35%;
iii. Mentha piperita extract in the range of 0.001 to 40%;
iv. Coriandrum sativum extract in the range of 0.001 to 25%;
v. Acorus calamus extract in the range of 0.001 to 35%;
vi. Gaur gum in the range of 0.001 to 18%;
vii. ethoxylated castor oil in the range of 0.001 to 20%; and
viii. Canola oil in the range of 0.001 to 90%

In yet another aspect, the present invention provides a synergistic botanical based bio-insecticidal, bio-stimulant composition, comprising:
i. Argemone mexicana extract in the range of 0.001 to 35%;
ii. Datura stramonium extract in the range of 0.001 to 35%;
iii. Piper longum extract in the range of 0.001 to 40%;
iv. Lantana camara extract in the range of 0.001 to 25%;
v. Brassica juncea extract in the range of 0.001 to 35%;
vi. Acorus calamus extract in the range of 0.001 to 35%;
vii. Ipomoea carnea extract in the range of 0.001 to 25%;
viii. Gaur gum in the range of 0.001 to 18%;
ix. ethoxylated castor oil in the range of 0.001 to 20%; and
x. Canola oil in the range of 0.001 to 90%

In yet another aspect, the present invention provides a synergistic botanical based bio-insecticidal, bio-stimulant composition, comprising:
i. Lantana camara extract in the range of 0.001 to 35%;
ii. Piper nigrum extract in the range of 0.001 to 35%;
iii. Cinnamomum camphora extract in the range of 0.001 to 40%;
iv. Piper longum extract in the range of 0.001 to 25%;
v. Ocimum santum extract in the range of 0.001 to 35%;
vi. Thymus vulgaris extract in the range of 0.001 to 35%;
vii. Citrus limon extract in the range of 0.001 to 25%;
viii. Gaur gum in the range of 0.001 to 18%;
ix. ethoxylated castor oil in the range of 0.001 to 20%; and
x. Canola oil in the range of 0.001 to 90%

In yet another aspect, the present invention provides a synergistic botanical based bio-insecticidal, bio-stimulant composition, comprising:
i. Abrus precatorius extract in the range of 0.001 to 35%;
ii. Acorus calamus extract in the range of 0.001 to 35%;
iii. Jatropha integerrima extract in the range of 0.001 to 40%;
iv. Piper longum extract in the range of 0.001 to 25%;
v. Acacia concinna extract in the range of 0.001 to 35%;
vi. Cuminum cyminum extract in the range of 0.001 to 35%;
vii. Gaur gum in the range of 0.001 to 18%;
viii. ethoxylated castor oil in the range of 0.001 to 20%; and

ix. Canola oil in the range of 0.001 to 90%

In yet another aspect, the present invention provides a process for preparation of the synergistic botanical based bio-insecticidal, bio-stimulant composition, comprising:
a. Extracting 0.001-35% of Thymus vulgaris or purified phyto ingredients or marker compounds such as 1,8-cineole, camphor, α-pinene, camphene, carvarcrol and thymolin extracted from said botanical source; 0.001-35% of Cinnamomum cassia or Purified Phyto ingredients or marker compounds such as Cinnamic aldehyde, catechin, epicatechin, quercitrin, protocatechuic acid and cinnamic acid extracted from said botanical source; 0.001-45% of Cuminum cyminum or Purified Phyto ingredients or marker compounds such as Cumin aldehyde, alkaloid, anthraquinone, saponin, tannin and steroid extracted from said botanical source; 0.001-45% of Abrus precatorius or Purified Phyto ingredients or marker compounds such as Abrin and α-tocopherol extracted from said botanical source; 0.001-35% of Piper nigrum or Purified Phyto ingredients or marker compounds such as Piperine, amides, piperidine and pyrrolidines extracted from said botanical source; 0.001-45% of Piper longum or Purified Phyto ingredients or marker compounds such as methyl piperine, Piperine piperlongumine, piperlonguminine, pipercide and piperidine extracted from said botanical source; 0.001-45% of Syzygium aromaticum or Purified Phyto ingredients or marker compounds such as Eugenyl acetate, eugenol, and β-caryophyllene extracted from said botanical source; 0.001-45% of Pongamia pinnata Purified Phyto ingredients or marker compounds such as karanjin, glabrin, and kanjone extracted from said botanical source; 0.001-45% of Anethum graveolens or Purified Phyto ingredients or marker compounds such as alkaloids, flavonoids, tannins and saponins extracted from said botanical source; 0.001-45% of Cinnamomum camphora or Purified Phyto ingredients or marker compounds such as linalool, 1,8-cineole, α-terpineol, and camphor extracted from said botanical source; 0.001-45% of Acorus calamus or Purified Phyto ingredients or marker compounds such as methyl ethers, camphene, P cymene, α selinene, bgurjunene, β cadinene, camphor, shyobunones, linalool and preisocalamendiol extracted from said botanical source; 0.001-45% of Citrus limon or Purified Phyto ingredients or marker compounds such as Pinene (β), Limonene, Terpinene (γ), Terpinen-4-ol, Neral Geranial extracted from said botanical source; 0.001-45% of Azadirachta indica or Purified Phyto ingredients or marker compounds such as Annonacin, Squamocin, Annonin, kaempferol-3-O and lupeol, β- and α-amyrin, quercetin, camphor, ricin, epicatechin, catechin, linoleic acid and ricinoleic acid extracted from said botanical source; 0.001-55% of Sesamum indica or Purified Phyto ingredients or marker compounds such as Sesamin, kaempferol-3-O and lupeol, β- and α-amyrin, quercetin, camphor, ricin, epicatechin, catechin, linoleic acid and ricinoleic acid extracted from said botanical source; 0.001-55% of Argemone Mexicana or Purified Phyto ingredients or marker compounds such as berberine, cryptopine, scoulerine, stylopine, sanguinarine, thalifoline and protopine extracted from said botanical source; 0.001-55% of Moringa oleifera or Purified Phyto ingredients or marker compounds such as alkaloids, saponins, tannins, steroids, phenolic acids, glucosinolates, flavonoids, and terpenes extracted from said botanical source; 0.001-55% of Brassica juncea or Purified Phyto ingredients or marker compounds such as brassinolide, polyphenols, phenolic acids, flavonoids, carotenoids (zeaxanthin, lutein, β-carotene), alkaloids, tannins and saponins extracted from said botanical source; 0.001-55% of Lantana camara or Purified Phyto ingredients or marker compounds such as tannin, saponin, flavonoids, terpenoids, alkaloids, carbohydrate, anthroquinone and polyphenols extracted from said botanical source; 0.001-55% of Datura stramonium or Purified Phyto ingredients or marker compounds such as Atropine, scopolamine and hyoscyamine extracted from said botanical source; 0.001-55% of Melia dubia or Purified Phyto ingredients or marker compounds such as Alkaloids, Carbohydrate, Phenol, Saponin, Terpenoids, Coumarin and Glycoside extracted from said botanical source; 0.001-55% of Murraya koenigii or Purified Phyto ingredients or marker compounds such as Alkaloids, Carbohydrate, Phenol, Saponin, Terpenoids, Coumarin and Glycoside extracted from said botanical source; 0.001-55% of Ipomoea carnea or Purified Phyto ingredients or marker compounds such as 2-ethyl-1,3-dimethylbenzene, 2-(12-pentadecynyloxy) tetrahydro2H-pyran, 3-furanyl [2-hydroxy-4-methyl-2-(2-methylpropyl)cyclopentyl]-methanone, 2,2-dideuterooctadecanal and hexadecanoic acid extracted from said botanical source; 0.001-55% of Ocimum sanctum or Purified Phyto ingredients or marker compounds such as oleanolic acid, rosmarinic acid, ursolic acid, eugenol, methyl eugenol, linalool, carvacrol, β elemene and β caryophyllene extracted from said botanical source; 0.001-55% of Jatropha integerrima or Purified Phyto ingredients or marker compounds such as Anthocyanin, coumarin, glycoside, phenol, saponin, and tannin extracted from said botanical source; 0.001-55 of Acacia concinna or Purified Phyto ingredients or marker compounds such as lupeol, spinasterol, acacic acid, lactone and natural sugars glucose extracted from said botanical source; 0.001-55% of Mentha piperita or Purified Phyto ingredients or marker compounds such as lupeol, spinasterol, acacic acid, lactone and natural sugars glucose extracted from said botanical source; 0.001-55% of Coriandrum sativum or Purified Phyto ingredients or marker compounds such as Linalool, neryl acetate, γ-terpinene and α-pinene extracted from said botanical source; 0.001-45% of Annona squamosa or Purified Phyto ingredients or marker compounds such as Annonacin, Squamocin, Annonin extracted from said botanical source;
b. mixing the obtained extracts at given proportion along with 0.001-20% of organic emulsifier, preferably 10-12%; 0.001-7% of Ionic, preferably 4-6%; and 0.001-10% of non-ionic emulsifiers, preferably 5-7%;
c. blending the mixture obtained in step (b) with 0.001 to 90% of carrier oils, preferably 60-65%;
d. stirring the mixture obtained in step (c) at 300-1000 RPM, preferably 500-800 RPM at 25 to 55° C., preferably 35° C. in close mixing vessel for continued stirring followed by homogenizing the mix with the homogenizer fitted towards the bottom of vessel and with the speed of 3700˜27000 rpm to reduce the particle size;
e. passing the homogenized mixture obtained in step (d) through in line shear pump with 1800 RPM to 5800 RPM to reduce particle size to obtain the nano emulsion with particle size ranging from 10 to 900 nano meter;
f. passing the mixture obtained in step (e) through High-pressure homogenization to reduce the particle size below 100 nano meters;
g. passing the mixture obtained in step (f) through sparkler filter having seven layers of membrane filters with pore size less than 1-2 micron to achieve desired filtration;
   and
h. recovering the final product.

The composition of the present invention is useful for controlling pests and improving yield of crop plants.

### BRIEF DESCRIPTION OF THE DRAWINGS

FIG. 1: Effect of Bio-stimulants on Curd Yield of Cauliflower.

FIG. 2: Effect of Bio-stimulants on Grain Yield of Chickpea

FIG. 3: Effect of Bio-stimulants on Yield of Gerbera

FIG. 4: Effect of Bio-stimulants on Seed Cotton Yield

FIG. 5: Effect of Bio-stimulants on Yield of Guava

FIG. 6: Effect of Bio-stimulants on Fruit Yield of Tomato

FIG. 7: Effect of Bio-stimulants on Grain Yield of Maize.

FIG. 8: Effect of Bio-stimulants on Yield of Mustard.

FIG. 9: Effect of Bio-stimulants on Flower Yield of Rose

FIG. 10: Effect of Bio-stimulants on Number of Flower of Carnation

FIG. 11: Effect of Bio-stimulants on Grain Yield of Maize

FIG. 12: Effect of Bio-stimulants on Bulb Yield of Onion.

FIG. 13: Percentage reduction in DMP population and Percentage increase in yield

FIG. 14: Percentage reduction in Mealy Bug Population and Percentage increase in yield

FIG. 15: Percentage reduction in Thrips population and Percentage increase in yield

FIG. 16: Percentage Mortality of Diamond Back Moth (DBM) population

FIG. 17: Percentage Mortality of Mealy bug population

FIG. 18: Percentage Mortality of Thrips population

### DETAILED DESCRIPTION OF THE INVENTION

The invention will now be described in detail in connection with certain preferred and optional embodiments, so that various aspects thereof may be more fully understood and appreciated.

In an embodiment, the present invention relates to a synergistic botanical based bio-insecticidal, bio-stimulant composition for controlling pests, comprising:
i. Thymus vulgaris extract or Enriched and Purified extract of Thymus vulgaris or Purified Phyto ingredients or marker compounds such as 1,8-cineole, camphor, α-pinene, camphene, carvarcrol and thymolin extracted from said botanical source at a concentration in a range of 0.001-35%;
ii. Cinnamomum cassia extract or Enriched and Purified extract of Cinnamomum cassia or Purified Phyto ingredients or marker compounds such as Cinnamic aldehyde, catechin, epicatechin, quercitrin, protocatechuic acid and cinnamic acid extracted from said botanical source at a concentration in a range of 0.001-35%; iii. Cuminum cyminum extract or Enriched and Purified extract of Cuminum cyminum or Purified Phyto ingredients or marker compounds such as Cumin aldehyde, alkaloid, anthraquinone, saponin, tannin and steroid extracted from said botanical source at a concentration in a range of 0.001-45%;
iv. Abrus precatorius extract or enriched and purified extract of Abrus precatorius or Purified Phyto ingredients or marker compounds such as Abrin and α-tocopherol extracted from said botanical source at a concentration in a range of 0.001-45%; v. Piper nigrum extract or Enriched and Purified extract of Piper nigrum or Purified Phyto ingredients or marker compounds such as Piperine, amides, piperidine and pyrrolidines extracted from said botanical source at a concentration in a range of 0.001-35%;
vi. Piper longum extract or Enriched and Purified extract of Piper longum or Purified Phyto ingredients or marker compounds such as methyl piperine, Piperine piperlongumine, piperlonguminine, pipercide and piperidine extracted from said botanical source at a concentration in a range of 0.001-45%;
vii. Syzygium aromaticum extract or Enriched and Purified extract of Syzygium aromaticum or Purified Phyto ingredients or marker compounds such as Eugenyl acetate, eugenol, and β-caryophyllene extracted from said botanical source at a concentration in a range of 0.001-45%;
viii. Pongamia pinnata extract or Enriched and Purified extract of Pongamia pinnata or Purified Phyto ingredients or marker compounds such as karanjin, glabrin, and kanjone extracted from said botanical source at a concentration in a range of 0.001-45%;
ix. Anethum graveolens extract or Enriched and Purified extract of Anethum graveolens or Purified Phyto ingredients or marker compounds such as alkaloids, flavonoids, tannins and saponins extracted from said botanical source at a concentration in a range of 0.001-45%;
x. Cinnamomum camphora extract or Enriched and Purified extract of Cinnamomum camphora or Purified Phyto ingredients or marker compounds such as linalool, 1,8-cineole, α-terpineol, and camphor extracted from said botanical source at a concentration in a range of 0.001-45%;
xi. Acorus calamus extract or Enriched and Purified extract of Acorus calamus or Purified Phyto ingredients or marker compounds such as methyl ethers, camphene, P cymene, a selinene, bgurjunene, β cadinene, camphor, shyobunones, linalool and preisocalamendiol extracted from said botanical source at a concentration in a range of 0.001-45%;
xii. Citrus limon extract or Enriched and Purified extract of Citrus limon or Purified Phyto ingredients or marker compounds such as Pinene (β), Limonene, Terpinene (γ), Terpinen-4-ol, Neral Geranial extracted from said botanical source at a concentration in a range of 0.001-45%;
xiii. Azadirachta indica extract or Enriched and Purified extract of Azadirachta indica or Purified Phyto ingredients or marker compounds such as Annonacin, Squamocin, Annonin, kaempferol-3-O and lupeol, β- and α-amyrin, quercetin, camphor, ricin, epicatechin, catechin, linoleic acid and ricinoleic acid extracted from said botanical source at a concentration in a range of 0.001-45%;
xiv. Sesamum indica extract or Enriched and Purified extract of Sesamum indica or Purified Phyto ingredients or marker compounds such as Sesamin, kaempferol-3-O and lupeol, β- and α-amyrin, quercetin, camphor, ricin, epicatechin, catechin, linoleic acid and ricinoleic acid extracted from said botanical source at a concentration in a range of 0.001-55%;
xv. Argemone mexicana extract or Enriched and Purified extract of Argemone Mexicana or Purified Phyto ingredients or marker compounds such as berberine, cryptopine, scoulerine, stylopine, sanguinarine, thalifoline and protopine extracted from said botanical source at a concentration in a range of 0.001-55%; xvi. Moringa oleifera extract or Enriched and Purified extract of Moringa oleifera or Purified Phyto ingredients or marker compounds such as alkaloids, saponins, tannins, steroids, phenolic acids, glucosinolates, flavonoids, and terpenes extracted from said botanical source at a concentration in a range of 0.001-55%; xvii. Brassica juncea extract or Enriched and Purified extract of Brassica juncea or Purified Phyto ingredients or marker compounds such as brassinolide, polyphenols, phenolic acids, flavonoids, carotenoids (zeaxanthin, lutein, β-carotene), alkaloids, tannins and saponins extracted from said botanical source at a concentration in a range of 0.001-55%;
xviii. Lantana camara extract or Enriched and Purified extract of Lantana camara or Purified Phyto ingredients or marker compounds such as tannin, saponin, flavonoids, terpenoids, alkaloids, carbohydrate, anthroquinone and polyphenols extracted from said botanical source at a concentration in a range of 0.001-55%;
xix. Datura stramonium extract or Enriched and Purified extract of Datura stramonium or Purified Phyto ingredients or marker compounds such as Atropine, scopolamine and hyoscyamine extracted from said botanical source at a concentration in a range of 0.001-55%;
xx. Melia dubia extract or Enriched and Purified extract of Melia dubia or Purified Phyto ingredients or marker compounds such as Alkaloids, Carbohydrate, Phenol, Saponin, Terpenoids, Coumarin and Glycoside extracted from said botanical source at a concentration in a range of 0.001-55%;
xxi. Murraya koenigii extract or Enriched and Purified extract of Murraya koenigii or Purified Phyto ingredients or marker compounds such as Alkaloids, Carbohydrate, Phenol, Saponin, Terpenoids, Coumarin and Glycoside extracted from said botanical source at a concentration in a range of 0.001-55%;
xxii. Ipomoea carnea extract or Enriched and Purified extract of Ipomoea carnea or Purified Phyto ingredients or marker compounds such as 2-ethyl-1,3-dimethylbenzene, 2-(12-pentadecynyloxy) tetrahydro2H-pyran, 3-furanyl [2-hydroxy-4-methyl-2-(2-methylpropyl)cyclopentyl]-methanone, 2,2-dideuterooctadecanal and hexadecanoic acid extracted from said botanical source at a concentration in a range of 0.001-55%;
xxiii. Ocimum sanctum extract or Enriched and Purified extract of Ocimum sanctum or Purified Phyto ingredients or marker compounds such as oleanolic acid, rosmarinic acid, ursolic acid, eugenol, methyl eugenol, linalool, carvacrol, β elemene and β caryophyllene extracted from said botanical source at a concentration in a range of 0.001-55%;
xxiv. Jatropha integerrima extract or Enriched and Purified extract of Jatropha integerrima or Purified Phyto ingredients or marker compounds such as Anthocyanin, coumarin, glycoside, phenol, saponin, and tannin extracted from said botanical source at a concentration in a range of 0.001-55%;
xxv. Acacia concinna extract or Enriched and Purified extract of Acacia concinna or Purified Phyto ingredients or marker compounds such as lupeol, spinasterol, acacic acid, lactone and natural sugars glucose extracted from said botanical source at a concentration in a range of 0.001-55%;
xxvi. Mentha piperita extract or Enriched and Purified extract of Mentha piperita or Purified Phyto ingredients or marker compounds such as lupeol, spinasterol, acacic acid, lactone and natural sugars glucose extracted from said botanical source at a concentration in a range of 0.001-55%;
xxvii. Coriandrum sativum extract or Enriched and Purified extract of Coriandrum sativum or Purified Phyto ingredients or marker compounds such as Linalool, neryl acetate, γ-terpinene and α-pinene extracted from said botanical source at a concentration in a range of 0.001-55%;
xxviii. Annona squamosa extract or Enriched and Purified extract of Annona squamosa or Purified Phyto ingredients or marker compounds such as Annonacin, Squamocin, Annonin extracted from said botanical source at a concentration in a range of 0.001-45%;
xxix. organic emulsifier at a concentration in a range of 0.001-20%; preferably 10-12%
xxx. Ionic emulsifier at a concentration in a range of 0.001-7%, preferably 4-6%,
xxxi. non-ionic emulsifiers at a concentration in a range of 0.001-10%, preferably 5-7%; and
xxxii. carrier oils at a concentration in a range of 0.001 to 90%, preferably 60-65%.

In another embodiment, the organic emulsifier include but not limited to rice bran wax, cetearyl glucoside, aloevera extract, plant saponins, aqueous extract of Indian soapberry (sapindus mukorassi), aqueous extract of Acacia concinna, hydrolyzed coconut oil, bee wax, gaur gum and the like.

In another embodiment, the Ionic and non-ionic emulsifier include but not limited to aqueous extract of Indian soapberry (sapindus mukorassi), aqueous extract of Acacia concinna, hydrolyzed coconut oil, bee wax, gaur gum gaur gum, poly sorbate 20, poly sorbate 60, poly sorbate 80, Lecithin, soy lecithin, sodium phosphates, monoglycerides, diglycerides, sodium stearoyl lactylate, glyceryl oleate, ethoxylated oleate, ethoxylated soybean oil, ethoxylated castor oil and the like.

In another embodiment, the carrier oils are selected from the group comprising seed oil of Anethum graveolens, Pongamaia pinnata oil, neem oil, castor oil, fish oil, sesame oil, mustard oil, canola oil, or water.

In another embodiment, the present invention provides a synergistic botanical based bio-insecticidal, bio-stimulant composition, comprising:
i. Melia dubia extract in the range of 0.001 to 35%;
ii. Cinnamomum cassia extract in the range of 0.001 to 35%;
iii. Piper longum extract in the range of 0.001 to 40%;
iv. Lantana camara extract in the range of 0.001 to 25%;
v. Murraya koenigii extract in the range of 0.001 to 35%;
vi. Acorus calamus extract in the range of 0.001 to 35%;
vii. Ipomoea carnea extract in the range of 0.001 to 25%;
viii. Gaur gum in the range of 0.001 to 18%;
ix. ethoxylated castor oil in the range of 0.001 to 20%; and
x. Canola oil in the range of 0.001 to 90%

In yet another embodiment, the present invention provides a synergistic botanical based bio-insecticidal, bio-stimulant composition, comprising:
i. Mentha piperita extract in the range of 0.001 to 35%;
ii. Piper nigrum extract in the range of 0.001 to 35%;
iii. Acorus calamus extract in the range of 0.001 to 40%;
iv. Coriandrum sativum extract in the range of 0.001 to 25%;
v. Cinnamomum cassia extract in the range of 0.001 to 35%;
vi. Gaur gum in the range of 0.001 to 18%;
vii. ethoxylated castor oil in the range of 0.001 to 20%; and
viii. Canola oil in the range of 0.001 to 90%

In yet another embodiment, the present invention provides a synergistic botanical based bio-insecticidal, bio-stimulant composition, comprising:
i. Piper longum extract in the range of 0.001 to 35%;
ii. Syzygium aromaticum extract in the range of 0.001 to 35%;
iii. Mentha piperita extract in the range of 0.001 to 40%;
iv. Coriandrum sativum extract in the range of 0.001 to 25%;
v. Acorus calamus extract in the range of 0.001 to 35%;
vi. Gaur gum in the range of 0.001 to 18%;
vii. ethoxylated castor oil in the range of 0.001 to 20%; and
viii. Canola oil in the range of 0.001 to 90%

In yet another embodiment, the present invention provides a synergistic botanical based bio-insecticidal, bio-stimulant composition, comprising:
i. Argemone mexicana extract in the range of 0.001 to 35%;
ii. Datura stramonium extract in the range of 0.001 to 35%;
iii. Piper longum extract in the range of 0.001 to 40%;
iv. Lantana camara extract in the range of 0.001 to 25%;
v. Brassica juncea extract in the range of 0.001 to 35%;
vi. Acorus calamus extract in the range of 0.001 to 35%;
vii. Ipomoea carnea extract in the range of 0.001 to 25%;
viii. Gaur gum in the range of 0.001 to 18%;
ix. ethoxylated castor oil in the range of 0.001 to 20%; and
x. Canola oil in the range of 0.001 to 90%

In yet another embodiment, the present invention provides a synergistic botanical based bio-insecticidal, bio-stimulant composition, comprising:
i. Lantana camara extract in the range of 0.001 to 35%;
ii. Piper nigrum extract in the range of 0.001 to 35%;
iii. Cinnamomum camphora extract in the range of 0.001 to 40%;
iv. Piper longum extract in the range of 0.001 to 25%;
v. Ocimum santum extract in the range of 0.001 to 35%;
vi. Thymus vulgaris extract in the range of 0.001 to 35%;
vii. Citrus limon extract in the range of 0.001 to 25%;
viii. Gaur gum in the range of 0.001 to 18%;
ix. ethoxylated castor oil in the range of 0.001 to 20%; and
x. Canola oil in the range of 0.001 to 90%

In yet another embodiment, the present invention provides a synergistic botanical based bio-insecticidal, bio-stimulant composition, comprising:
i. Abrus precatorius extract in the range of 0.001 to 35%;
ii. Acorus calamus extract in the range of 0.001 to 35%;
iii. Jatropha integerrima extract in the range of 0.001 to 40%;
iv. Piper longum extract in the range of 0.001 to 25%;
v. Acacia concinna extract in the range of 0.001 to 35%;
vi. Cuminum cyminum extract in the range of 0.001 to 35%;
vii. Gaur gum in the range of 0.001 to 18%;
viii. ethoxylated castor oil in the range of 0.001 to 20%; and
ix. Canola oil in the range of 0.001 to 90%

In an embodiment, the synergistic botanical based bio-stimulant, bio-insecticidal composition of the present invention has a particle size in the range of 10-900 nanometer, more specifically 10-100 nanometer.

In another embodiment, the synergistic botanical based bio-stimulant, bio-insecticidal composition of the present invention comprising combination of standardised enriched botanical extract and purified phyto ingredient with a synergistic biological action that efficiently controls a wide range of pests that cause crop destruction by taking direct action on them.

In yet another aspect, the synergistic botanical based bio-stimulant, bio-insecticidal composition of the present invention include enriched and purified Phyto ingredients such as saponins, polyphenols, alkaloids, flavonoids, terpenoids, or secondary metabolites and such like from said plants which produce important synergic interactions in their biological activity. The combination of these ingredients provide the desired synergistic benefits and effects to control the pests and provide the simultaneous growth of the plants resulting in high yield of the crops,

In another embodiment, the synergistic bio-insecticidal, bio-stimulant composition of the present invention is effective against major agricultural pests such as non-flying insects, caterpillar, larvae, sucking pest likes white fly, hopper, Jassid, mealy bugs, aphids, spider mites, slugs, thrips and the like.

In yet another embodiment, the present invention provides a process for preparation of the synergistic botanical based bio-insecticidal, bio-stimulant composition, comprising:
a. Extracting 0.001-35% of Thymus vulgaris or purified phyto ingredients or marker compounds such as 1,8-cineole, camphor, α-pinene, camphene, carvarcrol and thymolin extracted from said botanical source; 0.001-35% of Cinnamomum cassia or Purified Phyto ingredients or marker compounds such as Cinnamic aldehyde, catechin, epicatechin, quercitrin, protocatechuic acid and cinnamic acid extracted from said botanical source; 0.001-45% of Cuminum cyminum or Purified Phyto ingredients or marker compounds such as Cumin aldehyde, alkaloid, anthraquinone, saponin, tannin and steroid extracted from said botanical source; 0.001-45% of Abrus precatorius or Purified Phyto ingredients or marker compounds such as Abrin and α-tocopherol extracted from said botanical source; 0.001-35% of Piper nigrum or Purified Phyto ingredients or marker compounds such as Piperine, amides, piperidine and pyrrolidines extracted from said botanical source; 0.001-45% of Piper longum or Purified Phyto ingredients or marker compounds such as methyl piperine, Piperine piperlongumine, piperlonguminine, pipercide and piperidine extracted from said botanical source; 0.001-45% of Syzygium aromaticum or Purified Phyto ingredients or marker compounds such as Eugenyl acetate, eugenol, and β-caryophyllene extracted from said botanical source; 0.001-45% of Pongamia pinnata Purified Phyto ingredients or marker compounds such as karanjin, glabrin, and kanjone extracted from said botanical source; 0.001-45% of Anethum graveolens or Purified Phyto ingredients or marker compounds such as alkaloids, flavonoids, tannins and saponins extracted from said botanical source; 0.001-45% of Cinnamomum camphora or Purified Phyto ingredients or marker compounds such as linalool, 1,8-cineole, α-terpineol, and camphor extracted from said botanical source; 0.001-45% of Acorus calamus or Purified Phyto ingredients or marker compounds such as methyl ethers, camphene, P cymene, a selinene, bgurjunene, β cadinene, camphor, shyobunones, linalool and preisocalamendiol extracted from said botanical source; 0.001-45% of Citrus limon or Purified Phyto ingredients or marker compounds such as Pinene (β), Limonene, Terpinene (γ), Terpinen-4-ol, Neral Geranial extracted from said botanical source; 0.001-45% of Azadirachta indica or Purified Phyto ingredients or marker compounds such as Annonacin, Squamocin, Annonin, kaempferol-3-O and lupeol, β- and α-amyrin, quercetin, camphor, ricin, epicatechin, catechin, linoleic acid and ricinoleic acid extracted from said botanical source; 0.001-55% of Sesamum indica or Purified Phyto ingredients or marker compounds such as Sesamin, kaempferol-3-O and lupeol, β- and α-amyrin, quercetin, camphor, ricin, epicatechin, catechin, linoleic acid and ricinoleic acid extracted from said botanical source; 0.001-55% of Argemone Mexicana or Purified Phyto ingredients or marker compounds such as berberine, cryptopine, scoulerine, stylopine, sanguinarine, thalifoline and protopine extracted from said botanical source; 0.001-55% of Moringa oleifera or Purified Phyto ingredients or marker compounds such as alkaloids, saponins, tannins, steroids, phenolic acids, glucosinolates, flavonoids, and terpenes extracted from said botanical source; 0.001-55% of Brassica juncea or Purified Phyto ingredients or marker compounds such as brassinolide, polyphenols, phenolic acids, flavonoids, carotenoids (zeaxanthin, lutein, β-carotene), alkaloids, tannins and saponins extracted from said botanical source; 0.001-55% of Lantana camara or Purified Phyto ingredients or marker compounds such as tannin, saponin, flavonoids, terpenoids, alkaloids, carbohydrate, anthroquinone and polyphenols extracted from said botanical source; 0.001-55% of Datura stramonium or Purified Phyto ingredients or marker compounds such as Atropine, scopolamine and hyoscyamine extracted from said botanical source; 0.001-55% of Melia dubia or Purified Phyto ingredients or marker compounds such as Alkaloids, Carbohydrate, Phenol, Saponin, Terpenoids, Coumarin and Glycoside extracted from said botanical source; 0.001-55% of Murraya koenigii or Purified Phyto ingredients or marker compounds such as Alkaloids, Carbohydrate, Phenol, Saponin, Terpenoids, Coumarin and Glycoside extracted from said botanical source; 0.001-55% of Ipomoea carnea or Purified Phyto ingredients or marker compounds such as 2-ethyl-1,3-dimethylbenzene, 2-(12-pentadecynyloxy) tetrahydro2H-pyran, 3-furanyl [2-hydroxy-4-methyl-2-(2-methylpropyl)cyclopentyl]-methanone, 2,2-dideuterooctadecanal and hexadecanoic acid extracted from said botanical source; 0.001-55% of Ocimum sanctum or Purified Phyto ingredients or marker compounds such as oleanolic acid, rosmarinic acid, ursolic acid, eugenol, methyl eugenol, linalool, carvacrol, β elemene and β caryophyllene extracted from said botanical source; 0.001-55% of Jatropha integerrima or Purified Phyto ingredients or marker compounds such as Anthocyanin, coumarin, glycoside, phenol, saponin, and tannin extracted from said botanical source; 0.001-55 of Acacia concinna or Purified Phyto ingredients or marker compounds such as lupeol, spinasterol, acacic acid, lactone and natural sugars glucose extracted from said botanical source; 0.001-55% of Mentha piperita or Purified Phyto ingredients or marker compounds such as lupeol, spinasterol, acacic acid, lactone and natural sugars glucose extracted from said botanical source; 0.001-55% of Coriandrum sativum or Purified Phyto ingredients or marker compounds such as Linalool, neryl acetate, γ-terpinene and α-pinene extracted from said botanical source; 0.001-45% of Annona squamosa or Purified Phyto ingredients or marker compounds such as Annonacin, Squamocin, Annonin extracted from botanical source;
b. mixing the obtained extracts at given proportion along with 0.001-20% of organic emulsifier, preferably 10-12%; 0.001-7% of Ionic, preferably 4-6%; and 0.001-10% % of non-ionic emulsifiers, preferably 5-7%;
c. blending the mixture obtained in step (b) with 0.001 to 90% of carrier oils, preferably 60-65%;
d. stirring the mixture obtained in step (c) at 300-1000 RPM, preferably 500-800 RPM at 25 to 55° C., preferably 35° C. in close mixing vessel for continued stirring stirring followed by homogenizing the mix with the homogenizer fitted towards the bottom of vessel and with the speed of 3700˜27000 rpm to reduce the particle size;
e. passing the homogenized mixture obtained in step (d) through in line shear pump with 1800 RPM to 5800 RPM to reduce particle size to obtain the nano emulsion with particle size ranging from 10 to 900 nano meter;
f. passing the mixture obtained in step (e) through High-pressure homogenization to reduce the particle size below 100 nano meters;
g. passing the mixture obtained in step (f) through sparkler filter having seven layers of membrane filters with pore size less than 1-2 micron to achieve desired filtration; and
h. recovering the final product.

In another embodiment, the mixing vessel in step (d) made up of stainless steel 316 grade with outer jacket for maintaining temperature with a stirrer such as blade stirrer, propeller stirrer, turbine stirrer, anchor stirrer, universal stirrer.

In another embodiment, the homogenization of step (d) is done under very high pressures, sheer, turbulence, acceleration and impact, to make the particles more stable and effective.

In another embodiment, the high-pressure homogenization of step (f) is done at speed of 4000 rpm to 10000 rpm and pressure max up to 4,200 bar (60,000 psi).

In another embodiment, the extraction techniques include but not limited to hydrophilic extraction, super critical carbon dioxide extraction, steam distillation, solvent extraction and Soxhlet extraction, preferably Solvent extraction or super critical carbon dioxide extraction.

In another embodiment, the solvent used in the solvent extraction includes but not limited to methanol, hexane, acetone, ethyl acetate, butanol, or ethanol preferably methanol, ethyl acetate, hexane alone or mixtures thereof.

In another embodiment, the enrichment and purification of the extract using various technique include but not limited to liquid-liquid extraction based on polarity of phyto ingredient, column chromatography by using various medium and matrix, precipitation and crystallisation etc. to obtain purified phyto ingredients.

In another embodiment, the organic emulsifier include but not limited to rice bran wax, cetearyl glucoside, aloevera extract, plant saponins, aqueous extract of Indian soapberry (sapindus mukorassi), aqueous extract of Acacia concinna, hydrolyzed coconut oil, bee wax, gaur gum and the like.

In another embodiment, the Ionic and non-ionic emulsifier include but not limited to, aqueous extract of Indian soapberry (sapindus mukorassi), aqueous extract of Acacia concinna, hydrolyzed coconut oil, bee wax, gaur gum gaur gum, poly sorbate 20, poly sorbate 60, poly sorbate 80, Lecithin, soy lecithin, sodium phosphates, monoglycerides, diglycerides, sodium stearoyl lactylate, glyceryl oleate, ethoxylated oleate, ethoxylated soybean oil, ethoxylated castor oil and the like.

In another embodiment, the synergistic bio-insecticidal, bio-stimulant composition of the present invention may be in the form of liquid, paste, powder or powder converted into granule or tablet form and the like, more preferably in liquid form.

In another embodiment, the synergistic bio-insecticidal, bio-stimulant composition of the present invention can be applied by sprinkler, sprayer or by drip application. The composition may preferably be applied by sprayer application such as foliar sprays by spray pump knapsack sprayer, to plant parts.

In another embodiment, the synergistic botanical based bio-insecticidal composition of the present invention exhibits synergism at specific concentration and thereby controls the plant pests/insects and protects agriculture, horticulture, olericulture and floricultural crops.

In another embodiment, the synergistic botanical based bio-insecticidal composition of the present invention is useful for controlling pests and improving yield of crop plants.

In another embodiment, the synergistic botanical based bio-insecticidal composition of the present invention exhibits biostimulant activity and thereby improves plant growth and crop yield.

In an embodiment, the present invention discloses a method for controlling the pest and promoting the growth of the plants comprising applying the present bio-insecticidal composition to the plant or to diseased affected parts of the plant in an effective amount.

The bio-insecticidal composition of the present invention has multiple advantages/benefits such as—
1. The bio-insecticidal composition of the present invention helps in controlling pests.
2. The bio-insecticidal composition of the present invention are naturally derived, easily degraded, represent the circulation of natural resources, are characterized by low toxicity and low residue, and are relatively safe for humans and other animals.
3. It is eco friendly, relatively inexpensive, bio-degradable, residue free, safe for agriculture and humans and minimizes the risk of resistance development.
4. It also helps in increasing plant growth and enhancing yield of the crops.

The following example, which includes preferred embodiments, will serve to illustrate the practice of this invention, it being understood that the particulars shown are by way of example and for purpose of illustrative discussion of preferred embodiments of the invention.

### EXAMPLES

### Example 1: Composition of Botanical Based Bio-Insecticide 1

TABLE 1

| Sr | | | |
|---|---|---|---|
| No. | Ingredient | Actual Percent | Percent Range |
| 1. | Melia dubia | 2.5% | 0.001 to 35% |
| 2. | Cinnamomum cassia | 2.5% | 0.001 to 35% |
| 3. | Piper longum | 2.5% | 0.001 to 40% |
| 4. | Lantana camara | 2.5% | 0.001 to 25% |
| 5. | Murraya koenigii | 5.0% | 0.001 to 35% |
| 6. | Acorus calamus | 5.0% | 0.001 to 35% |
| 7. | Ipomoea carnea | 5.0% | 0.001 to 25% |
| 8. | Gaur gum | 4.2% | 0.001 to 18% |
| 9. | ethoxylated castor oil | 5.8% | 0.001 to 20% |
| 10. | Carrier oil (canola oil) | 65.0% | 0.001 to 90% |

### Preparation of Botanical Based Bio-Insecticide 1:

The process for preparing Bio-Insecticide 1 comprises the steps of:
1. Melia dubia, aerial parts were extracted by using solvent extraction technique using ethyl acetate as solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using methanol and hexane as medium, followed by precipitation and crystallisation etc. to obtain purified phyto ingredients such as but not limited to alkaloids, carbohydrate, phenol, saponin, terpenoids, coumarin and glycoside etc.
2. Cinnamomum cassia, bark and leaf parts were extracted by using solvent extraction technique using ethyl acetate solvent. More precisely the entire extraction was carried between 10 degree celsius to 60 degree celsius under vacuum in accordance to preserve heat sensitive molecules. Further enrichment and purification of above obtain extract was performed by using Column chromatography by using silica gel as matrix, followed by precipitation and crystallisation etc. to obtain purified phyto ingredients such as but not limited to Cinnamic aldehyde, catechin, epicatechin, procyanidin B2, quercitrin, 3,4-dihydroxybenzaldehyde, protocatechuic acid and cinnamic acid etc.
3. Piper longum, dried fruits were extracted by using solvent extraction technique using ethyl acetate solvent. More precisely the entire extraction was carried between 10 degree celsius to 60 degree celsius under vacuum in accordance to preserve heat sensitive molecules. Further enrichment and purification of above obtained extract was performed by using Column chromatography technique by using silica gel as matrix, followed by precipitation and crystallisation etc. to obtain purified phyto ingredients such as but not limited to Piperine along with methyl piperine, iperonaline, asarinine, pellitorine, piperlongumine, piperlonguminine, Brachystamide-A, pipercide and piperidine etc.
4. Lantana camara, dried aerial parts were extracted by using solvent extraction technique using methanol solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immersible solvents as medium, followed by precipitation and crystallisation etc. to obtain purified phyto ingredients such as but not limited to tannin, saponin, flavonoids, terpenoids, alkaloids, carbohydrate, anthroquinone and polyphenols etc.
5. Murraya koenigii, dried leaf were extracted by using solvent extraction technique using ethyl acetate solvent. More precisely the entire extraction was carried between 10 degree celsius to 60 degree celsius under vacuum in accordance to preserve heat sensitive molecules. Further enrichment and purification of above obtained extract was performed by using Column chromatography technique by using silica gel as matrix, followed by precipitation and crystallisation etc. to obtain purified phyto ingredients such as but not limited to beta-caryophyllene, pyrazine, tetrakis(1-methylethyl), N, NDimethyl-Tridecylamine and d-Nerolidol etc.
6. Acorus calamus, dried roots were extracted by using solvent extraction technique using ethyl acetate solvent. More precisely the entire extraction was carried between 10 degree celsius to 60 degree celsius under vacuum in accordance to preserve heat sensitive molecules. Further enrichment and purification of above obtained extract was performed by using column chromatography technique by using silica gel as matrix, followed by precipitation and crystallisation etc. to obtain purified phyto ingredients such as but not limited to isoeugenol and their methyl ethers, camphene, P-cymene, α-selinene, bgurjunene, β-cadinene, camphor, terpinen-4-ol, aterpineol and a calacorene, acorone, acrenone, acoragermacrone, 2-deca-4,7 dienol, shyobunones, linalool and preisocalamendiol etc.
7. Ipomoea carnea, leaf parts were extracted by using solvent extraction technique using ethyl acetate solvent. More precisely the entire extraction was carried between 10 degree celsius to 60 degree celsius under vacuum in accordance to preserve heat sensitive molecules. Further enrichment and purification of above obtained extract was performed by using Column chromatography technique by using silica gel as matrix, followed by precipitation and crystallisation etc. to obtain purified phyto ingredients such as but not limited to 2-ethyl-1,3-dimethylbenzene, 2-(12-pentadecynyloxy) tetrahydro2H-pyran, 1,3-furanyl [2-hydroxy-4-methyl-2-(2-methylpropyl)cyclopentyl]-methanone, 2,2-dideuterooctadecanal, hexadecanoic acid, Linoleic acid etc.
8. Above ingredients (from step no 1 to 7) were mixed with given proportion along with organic emulsifier such as Gaur gum and bee wax and other Ionic and non-ionic emulsifier such as Poly sorbate 80 and Ethoxylated castor oil at specific concentration.
9. Blend prepared in step no 8 was mixed with carrier oils such as Sesame oil in given proportion.
10. Blend prepared in step no. 9 was stirred at 300 to 1000 rpm at 25 to 55 degrees Celsius in close mixing vessel made up of stainless steel 316 grade with outer jacket for maintaining temperature. Mixing vessel was connected with stirrer (blade stirrer, propeller stirrer, turbine stirrer, anchor stirrer, universal stirrer) for continues stirring. Towards the bottom of vessel homogenizer with the speed of 3700˜27000 rpm was attached to reduce the particle size under very high pressures, sheer, turbulence, acceleration and impact, to make them more stable and effective.
11. Blend prepare in step no 10 was passed through in line shear pump with 1800 RPM to 5800 RPM to reduce particle size in nano, so as to achieve nano emulsion with particle size ranging from 10 to 900 nano meter.
12. Further to obtain nano particles based41 formulation the blend formed in step no 11 was further passed through High-pressure homogenization (homogenization at speed of 4000 rpm to 10000 rpm and pressure max up to 4,200 bar (60,000 psi)) to reduce the particle size below 100 nano meters.
13. After passing through high pressure homogenizer the material was passed through sparkler filter having seven layers of membrane filters with pore size less than 1-2 micron to achieve proper filtration and isolation of the desired product.

The Product recovered from step 13 of the process was further quantified for the active phyto constituent by using various instruments but not limited to liquid chromatography-mass spectrometry, gas chromatography-mass spectrometry, High pressure liquid chromatograph, gas chromatography, spectroscopy compounds etc.

It was also tested for bio-efficacy against pests on different crops in vitro and in vivo, and it was compared to other chemically synthesised pesticides. The above product obtained by the process was also tested for its biostimulant activity.

### Example: 2

### Composition of Botanical Based Bio-Insecticide 2:

TABLE 2

| Sr | | | |
|---|---|---|---|
| No. | Ingredient | Actual Percent | Percent Range |
| 1. | Mentha piperita | 7.00% | 0.001 to 35% |
| 2. | Piper nigrum | 5.00% | 0.001 to 35% |
| 3. | Acorus calamus | 5.00% | 0.001 to 40% |
| 4. | Coriandrum sativum | 4.00% | 0.001 to 25% |
| 5. | Cinnamomum cassia | 4.00% | 0.001 to 35% |
| 6. | Gaur gum | 4.2% | 0.001 to 18% |
| 7. | ethoxylated castor oil | 5.8% | 0.001 to 20% |
| 9. | Carrier oil (canola oil) | 65.0% | 0.001 to 90% |

### Preparation of Botanical Based Bio-Insecticide 2:

The process for preparing the Botanical based Bio-Insecticide 2 comprises the steps of:
1. Mentha piperita aerial parts were extracted by using solvent extraction technique using ethyl acetate solvent. More precisely the entire extraction was carried between 10 degree celsius to 60 degree celsius under vacuum in accordance to preserve heat sensitive molecules. Further enrichment and purification of above obtain extract was performed by using liquid-liquid extraction technique by using immersible solvents as medium, followed by precipitation and crystallisation etc. to obtain purified phyto ingredients such as but not limited to menthol, menthofuran, menthyl acetate, menthone, and 1,8-cineole etc.
2. Piper nigrum dried fruits were extracted by using solvent extraction technique using ethyl acetate solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immersible solvents as medium, followed by precipitation and crystallisation etc. to obtain purified phyto ingredients such as but not limited to Piperine, amides, piperidine, pyrrolidines etc.
3. Acorus calamus dried roots were extracted by using solvent extraction technique by using ethyl acetate solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immersible solvents as medium, followed by precipitation and crystallisation etc. to obtain purified phyto ingredients such as but not limited to isoeugenol and their methyl ethers, camphene, P-cymene, α-selinene, bgurjunene, β-cadinene, camphor, terpinen-4-ol, aterpineol and a calacorene, acorone, acrenone, acoragermacrone, 2-deca-4,7 dienol, shyobunones, linalool and preisocalamendiol etc.
4. Coriandrum sativum dried seeds were extracted by using solvent extraction technique by using methanol solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immersible solvents as medium, followed by precipitation and crystallisation etc. to obtain purified phyto ingredients such as but not limited to Linalool, neryl acetate, γ-terpinene and α-pinene etc.
5. Cinnamomum cassia bark and leaf parts were extracted by using solvent extraction technique by using ethyl acetate as solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immersible solvents as medium, followed by precipitation and crystallisation etc. to obtain purified phyto ingredients such as but not limited to Cinnam aldehyde, catechin, epicatechin, procyanidin B2, quercitrin, 3,4-dihydroxybenzaldehyde, protocatechuic acid and cinnamic acid etc.
6. Above ingredients (from step no 1 to 5) were mixed with given proportion along with organic emulsifier such as Gaur gum and other Ionic and non-ionic emulsifier such as Poly sorbate 80 and monoglycerides at specific concentration.
7. Blend prepared in step no 6 was mixed with carrier oils such as Sesame oil in given proportion.
8. Blend prepared in step no. 7 was stirred at 300 to 1000 rpm at 25 to 55 degrees Celsius in close mixing vessel made up of stainless steel 316 grade with outer jacket for maintaining temperature. mixing vessel is connected with stirrer (blade stirrer, propeller stirrer, turbine stirrer, anchor stirrer, universal stirrer) for continues stirring. towards the bottom of vessel homogenizer with the speed of 3700˜27000 rpm is attached to reduce the particle size under very high pressures, sheer, turbulence, acceleration and impact, to make them more stable and effective.
9. Blend prepare in step no 8 was pass through in line shear pump with 1800 RPM to 5800 RPM to reduce particle size in nano, so as to achieve nano emulsion with particle size ranging from 10 to 900 nano meter.
10. Further to obtain nano particles based formulation the blend formed in step no 9 was further pass-through High-pressure homogenization (homogenization at speed of 4000 rpm to 10000 rpm and pressure max up to 4,200 bar (60,000 psi)) will reduce the particle size below 100 nano meters.
11. After passing through high pressure homogenizer the material was passed through sparkler filter having seven layers of membrane filters with pore size less than 1-2 micron to achieve proper filtration.

Product recovered from step 11 of the process was further quantified for the active phyto constituent by using various instruments but not limited to liquid chromatography-mass spectrometry, gas chromatography-mass spectrometry, High pressure liquid chromatograph, gas chromatography, spectroscopy compounds etc.

It was also tested for bio-efficacy against pests on different crops in vitro and in vivo, and it is compared to other chemically synthesised pesticides.

### Example: 3

### Composition of Botanical Based Bio-Insecticide 3:

| Sr | | | |
|---|---|---|---|
| No. | Ingredient | Actual Percent | Percent Range |
| 1. | Piper longum | 5.0% | 0.001 to 35% |
| 2. | Syzygium aromaticum | 5.0% | 0.001 to 35% |
| 3. | Mentha piperita | 5.0% | 0.001 to 40% |
| 4. | Coriandrum sativum | 5.0% | 0.001 to 25% |
| 5. | Acorus calamus | 10.0% | 0.001 to 35% |
| 6. | Gaur gum | 4.2% | 0.001 to 18% |
| 7. | ethoxylated castor oil | 5.8% | 0.001 to 20% |
| 8. | Carrier oil (canola oil) | 60.0% | 0.001 to 90% |

### Process of Preparation of Botanical Based Bio-Insecticide 3:

The process for preparing Botanical based Bio-Insecticide 3 comprises the steps:
1. Piper nigrum dried fruits were extracted by using solvent extraction technique by using ethyl acetate as solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immersible solvents as medium, followed by precipitation and crystallisation etc. to obtain purified phyto ingredients such as but not limited to Piperine, amides, piperidine, pyrrolidines etc.
2. Syzygium aromaticum dried fruits were extracted by using solvent extraction technique by using ethyl acetate as solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immersible solvents as medium, followed by precipitation and crystallisation etc. to obtain purified phyto ingredients such as but not limited to Eugenyl acetate, eugenol, and β-caryophyllene etc.
3. Mentha piperita aerial parts were extracted by using solvent extraction technique by using ethyl acetate as solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immersible solvents as medium, followed by precipitation and crystallisation etc. to obtain purified phyto ingredients such as but not limited to menthol, menthofuran, menthyl acetate, menthone, and 1,8-cineole etc.
4. Coriandrum sativum dried seeds were extracted by using solvent extraction technique by using methanol as solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immersible solvents as medium, followed by precipitation and crystallisation etc. to obtain purified phyto ingredients such as but not limited to Linalool, neryl acetate, γ-terpinene and α-pinene etc.
5. Acorus calamus dried roots were extracted by using solvent extraction technique by using ethyl acetate solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immersible solvents as medium, followed by precipitation and crystallisation etc. to obtain purified phyto ingredients such as but not limited to isoeugenol and their methyl ethers, camphene, P-cymene, α-selinene, bgurjunene, β-cadinene, camphor, terpinen-4-ol, aterpineol and a calacorene, acorone, acrenone, acoragermacrone, 2-deca-4,7 dienol, shyobunones, linalool and preisocalamendiol etc.
6. Coriandrum sativum dried seeds were extracted by using solvent extraction techniques by using methanol as solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immersible solvents as medium, followed by precipitation and crystallisation etc. to obtain purified phyto ingredients such as but not limited to Linalool, neryl acetate, γ-terpinene and α-pinene etc.
7. Above ingredients (from step no 1 to 6) were mixed with given proportion along with organic emulsifier such as Bee wax and gaur gum and other Ionic and non-ionic emulsifier such as Poly sorbate 80 and monoglycerides at specific concentration.
8. Blend prepared in step no 7 was mixed with carrier oils such as Anethum graveolens oil in given proportion.
9. Blend prepared in step no. 8 was stirred at 300 to 1000 rpm at 25 to 55 degrees Celsius in close mixing vessel made up of stainless steel 316 grade with outer jacket for maintaining temperature. Mixing vessel was connected with stirrer (blade stirrer, propeller stirrer, turbine stirrer, anchor stirrer, universal stirrer) for continues stirring towards the bottom of vessel homogenizer with the speed of 3700˜27000 rpm is attached to reduce the particle size under very high pressures, sheer, turbulence, acceleration and impact, to make them more stable and effective.
10. Blend prepare in step no 9 was passed through in line shear pump with 1800 RPM to 5800 RPM to reduce particle size in nano, so as to achieve nano emulsion with particle size ranging from 10 to 900 nano meter.
11. Further to obtain nano particles based formulation the blend formed in step no 10 was further pass-through High-pressure homogenization (homogenization at speed of 4000 rpm to 10000 rpm and pressure max up to 4,200 bar (60,000 psi)) to reduce the particle size below 100 nano meters.
12. After passing through high pressure homogenizer the material was passed through sparkler filter having seven layers of membrane filters with pore size less than 1-2 micron to achieve proper filtration.

Product recovered from step 12 of the process was further quantified for the active phyto constituent by using various instruments but not limited to liquid chromatography-mass spectrometry, gas chromatography-mass spectrometry, High pressure liquid chromatograph, gas chromatography, spectroscopy compounds etc.

It was also tested for bio-efficacy against pests on different crops in vitro and in vivo, and was compared to other chemically synthesised pesticides.

### Example: 4

### Composition of Botanical Based Bio-Insecticide 4:

| Sr | | | |
|---|---|---|---|
| No. | Ingredient | Actual Percent | Percent Range |
| 1. | Argemone mexicana | 2.50% | 0.001 to 35% |
| 2. | Datura stramonium | 2.50% | 0.001 to 35% |
| 3. | Piper longum | 2.50% | 0.001 to 40% |
| 4. | Lantana camara | 2.50% | 0.001 to 25% |
| 5. | Brassica juncea | 5.00% | 0.001 to 35% |
| 6. | Acorus calamus | 5.00% | 0.001 to 35% |
| 7. | Ipomoea carnea | 5.00% | 0.001 to 25% |
| 8. | Gaur gum | 4.2% | 0.001 to 18% |
| 9. | ethoxylated castor oil | 5.8% | 0.001 to 20% |
| 10. | Carrier oil (canola oil) | 65.0% | 0.001 to 90% |

### Preparation of Botanical Based Bio-Insecticide 4:

The process for preparing Bio-Insecticide 4 comprises the steps of:
1. Argemone mexicana, aerial parts were extracted by using solvent extraction technique by using ethyl acetate as solvent. More precisely the entire extraction was carried between 10 degree celsius to 60 degree celsius under vacuum in accordance to preserve heat sensitive molecules. Further enrichment and purification of above obtained extract was performed by using column chromatography technique by using silica gel as matrix, followed by precipitation and crystallisation etc. to obtain purified phyto ingredients such as but not limited to berberine, cryptopine, coptisine, muramine, scoulerine, stylopine, thalifoline and protopine etc.
2. Datura stramonium, bark and leaf parts were extract by using solvent extraction technique by using methanol and acetone as solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immersible solvent as medium, followed by precipitation and crystallisation etc. to obtain purified phyto ingredients such as but not limited to Atropine, scopolamine and hyoscyamine etc.
3. Piper longum, dried fruits were extract by using solvent extraction technique using ethyl acetate as solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immiscible solvents as medium, followed by precipitation and crystallisation etc. to obtain purified phyto ingredients such as but not limited to Piperine along with methyl piperine, iperonaline, asarinine, pellitorine, piperlongumine, piperlonguminine, Brachystamide-A, pipercide and piperidine etc.
4. Lantana camara, dried aerial parts were extracted by using solvent extraction technique using methanol as solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immersible solvents as medium, followed by precipitation and crystallisation etc. to obtain purified phyto ingredients such as but not limited to tannin, saponin, flavonoids, terpenoids, alkaloids, carbohydrate, anthroquinone and polyphenols etc.
5. Brassica juncea, dried leaf were extracted by using solvent extraction technique using acetone and methanol as solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immersible solvents as medium, followed by precipitation and crystallisation etc. to obtain purified phyto ingredients such as but not limited to Brassinolide, polyphenols, phenolic acids, flavonoids, and carotenoids (zeaxanthin, lutein, β-carotene) etc.
6. Acorus calamus, dried roots were extracted by using solvent extraction technique using ethyl acetate as solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immersible solvents as medium, followed by precipitation and crystallisation etc. to obtain purified phyto ingredients such as but not limited to isoeugenol and their methyl ethers, camphene, P-cymene, α-selinene, bgurjunene, β-cadinene, camphor, terpinen-4-ol, aterpineol and a calacorene, acorone, acrenone, acoragermacrone, 2-deca-4,7 dienol, shyobunones, linalool and preisocalamendiol etc.
7. Ipomoea carnea, leaf parts were extracted by using solvent extraction technique using methanol solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immersible solvents as medium, followed by precipitation and crystallisation etc. to obtain purified phyto ingredients such as but not limited to 2-ethyl-1,3-dimethylbenzene, 2-(12-pentadecynyloxy) tetrahydro2H-pyran, 3-furanyl [2-hydroxy-4-methyl-2-(2-methylpropyl)cyclopentyl]-methanone, 2,2-dideuterooctadecanal, hexadecanoic acid, Linoleic acid etc.
8. Above ingredients (from step no 1 to 7) were mixed with given proportion along with organic emulsifier such as Bee wax and gaur gum and other Ionic and non-ionic emulsifier such as Poly sorbate 80 and monoglycerides to at specific concentration.
9. Blend prepared in step no 8 was mixed with carrier oils such as Canola oil in given proportion.
10. Blend prepared in step no. 9 was stirred at 300 to 1000 rpm at 25 to 55 degrees Celsius in close mixing vessel made up of stainless steel 316 grade with outer jacket for maintaining temperature. Mixing vessel was connected with stirrer (blade stirrer, propeller stirrer, turbine stirrer, anchor stirrer, universal stirrer) for continues stirring towards the bottom of vessel homogenizer with the speed of 3700˜27000 rpm was attached to reduce the particle size under very high pressures, sheer, turbulence, acceleration and impact, to make them more stable and effective.
11. Blend prepare in step no 10 was passed through in line shear pump with 1800 RPM to 5800 RPM to reduce particle size in nano, so as to achieve nano emulsion with particle size ranging from 10 to 900 nano meter.
12. Further to obtain nano particles based formulation the blend formed in step no 11 was further pass-through High-pressure homogenization (homogenization at speed of 4000 rpm to 10000 rpm and pressure max up to 4,200 bar (60,000 psi)) to reduce the particle size below 100 nano meters.
13. After passing through high pressure homogenizer the material was passed through sparkler filter having seven layers of membrane filters with pore size less than 1-2 micron to achieve proper filtration.

Product recovered from step 13 of the process was further quantified for the active phyto constituent by using various instruments but not limited to liquid chromatography-mass spectrometry, gas chromatography-mass spectrometry, High pressure liquid chromatograph, gas chromatography, spectroscopy compounds etc.

It was also tested for bio-efficacy against pests on different crops in vitro and in vivo, and was compared to other chemically synthesised pesticides.

### Example: 5

### Composition of Botanical Based Bio-Insecticide 5:

TABLE 5

| Sr | | | |
|---|---|---|---|
| No. | Ingredient | Actual Percent | Percent Range |
| 1. | Lantana camara | 3.00% | 0.001 to 35% |
| 2. | Piper nigrum | 2.50% | 0.001 to 35% |
| 3. | Cinnamomum camphora | 5.00% | 0.001 to 40% |
| 4. | Piper longum | 2.25% | 0.001 to 25% |
| 5. | Ocimum santum | 3.00% | 0.001 to 35% |
| 6. | Thymus vulgaris | 4.50% | 0.001 to 35% |
| 7. | Citrus limon | 5.00% | 0.001 to 25% |
| 8. | Gaur gum | 4.2% | 0.001 to 18% |
| 9. | ethoxylated castor oil | 5.8% | 0.001 to 20% |
| 10. | Carrier oil (canola oil) | 64.75% | 0.001 to 90% |

### Preparation of Botanical Based Bio-Insecticide 5:

The process for preparing Bio-Insecticide 5 comprises the steps of:
1. Lantana camara aerial parts were extracted by using solvent extraction technique using methanol as solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immersible solvents as medium, followed by precipitation and crystallisation etc. to obtain purified phyto ingredients such as but not limited to tannin, saponin, flavonoids, terpenoids, alkaloids, carbohydrate, anthroquinone and polyphenols etc.
2. Piper nigrum dried fruit were extract by using solvent extraction technique using ethyl acetate as solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immersible solvents as medium, followed by precipitation and crystallisation etc. to obtain purified phyto ingredients such as but not limited to Piperine, amides, piperidine, pyrrolidines etc.
3. Cinnamomum camphora dried leaf and bark were extracted by using solvent extraction technique using ethyl acetate as solvent. More precisely the entire extraction was carried between 10 degree celsius to 60 degree celsius under vacuum in accordance to preserve heat sensitive molecules. Further enrichment and purification of above obtained extract was performed by using liquid extraction technique by using immersible solvents as medium, followed by precipitation and crystallisation etc. to obtain purified phyto ingredients such as but not limited to linalool, 1,8-cineole, α-terpineol, isoborneol, β-phellandrene, and camphor etc.
4. Piper longum dried aerial parts were extracted by using solvent extraction techniques using ethyl acetate as solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immersible solvents as medium, followed by precipitation and crystallisation etc. to obtain purified phyto ingredients such as but not limited to Piperine along with methyl piperine, iperonaline, asarinine, pellitorine, piperlongumine, piperlonguminine, Brachystamide-A, pipercide and piperidine etc.
5. Ocimum santum dried aerial parts were extracted by using solvent extraction technique using ethyl acetate as solvent. More precisely the entire extraction was carried between 10 degree celsius to 60 degree celsius under vacuum in accordance to preserve heat sensitive molecules. Further enrichment and purification of above obtained extract was performed by using Column chromatography technique by using silica gel as matrix, followed by precipitation and crystallisation etc. to obtain purified phyto ingredients such as but not limited to oleanolic acid, rosmarinic acid, ursolic acid, eugenol, methyl eugenol, linalool, carvacrol, β elemene, β caryophyllene, germacrene etc.
6. Thymus vulgaris dried roots were extracted by using solvent extraction technique using methanol as solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immersible solvents as medium, followed by precipitation and crystallisation etc. to obtain purified phyto ingredients such as but not limited to 1,8-cineole, camphor, α-pinene, camphene, carvarcrol and thymol etc.
7. Citrus limon leaf & dried fruit parts were extracted by using steam distillation by using steam for extraction of volatile compounds by using steam as solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immersible solvents as medium, followed by precipitation and crystallisation etc. to obtain purified phyto ingredients such as but not limited to Pinene (β), Limonene, Terpinene (γ), Terpinen-4-ol, Neral Geranial etc.
8. Above ingredients (from step no 1 to 7) were mixed with given proportion along with organic emulsifier such as rice bran wax and gaur gum and other Ionic and non-ionic emulsifier such as Poly sorbate 80 and Ethoxylated castor oil at specific concentration.
9. Blend prepared in step no 8 was mixed with carrier oils such as canola oil in given proportion.
10. Blend prepared in step no. 9 was stirred at 300 to 1000 rpm at 25 to 55 degrees Celsius in close mixing vessel made up of stainless steel 316 grade with outer jacket for maintaining temperature mixing vessel is connected with stirrer (blade stirrer, propeller stirrer, turbine stirrer, anchor stirrer, universal stirrer) for continues stirring towards the bottom of vessel homogenizer with the speed of 3700˜27000 rpm is attached to reduce the particle size under very high pressures, sheer, turbulence, acceleration and impact, to make them more stable and effective.
11. Blend prepare in step no 10 was passed through in line shear pump with 1800 RPM to 5800 RPM to reduce particle size in nano, so as to achieve nano emulsion with particle size ranging from 10 to 900 nano meter.
12. Further to obtain nano particles based formulation the blend formed in step no 11 was further passed through High-pressure homogenization (homogenization at speed of 4000 rpm to 10000 rpm and pressure max up to 4,200 bar (60,000 psi)) will reduce the particle size below 100 nano meters.
13. After passing through high pressure homogenizer the material was passed through sparkler filter having seven layers of membrane filters with pore size less than 1-2 micron to achieve proper filtration.

Product recovered from step 13 of the process was further quantified for the active phyto constituent by using various instruments but not limited to liquid chromatography-mass spectrometry, gas chromatography-mass spectrometry, High pressure liquid chromatograph, gas chromatography, spectroscopy compounds etc.

It was also tested for bio-efficacy against pests on different crops in vitro and in vivo, and was compared to other chemically synthesised pesticides.

### Example: 6

### Composition of Botanical Based Bio-Insecticide 6:

TABLE 6

| Sr | | | |
|---|---|---|---|
| No. | Ingredient | Actual Percent | Percent Range |
| 1. | Abrus precatorius | 3.0% | 0.001 to 35% |
| 2. | Acorus calamus | 5.0% | 0.001 to 35% |
| 3. | Jatropha integerrima | 5.0% | 0.001 to 40% |
| 4. | Piper longum | 2.0% | 0.001 to 25% |
| 5. | Acacia concinna | 4.0% | 0.001 to 35% |
| 6. | Cuminum cyminum | 3.0% | 0.001 to 35% |
| 8. | Gaur gum | 4.2% | 0.001 to 18% |
| 9. | ethoxylated castor oil | 5.8% | 0.001 to 20% |
| 10. | Carrier oil (canola oil) | 68.0% | 0.001 to 90% |

### Preparation of Botanical Based Bio-Insecticide 6:

The process for preparing Bio-Insecticide 6 comprises the steps of:
1. Abrus precatorius dried seeds were extracted by using solvent extraction technique using acetone as solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immersible solvents as medium, followed by precipitation and crystallisation etc. to obtain purified phyto ingredients such as but not limited to Abrin, β-carotene, glutathione, α-tocopherol etc.
2. Acorus calamus dried roots were extracted by using solvent extraction technique using ethyl acetate as solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immersible solvents as medium, followed by precipitation and crystallisation etc. to obtain purified phyto ingredients such as but not limited to isoeugenol and their methyl ethers, camphene, P-cymene, α-selinene, bgurjunene, β-cadinene, camphor, terpinen-4-ol, aterpineol and a calacorene, acorone, acrenone, acoragermacrone, 2-deca-4,7 dienol, shyobunones, linalool and preisocalamendiol etc.
3. Jatropha integerrima dried seeds and bark were extracted by using solvent extraction technique using methanol as solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immersible solvents as medium, followed by precipitation and crystallisation etc. to obtain purified phyto ingredients such as but not limited to Anthocyanin, carbohydrate, coumarin, glycoside, phenol, protein, saponin, and tannin etc.
4. Piper longum dried aerial parts were extracted by using solvent extraction technique using ethyl acetate as solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immersible solvents as medium, followed by precipitation and crystallisation etc. to obtain purified phyto ingredients such as but not limited to Piperine along with methyl piperine, iperonaline, asarinine, pellitorine, piperlongumine, piperlonguminine, Brachystamide-A, pipercide and piperidine etc.
5. Acacia concinna dried aerial parts were extracted by using Hydrophilic extraction technique using water as solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immersible solvents as medium, followed by precipitation and crystallisation etc. to obtain purified phyto ingredients such as but not limited to lupeol, spinasterol, acacic acid, lactone and natural sugars glucose, arabinose and rhamnose etc.
6. Cuminum cyminum dried roots were extracted by using extraction technique using ethyl acetate as solvent. More precisely the entire extraction was carried between 10 degree celsius to 60 degree celsius under vacuum in accordance to preserve heat sensitive molecules. Further enrichment and purification of above obtained extract was performed by using liquid-liquid extraction technique by using immersible solvents as medium, followed by precipitation and crystallisation etc. to obtain purified phyto ingredients such as but not limited to Cumin aldehyde, alkaloid, anthraquinone, coumarin, flavonoid, glycoside, protein, resin, saponin, tannin and steroid etc.
7. Above ingredients (from step no 1 to 6) were mixed with given proportion along with organic emulsifier such as Gaur gum and bee wax and other Ionic and non-ionic emulsifier such as polysorbate 80 at specific concentration.
8. Blend prepared in step no 7 was mixed with carrier oils such as Sesame oil in given proportion.
9. Blend prepared in step no. 8 was stirred at 300 to 1000 rpm at 25 to 55 degrees Celsius in close mixing vessel made up of stainless steel 316 grade with outer jacket for maintaining temperature. Mixing vessel was connected with stirrer (blade stirrer, propeller stirrer, turbine stirrer, anchor stirrer, universal stirrer) for continues stirring towards the bottom of vessel homogenizer with the speed of 3700˜27000 rpm is attached to reduce the particle size under very high pressures, sheer, turbulence, acceleration and impact, to make them more stable and effective.
10. Blend prepare in step no 9 was passed through in line shear pump with 1800 RPM to 5800 RPM to reduce particle size in nano, so as to achieve nano emulsion with particle size ranging from 10 to 900 nano meter.
11. Further to obtain nano particles based formulation the blend formed in step no 10 was further passed through High-pressure homogenization (homogenization at speed of 4000 rpm to 10000 rpm and pressure max up to 4,200 bar (60,000 psi)) to reduce the particle size below 100 nano meters.
12. After passing through high pressure homogenizer the material was passed through sparkler filter having seven layers of membrane filters with pore size less than 1-2 micron to achieve proper filtration.

Product recovered from step 12 of the process was further quantified for the active phyto constituent by using various instruments but not limited to liquid chromatography-mass spectrometry, gas chromatography-mass spectrometry, High pressure liquid chromatograph, gas chromatography, spectroscopy compounds etc.

It was also tested for bio-efficacy against pests on different crops in vitro and in vivo, and it is compared to other chemically synthesised pesticides.

### Example 7: Plant Detail Along with Mode of Actions

TABLE 7

| | | Different form | | | | |
|---|---|---|---|---|---|---|
| | | of botanical | Part | | | |
| | Class of | based Bio- | Percentage | | | |
| Sr. | Bio- | pesticides used | used in | Percent | Pesticidal | Mode of |
| No. | pesticides | in formulation | formulation | Range | Properties | action |
| 1. | Thymus | Thymus | 6.0% | 0.001- | Fungicidal | Disrupts cell |
| | vulgaris | vulgaris extract | | 35% | activity | wall & |
| | | Enriched & | 3.5% | 0.001- | Bactericidal | membrane |
| | | Purified extract | | 25% | Activity | integrity |
| | | of Thymus | | | Insecticidal | Affecting |
| | | vulgaris | | | activity | Water |
| | | (Purified Phyto | 1.0% | 0.001- | Nematicidal | Balance |
| | | ingredients or | | 15% | activity | Oviposition |
| | | marker | | | virucidal | deterrence |
| | | compounds) | | | activity | Disturb |
| | | 1,8-cineole, | | | | central |
| | | camphor, | | | | nervous |
| | | α-pinene, | | | | system |
| | | camphene, | | | | |
| | | carvarcrol and | | | | |
| | | thymol | | | | |
| 2. | Cinnamomum | Cinnamomum | 7.0% | 0.001- | Fungicidal | Disrupts cell |
| | cassia | cassia extract | | 35% | activity | wall & |
| | | Enriched & | 3.0% | 0.001- | Bactericidal | membrane |
| | | Purified extract | | 25% | Activity | integrity |
| | | of | | | Insecticidal | Anti feeding |
| | | Cinnamomum | | | activity | Oviposition deterrence |
| | | cassia | | | Nematicidal | Disturb |
| | | (Purified Phyto | 1.2% | 0.001- | activity | central |
| | | ingredients or | | 15% | virucidal | nervous |
| | | marker | | | activity | system |
| | | compounds) | | | | Affect egg |
| | | Cinnamic | | | | hatchability |
| | | aldehyde, | | | | |
| | | catechin, | | | | |
| | | epicatechin, | | | | |
| | | quercitrin, | | | | |
| | | protocatechuic | | | | |
| | | acid and | | | | |
| | | cinnamic acid | | | | |
| 3. | Cuminum | Cuminum | 8.0% | 0.001- | Fungicidal | Interfere with |
| | cyminum | cyminum | | 45% | activity | protein |
| | | extract | | | Bactericidal | synthesis |
| | | Enriched & | 4.8% | 0.001- | Activity | Disrupts cell |
| | | Purified extract | | 30% | Insecticidal | wall & |
| | | of Cuminum | | | activity | membrane |
| | | cyminum | | | Nematicidal | Anti feeding |
| | | (Purified Phyto | 2.0% | 0.001- | activity | Oviposition |
| | | ingredients or | | 20% | | deterrence |
| | | marker | | | | Disturb |
| | | compounds) | | | | central |
| | | Cumin | | | | nervous |
| | | aldehyde, | | | | system |
| | | alkaloid, | | | | |
| | | anthraquinone, | | | | |
| | | saponin, tannin | | | | |
| | | and steroid | | | | |
| 4. | Abrus | Abrus | 3.0% | 0.001- | Fungicidal | Inhibiting of |
| | precatorius | precatorius | | 45% | activity | proteins |
| | | extract | | | Bactericidal | synthesis |
| | | Enriched & | 1.5% | 0.001- | Activity | Anti feeding |
| | | Purified extract | | 30% | Insecticidal | Oviposition |
| | | of Abrus | | | activity | deterrence |
| | | precatorius | | | Nematicidal | Disturb |
| | | (Purified Phyto | 0.5% | 0.001- | activity | central |
| | | ingredients or | | 15% | | nervous |
| | | marker | | | | system |
| | | compounds) | | | | |
| | | Abrin and α- | | | | |
| | | tocopherol | | | | |
| 5. | Piper | Piper nigrum | 8.0% | 0.001- | Fungicidal | Inhibiting of |
| | nigrum | extract | | 35% | activity | proteins |
| | | | | | Bactericidal | synthesis |
| | | Enriched & | 2.5% | 0.001- | Activity | Anti feeding |
| | | Purified extract | | 25% | Insecticidal | Disrupts cell |
| | | of Piper | | | activity | wall & |
| | | nigrum | | | Nematicidal | membrane |
| | | (Purified Phyto | 1.5% | 0.001- | activity | |
| | | ingredients or | | 15% | virucidal | |
| | | marker | | | activity | |
| | | compounds) | | | | |
| | | Piperine, | | | | |
| | | amides, | | | | |
| | | piperidine and | | | | |
| | | pyrrolidines | | | | |
| 6. | Piper | Piper longum | 5.0% | 0.001- | Fungicidal | Inhibiting of |
| | longum | extract | | 45% | activity | proteins |
| | | Enriched & | 2.5% | 0.001- | Bactericidal | synthesis |
| | | Purified extract | | 30% | Activity | Anti feeding |
| | | of Piper | | | Insecticidal | Disrupts cell |
| | | longum | | | activity | wall & |
| | | (Purified Phyto | 1.5% | 0.001- | Nematicidal | membrane |
| | | ingredients or | | 15% | activity | |
| | | marker | | | virucidal | |
| | | compounds) | | | activity | |
| | | methyl | | | | |
| | | piperine, | | | | |
| | | Piperine | | | | |
| | | piperlongumine, | | | | |
| | | piperlonguminine, | | | | |
| | | pipercide | | | | |
| | | and piperidine | | | | |
| 7. | Syzygium | Syzygium | 5.5% | 0.001- | Fungicidal | Disrupts cell |
| | aromaticum | aromaticum | | 45% | activity | wall & |
| | | extract | | | | membrane |
| | | Enriched & | 3.5% | 0.001- | Bactericidal | integrity |
| | | Purified extract | | 30% | Activity | Anti feeding |
| | | of Syzygium | | | Insecticidal | Oviposition |
| | | aromaticum | | | activity | deterrence |
| | | (Purified Phyto | 1.5% | 0.001- | Nematicidal | Disturb |
| | | ingredients or | | 15% | activity | central |
| | | marker | | | virucidal | nervous |
| | | compounds) | | | activity | system |
| | | Eugenyl | | | | Affect egg |
| | | acetate, eugenol, | | | | hatchability |
| | | and β- | | | | |
| | | caryophyllene | | | | |
| 8. | Pongamia | Pongamia | 12.5% | 0.001- | Insecticidal | Anti feeding |
| | pinnata | pinnata extract | | 45% | activity | Oviposition |
| | | Enriched & | 5.5% | 0.001- | Nematicidal | deterrence |
| | | Purified extract | | 30% | activity | Disturb |
| | | of Pongamia | | | | central |
| | | pinnata | | | | nervous |
| | | (Purified Phyto | 2.5% | 0.001- | | system |
| | | ingredients or | | 15% | | Affect egg |
| | | marker | | | | hatchability |
| | | compounds) | | | | |
| | | karanjin, | | | | |
| | | glabrin, and | | | | |
| | | kanjone | | | | |
| 9. | Anethum | Anethum | 5.0% | 0.001- | Insecticidal | Disrupts cell |
| | graveolens | graveolens | | 45% | activity | wall & |
| | | extract | | | | membrane |
| | | Enriched & | 3.5% | 0.001- | Nematicidal | integrity |
| | | Purified extract | | 30% | activity | Anti feeding |
| | | of Anethum | | | | Oviposition |
| | | graveolens | | | | deterrence |
| | | (Purified Phyto | 2.5% | 0.001- | Fungicidal | Disturb |
| | | ingredients or | | 15% | activity | central |
| | | marker | | | | nervous |
| | | compounds) | | | | system |
| | | alkaloids, | | | | |
| | | flavonoids, | | | | |
| | | tannins and | | | | |
| | | saponins | | | | |
| 10. | Cinnamomum | Cinnamomum | 5.0% | 0.001- | Fungicidal | Disrupts cell |
| | camphora | camphora | | 45% | activity | wall & |
| | | extract | | | | membrane |
| | | Enriched & | 2.0% | 0.001- | Bactericidal | integrity |
| | | Purified extract | | 30% | Activity | Anti feeding |
| | | of | | | Insecticidal | Oviposition |
| | | Cinnamomum | | | activity | deterrence |
| | | camphora | | | | Disturb |
| | | (Purified Phyto | 1.0% | 0.001- | Nematicidal | central |
| | | ingredients or | | 15% | activity | nervous |
| | | marker | | | virucidal | system |
| | | compounds) | | | activity | |
| | | linalool, 1,8- | | | | |
| | | cineole, α- | | | | |
| | | terpineol, | | | | |
| | | and camphor | | | | |
| 11. | Acorus | Acorus | 10.0% | 0.001- | Fungicidal | Disrupts cell |
| | calamus | calamus extract | | 45% | activity | wall & |
| | | Enriched & | | | Bactericidal | membrane |
| | | Purified extract | | | Activity | integrity |
| | | of Acorus | 7.5% | 0.001- | Insecticidal | Anti feeding |
| | | calamus | | 30% | activity | Oviposition |
| | | (Purified Phyto | | | Nematicidal | deterrence |
| | | ingredients or | | | activity | Disturb |
| | | marker | 4.5% | 0.001- | virucidal | central |
| | | compounds) | | 15% | activity | nervous |
| | | methyl ethers, | | | | system |
| | | camphene, | | | | |
| | | P-cymene, | | | | |
| | | α-selinene, | | | | |
| | | bgurjunene, | | | | |
| | | β-cadinene, | | | | |
| | | camphor, | | | | |
| | | shyobunones, | | | | |
| | | linalool and | | | | |
| | | preisocalamendiol | | | | |
| 12. | Citrus | Citrus limon | 10.0% | 0.001- | Fungicidal | Disrupts cell |
| | limon | extract | | 45% | activity | wall & |
| | | Enriched & | 7.5% | 0.001- | Bactericidal | membrane |
| | | Purified extract | | 30% | Activity | integrity |
| | | of Citrus limon | | | | Anti feeding |
| | | (Purified Phyto | 4.5% | 0.001- | Insecticidal | Oviposition |
| | | ingredients or | | 15% | activity | deterrence |
| | | marker | | | Nematicidal | Disturb |
| | | compounds) | | | activity | central |
| | | Pinene (β), | | | virucidal | nervous |
| | | Limonene, | | | activity | system |
| | | Terpinene (γ), | | | | |
| | | Terpinen-4-ol, | | | | |
| | | Neral Geranial | | | | |
| 13. | Azadirachta | Azadirachta | 5.0% | 0.001- | Insecticidal | Anti feeding |
| | indica | indica extract | | 45% | activity | Oviposition |
| | | Enriched & | 3.7% | 0.001- | Nematicidal | deterrence |
| | | Purified extract | | 30% | activity | Disturb |
| | | of Azadirachta | | | Fungicidal | central |
| | | indica | | | activity | nervous |
| | | (Purified Phyto | 1.5% | 0.001- | | system |
| | | ingredients or | | 15% | | Affect egg |
| | | marker | | | | hatchability |
| | | compounds) | | | | |
| | | Annonacin, | | | | |
| | | Squamocin, | | | | |
| | | Annonin | | | | |
| | | (Purified Phyto | 2.5% | 0.001- | | |
| | | ingredients or | | 15% | | |
| | | marker | | | | |
| | | compounds) | | | | |
| | | kaempferol-3- | | | | |
| | | O and lupeol, | | | | |
| | | β - and α - | | | | |
| | | amyrin, | | | | |
| | | quercetin, | | | | |
| | | camphor, ricin, | | | | |
| | | epicatechin, | | | | |
| | | catechin, | | | | |
| | | linoleic acid | | | | |
| | | and ricinoleic | | | | |
| | | acid | | | | |
| 14. | Sesamum | Sesamum indica | 35.0% | 0.001- | Insecticidal | Anti feeding |
| | indica | extract | | 55% | activity | Oviposition |
| | | Enriched & | 10.0% | 0.001- | Nematicidal | deterrence |
| | | Purified extract | | 30% | activity | Disturb |
| | | of | | | | central |
| | | Sesamum indica | | | | nervous |
| | | (Purified Phyto | 5.6% | 0.001- | Fungicidal | system |
| | | ingredients or | | 15% | activity | Affect egg |
| | | marker | | | | hatchability |
| | | compounds) | | | | |
| | | Sesamin, | | | | |
| | | kaempferol-3- | | | | |
| | | O and lupeol, | | | | |
| | | β - and α - | | | | |
| | | amyrin, | | | | |
| | | quercetin, | | | | |
| | | camphor, ricin, | | | | |
| | | epicatechin, | | | | |
| | | catechin, | | | | |
| | | linoleic acid | | | | |
| | | and ricinoleic | | | | |
| | | acid | | | | |
| 15. | Argemone | Argemone | 10.0% | 0.001- | Fungicidal | Disrupts cell |
| | mexicana | mexicana | | 55% | activity | wall & |
| | | extract | | | | membrane |
| | | Enriched & | 8.0% | 0.001- | Bactericidal | integrity |
| | | Purified extract | | 30% | Activity | Anti feeding |
| | | of Argemone | | | Insecticidal | Oviposition |
| | | mexicana | | | activity | deterrence |
| | | (Purified Phyto | 3.0% | 0.001- | Nematicidal | Disturb |
| | | ingredients or | | 15% | activity | central |
| | | marker | | | virucidal | nervous |
| | | compounds) | | | activity | system |
| | | berberine, | | | | |
| | | cryptopine, | | | | |
| | | scoulerine, | | | | |
| | | stylopine, | | | | |
| | | sanguinarine, | | | | |
| | | thalifoline and | | | | |
| | | protopine | | | | |
| 16. | Moringa | Moringa | 12.0% | 0.001- | Insecticidal | Anti feeding |
| | oleifera | oleifera extract | | 55% | activity | Oviposition |
| | | Enriched & | 7.0% | 0.001- | Nematicidal | deterrence |
| | | Purified extract | | 30% | activity | Disturb |
| | | of Moringa | | | Plant growth | central |
| | | oleifera | | | | nervous |
| | | (Purified Phyto | 4.0% | 0.001- | Promotion | system |
| | | ingredients or | | 15% | activity | Plant growth |
| | | marker | | | | stimulation |
| | | compounds) | | | | |
| | | alkaloids, | | | | |
| | | saponins, | | | | |
| | | tannins, | | | | |
| | | steroids, | | | | |
| | | phenolic acids, | | | | |
| | | glucosinolates, | | | | |
| | | flavonoids, and | | | | |
| | | terpenes | | | | |
| 17. | Brassica | Brassica | 8.0% | 0.001- | Insecticidal | Anti feeding |
| | juncea | juncea extract | | 55% | activity | Oviposition |
| | | Enriched & | 4.0% | 0.001- | Nematicidal | deterrence |
| | | Purified extract | | 30% | activity | Disturb |
| | | of Brassica | | | Plant growth | central |
| | | juncea | | | Promotion | nervous |
| | | (Purified Phyto | 2.2% | 0.001- | activity | system |
| | | ingredients or | | 15% | | Plant growth |
| | | marker | | | | stimulation |
| | | compounds) | | | | |
| | | brassinolide, | | | | |
| | | polyphenols, | | | | |
| | | phenolic acids, | | | | |
| | | flavonoids, | | | | |
| | | carotenoids | | | | |
| | | (zeaxanthin, | | | | |
| | | lutein, β- | | | | |
| | | carotene), | | | | |
| | | alkaloids, | | | | |
| | | tannins and | | | | |
| | | saponins | | | | |
| 18. | Lantana | Lantana | 5.0% | 0.001- | Insecticidal | Anti feeding |
| | camara | camara extract | | 55% | activity | Oviposition |
| | | Enriched & | 3.0% | 0.001- | Nematicidal activity | deterrence |
| | | Purified extract | | 30% | Fungicidal | Disturb central |
| | | of Lantana | | | activity | nervous |
| | | camara | | | | system |
| | | (Purified Phyto | 1.3% | 0.001- | | Affect egg |
| | | ingredients or | | 15% | | hatchability |
| | | marker | | | | |
| | | compounds) | | | | |
| | | tannin, saponin, | | | | |
| | | flavonoids, | | | | |
| | | terpenoids, | | | | |
| | | alkaloids, | | | | |
| | | carbohydrate, | | | | |
| | | anthroquinone | | | | |
| | | and | | | | |
| | | polyphenols | | | | |
| 19. | Datura | Datura | 8.0% | 0.001- | Insecticidal | Anti feeding |
| | stramonium | stramonium | | 55% | activity | Oviposition |
| | | extract | | | Nematicidal | deterrence |
| | | Enriched & | 3.5% | 0.001- | activity | Disturb |
| | | Purified extract | | 30% | Fungicidal | central |
| | | of Datura | | | activity | nervous |
| | | stramonium | | | | system |
| | | (Purified Phyto | 2.0% | 0.001- | | Affect egg |
| | | ingredients or | | 15% | | hatchability |
| | | marker | | | | |
| | | compounds) | | | | |
| | | Atropine, | | | | |
| | | scopolamine | | | | |
| | | and | | | | |
| | | hyoscyamine | | | | |
| 20. | Melia | Melia dubia | 2.5% | 0.001- | Insecticidal | Anti feeding |
| | dubia | extract | | 55% | activity | Oviposition |
| | | Enriched & | 1.8% | 0.001- | Nematicidal | deterrence |
| | | Purified extract | | 30% | activity | Disturb |
| | | of Melia dubia | | | Fungicidal | central |
| | | (Purified Phyto | 1.0% | 0.001- | activity | nervous |
| | | ingredients or | | 15% | | system |
| | | marker | | | | Affect egg |
| | | compounds) | | | | hatchability |
| | | Alkaloids, | | | | |
| | | Carbohydrate, | | | | |
| | | Phenol, | | | | |
| | | Saponin, | | | | |
| | | Terpenoids, | | | | |
| | | Coumarin and | | | | |
| | | Glycoside | | | | |
| 21. | Murraya | Murraya | 5.0% | 0.001- | Fungicidal | Disrupts cell |
| | koenigii | koenigii extract | | 55% | activity | wall & |
| | | Enriched & | 3.5% | 0.001- | Bactericidal | membrane |
| | | Purified extract | | 30% | Activity | integrity |
| | | of Murraya | | | Insecticidal | Interfere with |
| | | koenigii | | | activity | protein |
| | | (Purified Phyto | 1.5% | 0.001- | Nematicidal | synthesis |
| | | ingredients or | | 15% | activity | Anti feeding |
| | | marker | | | virucidal | Oviposition |
| | | compounds) | | | activity | deterrence |
| | | Alkaloids, | | | | Disturb |
| | | Carbohydrate, | | | | central |
| | | Phenol, | | | | nervous |
| | | Saponin, | | | | system |
| | | Terpenoids, | | | | |
| | | Coumarin and | | | | |
| | | Glycoside | | | | |
| 22. | Ipomoea | Ipomoea | 5.0% | 0.001- | Insecticidal | Anti feeding |
| | carnea | carnea extract | | 55% | activity | Oviposition |
| | | Enriched & | 2.5% | 0.001- | Nematicidal | deterrence |
| | | Purified extract | | 30% | activity | Disturb |
| | | of Ipomoea | | | Fungicidal | central |
| | | carnea | | | activity | nervous |
| | | (Purified Phyto | 1.0% | 0.001- | virucidal | system |
| | | ingredients or | | 15% | activity | Affect egg |
| | | marker | | | | hatchability |
| | | compounds) | | | | |
| | | 2-ethyl-1,3- | | | | |
| | | dimethylbenze | | | | |
| | | ne, 2-(12- | | | | |
| | | pentadecynyloxy) | | | | |
| | | tetrahydro2H- | | | | |
| | | pyran, 3- | | | | |
| | | furanyl[2- | | | | |
| | | hydroxy-4- | | | | |
| | | methyl-2-- (2- | | | | |
| | | methylpropyl)c | | | | |
| | | yclopentyl]- | | | | |
| | | methanone, 2, | | | | |
| | | 2- | | | | |
| | | dideuterooctad | | | | |
| | | ecanal and | | | | |
| | | hexadecanoic | | | | |
| | | acid | | | | |
| 23. | Ocimum | Ocimum | 6.0% | 0.001- | Fungicidal | Disrupts cell |
| | sanctum | sanctum extract | | 55% | activity | wall & |
| | | Enriched & | 4.5% | 0.001- | Bactericidal | membrane |
| | | Purified extract | | 30% | Activity | integrity |
| | | of Ocimum | | | Insecticidal | Interfere with |
| | | sanctum | | | activity | protein |
| | | (Purified Phyto | 2.5% | 0.001- | Nematicidal | synthesis |
| | | ingredients or | | 15% | activity | Anti feeding |
| | | marker | | | virucidal | Oviposition |
| | | compounds) | | | activity | deterrence |
| | | oleanolic acid, | | | | Disturb |
| | | rosmarinic | | | | central |
| | | acid, ursolic | | | | nervous |
| | | acid, eugenol, | | | | system |
| | | methyl | | | | |
| | | eugenol, | | | | |
| | | linalool, | | | | |
| | | carvacrol, β | | | | |
| | | elemene and β | | | | |
| | | caryophyllene | | | | |
| 24. | Jatropha | Jatropha | 5.0% | 0.001- | Insecticidal | Anti feeding |
| | integerrima | integerrima | | 55% | activity | Oviposition |
| | | extract | | | Nematicidal | deterrence |
| | | Enriched & | 3.0% | 0.001- | activity | Disturb |
| | | Purified extract | | 30% | Fungicidal | central |
| | | of Jatropha | | | activity | nervous |
| | | integerrima | | | virucidal | system |
| | | Purified Phyto | 2.0% | 0.001- | activity | Affect egg |
| | | ingredients or | | 15% | | hatchability |
| | | marker | | | | |
| | | compounds) | | | | |
| | | Anthocyanin, | | | | |
| | | coumarin, | | | | |
| | | glycoside, | | | | |
| | | phenol, | | | | |
| | | saponin, and | | | | |
| | | tannin | | | | |
| 25. | Acacia | Acacia | 13.0% | 0.001- | Insecticidal | Anti feeding |
| | concinna | concinna | | 55% | activity | Oviposition |
| | | extract | | | Fungicidal | deterrence |
| | | Enriched & | 7.0% | 0.001- | activity | Disturb |
| | | Purified extract | | 30% | | central |
| | | of Acacia | | | | nervous |
| | | concinna | | | | system |
| | | Purified Phyto | 5.0% | 0.001- | | Disrupts cell |
| | | ingredients or | | 15% | | wall & |
| | | marker | | | | membrane |
| | | compounds) | | | | integrity |
| | | lupeol, | | | | |
| | | spinasterol, | | | | |
| | | acacic acid, | | | | |
| | | lactone and | | | | |
| | | natural sugars | | | | |
| | | glucose. | | | | |
| 26. | Mentha | Mentha | 7.0% | 0.001- | Fungicidal | Disrupts cell |
| | piperita | piperita extract | | 55% | activity | wall & |
| | | Enriched & | 4.2% | 0.001- | Bactericidal | membrane |
| | | Purified extract | | 30% | Activity | integrity |
| | | of Mentha | | | Insecticidal | Interfere with |
| | | piperita | | | activity | protein |
| | | Purified Phyto | 2.8% | 0.001- | Nematicidal | synthesis |
| | | ingredients or | | 15% | activity | Anti feeding |
| | | marker | | | virucidal | Oviposition |
| | | compounds) | | | activity | deterrence |
| | | lupeol, | | | | Disturb |
| | | spinasterol, | | | | central |
| | | acacic acid, | | | | nervous |
| | | lactone and | | | | system |
| | | natural sugars | | | | |
| | | glucose. | | | | |
| 27. | Coriandrum | Coriandrum | 5.0% | 0.001- | Insecticidal | Anti feeding |
| | sativum | sativum extract | | 55% | activity | Oviposition |
| | | Enriched & | 2.2% | 0.001- | Nematicidal | deterrence |
| | | Purified extract | | 30% | | |
| | | of Coriandrum | | | | |
| | | sativum | | | | |
| | | Purified Phyto | 1.8% | 0.001- | activity | Disturb |
| | | ingredients or | | 15% | Fungicidal activity | central |
| | | marker | | | virucidal | nervous |
| | | compounds) | | | activity | system |
| | | Linalool, neryl | | | | Affect egg |
| | | acetate, γ- | | | | hatchability |
| | | terpinene and | | | | |
| | | α-pinene | | | | |
| 28. | Annona | Annona | 2.5% | 0.001- | Insecticidal | Anti feeding |
| | squamosa | squamosa | | 45% | activity | Oviposition |
| | | extract | | | Nematicidal | deterrence |
| | | Enriched & | 1.8% | 0.001- | activity | Disturb |
| | | Purified extract | | 30% | Fungicidal | central |
| | | of Annona | | | activity | nervous |
| | | squamosa | | | | system |
| | | (Purified Phyto | 1.0% | 0.001- | | |
| | | ingredients or | | 15% | | |
| | | marker | | | | |
| | | Affect egg | | | | |
| | | compounds) | | | | |
| | | hatchability | | | | |
| | | Annonacin, | | | | |
| | | Squamocin, | | | | |
| | | Annonin | | | | |

### Example 8: Effect of Bio-Stimulant on Growth and Yield of Cauliflower

Name of the Product: Bio Insecticide
Variety: Kimaya (Syngenta) Crop: Cauliflower

### Material and Method

A field experiment was conducted at Kay Bee R&D Farm, Phaltan (MS) during Kharif season 2022-2023. The experimental site under subtropical climatic condition. The experiment consisted of ten treatments as presented in Table 8. The cauliflower variety Kimaya (Syngenta) was used and experiment was arranged in Randomized Block Design with three replications. The net size of each plot was 4.80×3.60 m². All the recommended practices were applied as per stage and condition of crop. The spraying of the formulation was conducted at 15 and 30 Days After Transplanting (DAT). The morphological data like plant height and plant spread were recorded at physiological maturity. The yield and yield contributing data were recorded as per the crop stage of occurrence. Collected data on different parameters were statistically analyzed by using “Analysis of Variance Method (ANOVA)” (Panse and Sukhatme, 1967).

### Results

The data on effect of bio-stimulant on growth and yield of cauliflower were recorded statistically significant result and are furnished in Table 8.

TABLE 8
Response of Bio-stimulants on Cauliflower.

| | | | Plant | Plant | Curd | Curd | Curd |
|---|---|---|---|---|---|---|---|
| Tr. | | Dose | height | Spread | Maturity | Weight | Yield |
| No. | Treatment. Code | (ml/lit.) | (cm) | (cm) | Days | (Kg) | (t/Ha) |
| 1 | Bio Insecticide | 0.50 | 47.32 | 60.12 | 63.57 | 0.75 | 29.64 |
| 2 | Bio Insecticide | 1.00 | 46.37 | 62.38 | 62.77 | 0.83 | 31.65 |
| 3 | Bio Insecticide | 2.00 | 48.54 | 65.37 | 62.37 | 0.98 | 32.71 |
| 4 | Nutrozen (Plant | 2.00 | 52.45 | 69.00 | 59.37 | 1.20 | 35.92 |
| | derived nutrition | | | | | | |
| | along with the | | | | | | |
| | necessary | | | | | | |
| | hormones, vitamins | | | | | | |
| | and sea weed | | | | | | |
| | extract) | | | | | | |
| 5 | Isabion (Organic | 2.00 | 52.39 | 69.31 | 60.47 | 1.15 | 34.65 |
| | fraction derived | | | | | | |
| | from Amino Acid | | | | | | |
| | and peptides | | | | | | |
| | mixture 62.5%) | | | | | | |
| 6 | Biozyme Crop+ | 2.00 | 52.19 | 68.26 | 61.47 | 1.00 | 33.56 |
| | (Seaweed Extract, | | | | | | |
| | Proteins, | | | | | | |
| | Carbohydrates, | | | | | | |
| | Inorganic Salts and | | | | | | |
| | Other Inherent | | | | | | |
| | Nutrients) 22% | | | | | | |
| 7 | Double | 0.50 | 47.69 | 61.54 | 63.27 | 0.79 | 30.23 |
| | (Homobrassinolide | | | | | | |
| | 0.04%) | | | | | | |
| 8 | Macarena | 1.25 | 51.45 | 67.34 | 59.83 | 1.02 | 34.02 |
| | (Fermented extract | | | | | | |
| | Nutrient 15%) | | | | | | |
| 9 | Hoshi (Gibberellic | 2.00 | 49.78 | 65.93 | 61.97 | 0.93 | 33.23 |
| | Acid 0.001%) | | | | | | |
| 10 | Water Control | — | 45.62 | 59.41 | 64.33 | 0.70 | 28.34 |

| | | | | | |
|---|---|---|---|---|---|
| SE± | 1.23 | 1.54 | 1.65 | 0.32 | 1.12 |
| C.D. 0.05% | 3.7 | 4.59 | 4.97 | 0.96 | 3.37 |

### Conclusion

All the doses of Bio Insecticide reported significant improvement over water control in all the recorded parameters. The treatment Nutrozen reported maximum curd yield with morphology development than rest of the treatments. The Bio Insecticide @ 2 ml/lit. also noted maximum curd yield improvement over Double 0.5 ml/lit (FIG. 1).

### Example 9: Effect of Bio-Stimulant on Growth and Yield of Chickpea

| | |
|---|---|
| Name of the Product: Bio Insecticide | |
| Variety: Phule Vikram (MPKV) | Crop: Chickpea |

### Material and Method

A field experiment was conducted at Kay Bee R&D Farm, Phaltan (MS) during Rabi season 2022-2023. The experimental site under subtropical climatic condition. The experiment consisted of ten treatments as presented in Table 9. The chickpea variety Phule Vikram (MPKV) was used and experiment was arranged in Randomized Block Design with three replications. The net size of each plot was 5.00 m×3.60 m². All the recommended practices were applied as per stage and condition of crop. The spraying of the formulation was conducted at 35 and 55 Days After Sowing (DAS). The morphological data like plant height and no. of branches per plant were recorded at physiological maturity. The yield and yield contributing data were recorded as per the crop stage of occurrence. Collected data on different parameters were statistically analyzed by using “Analysis of Variance Method (ANOVA)” (Panse and Sukhatme, 1967).

### Results

The data on effect of bio-stimulant on growth and yield of chickpea were recorded statistically significant result and are furnished in Table 9.

TABLE 9
Response of Bio-stimulants on Chickpea

| | | | | Number | | 100- | |
|---|---|---|---|---|---|---|---|
| | | | Plant | of | Number | seed | Grain |
| Tr. | | Dose | Height | branches/ | of pods/ | weight | Yield |
| No. | Treatment. Code | (ml/lit.) | (cm) | plants | plants | (g) | (q/Ha.) |
| 1 | Bio Insecticide | 0.50 | 38.65 | 13.67 | 31.23 | 5.81 | 19.45 |
| 2 | Bio Insecticide | 1.00 | 38.73 | 14.33 | 32.47 | 5.86 | 19.87 |
| 3 | Bio Insecticide | 2.00 | 40.27 | 17.03 | 35.03 | 6.00 | 22.65 |
| 4 | Nutrozen (Plant derived | 2.00 | 43.23 | 16.67 | 36.23 | 6.21 | 23.03 |
| | nutrition along with the | | | | | | |
| | necessary hormones, | | | | | | |
| | vitamins and sea weed | | | | | | |
| | extract) | | | | | | |
| 5 | Isabion (Organic fraction | 2.00 | 45.38 | 17.83 | 37.00 | 6.34 | 23.12 |
| | derived from Amino Acid | | | | | | |
| | and peptides mixture | | | | | | |
| | 62.5%) | | | | | | |
| 6 | Biozyme Crop+ | 2.00 | 43.12 | 17.37 | 37.38 | 6.78 | 23.34 |
| | (Seaweed Extract, | | | | | | |
| | Proteins, Carbohydrates, | | | | | | |
| | Inorganic Salts and Other | | | | | | |
| | Inherent Nutrients) 22% | | | | | | |
| 7 | Double | 0.50 | 40.82 | 14.97 | 33.00 | 5.97 | 20.00 |
| | (Homobrassinolide | | | | | | |
| | 0.04%) | | | | | | |
| 8 | Macarena (Fermented | 1.25 | 39.51 | 15.77 | 33.57 | 6.05 | 20.11 |
| | extract Nutrient 15%) | | | | | | |
| 9 | Hoshi (Gibberellic Acid | 2.00 | 42.62 | 16.00 | 35.73 | 6.12 | 21.42 |
| | 0.001%) | | | | | | |
| 10 | Water Control | — | 37.38 | 13.00 | 29.37 | 5.75 | 18.43 |

| | | | | | |
|---|---|---|---|---|---|
| SE± | 1.45 | 0.76 | 1.21 | 0.23 | 1.01 |
| C.D. 0.05% | 4.35 | 2.27 | 3.65 | 0.67 | 3.03 |

### Conclusion

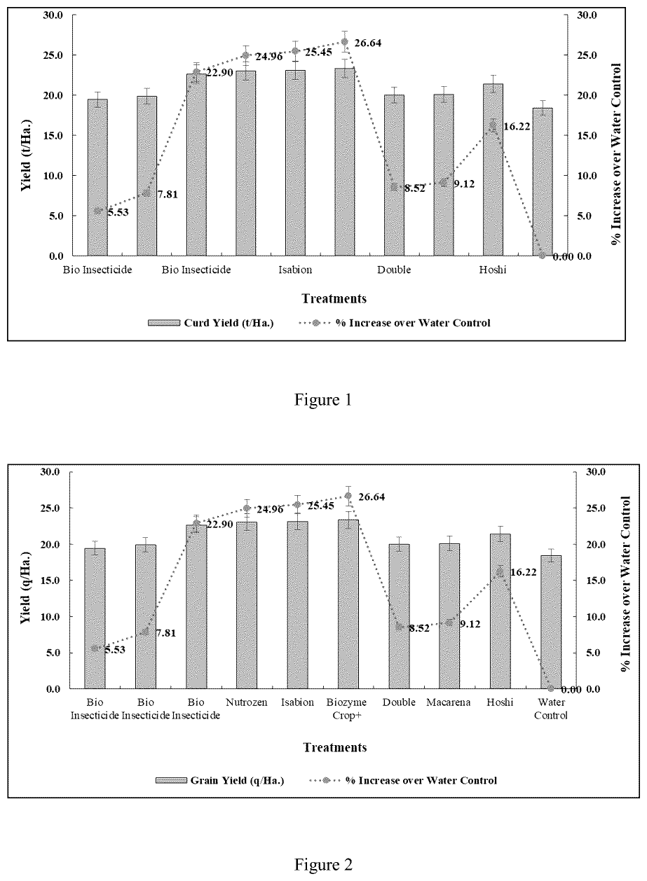

All the doses of Bio Insecticide reported significant improvement over water control in all the recorded parameters. The treatment Biozyme Crop+ reported maximum grain yield with morphology development than rest of the treatments. The Bio Insecticide @ 2 ml/lit. also noted maximum grain yield improvement over Double 0.5 ml/lit., Macarena 1.25 ml/lit. and Hoshi 2 ml/lit (FIG. 2).

### Example 10: Effect of Bio-Stimulant on Growth and Yield of Gerbera

| | |
|---|---|
| Name of the Product: Bio Insecticide | |
| Variety: Ruby Red | Crop: Gerbera |

### Material and Method

A field experiment was conducted at Kay Bee R&D Farm, Pune (MS) during Rabi season 2022-2023. The experimental site under sub-tropical climatic condition. The experiment consisted of ten treatments as presented in Table 10. The gerbera variety Ruby Red was used and experiment was arranged in Randomized Block Design with three replications. All the recommended practices were applied as per stage and condition of crop. The spraying of the formulation was started from 20 Days After Planting (DAT). Four sprays were taken at 25 days interval. The morphological data like number of leaves per plant and number of suckers per plant were recorded at physiological maturity. The yield and yield contributing data were recorded as per the crop stage of occurrence. Collected data on different parameters were statistically analyzed by using “Analysis of Variance Method (ANOVA)” (Panse and Sukhatme, 1967).

### Results

The data on effect of bio-stimulant on growth and yield of gerbera were recorded statistically significant result and are furnished in Table 10.

TABLE 10
Response of Bio-stimulants on Gerbera

| | | | No. of | No. of | Stalk | Flower | No. of |
|---|---|---|---|---|---|---|---|
| Tr. | | Dose | leaves/ | suckers/ | length | diameter | flowers/ |
| No. | Treatment. Code | (ml/lit.) | plant | plant | (cm) | (cm) | plant |
| 1 | Bio Insecticide | 0.50 | 26.10 | 3.47 | 28.14 | 9.90 | 8.30 |
| 2 | Bio Insecticide | 1.00 | 26.38 | 3.63 | 30.24 | 10.28 | 8.57 |
| 3 | Bio Insecticide | 2.00 | 30.65 | 4.23 | 34.67 | 11.34 | 9.42 |
| 4 | Nutrozen (Plant derived | 2.00 | 39.45 | 6.33 | 44.25 | 13.44 | 10.65 |
| | nutrition along with the | | | | | | |
| | necessary hormones, | | | | | | |
| | vitamins and sea weed | | | | | | |
| | extract) | | | | | | |
| 5 | Isabion (Organic fraction | 2.00 | 33.47 | 4.77 | 38.64 | 11.78 | 10.11 |
| | derived from Amino Acid | | | | | | |
| | and peptides mixture | | | | | | |
| | 62.5%) | | | | | | |
| 6 | Biozyme Crop+ | 2.00 | 38.25 | 5.83 | 43.12 | 12.90 | 10.44 |
| | (Seaweed Extract, | | | | | | |
| | Proteins, Carbohydrates, | | | | | | |
| | Inorganic Salts and Other | | | | | | |
| | Inherent Nutrients) 22% | | | | | | |
| 7 | Double | 0.50 | 28.40 | 3.97 | 31.58 | 10.87 | 9.12 |
| | (Homobrassinolide | | | | | | |
| | 0.04%) | | | | | | |
| 8 | Macarena (Fermented | 1.25 | 35.89 | 5.37 | 41.28 | 12.54 | 10.28 |
| | extract Nutrient 15%) | | | | | | |
| 9 | Hoshi (Gibberellic Acid | 2.00 | 40.13 | 6.87 | 46.73 | 13.87 | 10.7 |
| | 0.001%) | | | | | | |
| 10 | Water Control | — | 25.87 | 3.33 | 27.23 | 9.67 | 7.67 |

| | | | | | |
|---|---|---|---|---|---|
| SE± | 0.79 | 0.31 | 0.85 | 0.62 | 0.69 |
| C.D. 0.05% | 2.38 | 0.94 | 2.57 | 1.84 | 2.04 |

### Conclusion

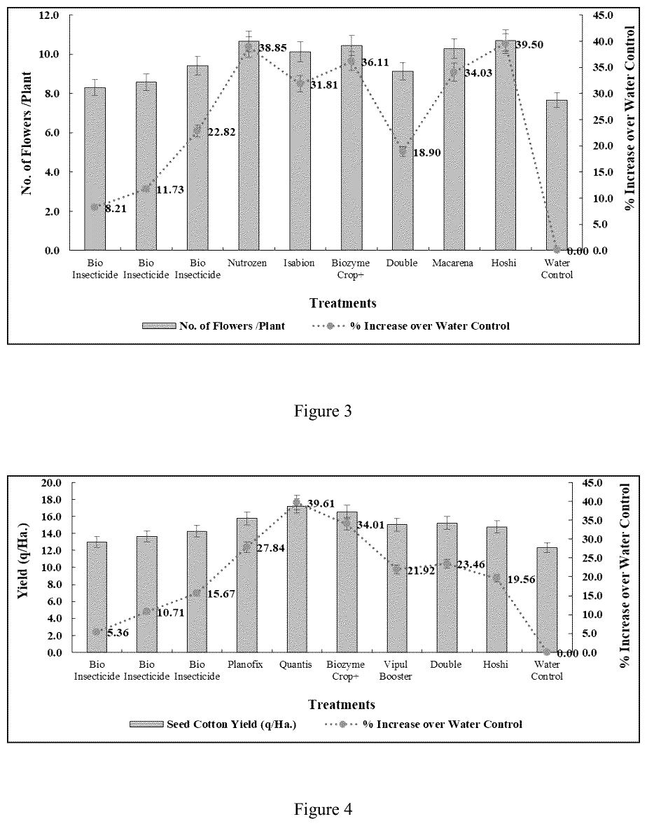

All the doses of Bio Insecticide reported significant improvement over water control in all the recorded parameters. The treatment Hoshi reported maximum yield with morphology development than rest of the treatments. The Bio Insecticide @ 2 ml/lit. also noted maximum yield improvement over Double 0.5 ml/lit (FIG. 3).

### Example 11: Effect of Bio-Stimulant on Growth and Yield of Cotton

| | |
|---|---|
| Name of the Product: Bio Insecticide | |
| Variety: HY Cotton-Ankur Pushkar BG-II | Crop: Cotton |

### Material and Method

A field experiment was conducted at Kay Bee R&D Farm, Phaltan (MS) during Kharif season 2021-2022. The experimental site under subtropical climatic condition. The experiment consisted of ten treatments as presented in Table 11. The Cotton variety Hy Cotton Ankur Pushkar BG-II was used and experiment was arranged in Randomized Block Design with three replications. The net size of each plot was 4.80 m×3.60 m². All the recommended practices were applied as per stage and condition of crop. The spraying of the formulation was conducted at 35 and 65 Days After Sowing (DAS). The morphological data like plant height, no. of sympodial branches was recorded at physiological maturity. The yield and yield contributing data were recorded as per the crop stage of occurrence. Collected data on different parameters were statistically analyzed by using “Analysis of Variance Method (ANOVA)” (Panse and Sukhatme, 1967).

### Results

The data on effect of bio-stimulant on growth and yield of cotton were recorded statistically significant result and are furnished in Table 11 and 12.

TABLE 11
Response of Bio-stimulants on Cotton.

| | | | | No. of | |
|---|---|---|---|---|---|
| | | | Plant | Sympodial | Days to |
| Tr. | Treatment | Dose | Height | Branches/ | 50% |
| No. | Code | (ml/lit.) | (cm) | plant | flowering |
| 1. | Bio Insecticide | 0.5 | 146.83 | 16.23 | 64.97 |
| 2. | Bio Insecticide | 1.0 | 147.00 | 17.00 | 64.70 |
| 3. | Bio Insecticide | 2.0 | 147.85 | 18.00 | 63.67 |
| 4. | Planofix (Alpha Napthyl Acetic | 0.4 | 147.13 | 18.37 | 61.63 |
| | Acid 4.5 Sl) | | | | |
| 5. | Quantis (Vinasse Metabolites | 2.0 | 157.42 | 20.33 | 61.00 |
| | Containing Amino Acids and | | | | |
| | Nutrients 52%) | | | | |
| 6. | Biozyme Crop+ (Seaweed Extract, | 2.0 | 153.59 | 20.00 | 62.37 |
| | Proteins, Carbohydrates, Inorganic | | | | |
| | Salts and Other Inherent Nutrients) | | | | |
| | 22% | | | | |
| 7. | Vipul Booster | 1.25 | 151.43 | 19.67 | 62.20 |
| | (Triacontanol 0.1%) | | | | |
| 8. | Double | 0.5 | 150.75 | 17.43 | 63.30 |
| | (Homobrassinolide 0.04%) | | | | |
| 9. | Hoshi (Gibberellic Acid 0.001%) | 2.0 | 154.89 | 18.77 | 63.00 |
| 10. | Water Control | — | 146.00 | 16.00 | 65.00 |

| | | | |
|---|---|---|---|
| SE± | 2.57 | 0.34 | 1.34 |
| C.D. 0.05% | 7.7 | 1.03 | 4.02 |

TABLE 12
Response of Bio-stimulants on Cotton.

| | | | | | Seed |
|---|---|---|---|---|---|
| | | | | Staple | Cotton |
| Tr. | Treatment | Dose | Single Boll | Length | Yield |
| No. | Code | (ml/lit.) | Weight (g) | (mm) | (q/Ha.) |
| 1. | Bio Insecticide | 0.5 | 4.36 | 23.03 | 12.98 |
| 2. | Bio Insecticide | 1.0 | 4.63 | 23.97 | 13.64 |
| 3. | Bio Insecticide | 2.0 | 4.89 | 24.56 | 14.25 |
| 4. | Planofix (Alpha Napthyl Acetic | 0.4 | 5.75 | 26.19 | 15.75 |
| | Acid 4.5 Sl) | | | | |
| 5. | Quantis (Vinasse Metabolites | 2.0 | 6.02 | 27.37 | 17.83 |
| | Containing Amino Acids and | | | | |
| | Nutrients 52%) | | | | |
| 6. | Biozyme Crop+ (Seaweed Extract, | 2.0 | 5.89 | 27.00 | 16.51 |
| | Proteins, Carbohydrates, Inorganic | | | | |
| | Salts and Other Inherent Nutrients) | | | | |
| | 22% | | | | |
| 7. | Vipul Booster (Triacontanol 0.1%) | 1.25 | 5.32 | 25.24 | 15.02 |
| 8. | Double | 0.5 | 5.68 | 26.87 | 15.21 |
| | (Homobrassinolide 0.04%) | | | | |
| 9. | Hoshi (Gibberellic Acid 0.001%) | 2.0 | 5.08 | 24.26 | 14.73 |
| 10. | Water Control | — | 4.12 | 22.43 | 12.32 |

| | | | |
|---|---|---|---|
| SE± | 0.13 | 0.48 | 1.03 |
| C.D. 0.05% | 0.4 | 1.44 | 3.08 |

### Conclusion

All the doses of Bio Insecticide reported significant improvement over water control in all the recorded parameters. The treatment Quantis reported maximum seed cotton yield. The Bio Insecticide @ 2 ml/lit. recorded maximum seed cotton yield improvement over water control. The Bio Insecticide @ 1 ml/lit. and 0.5 ml/lit. also noted improvement over water control (FIG. 4).

### Example 12: Effect of Bio-Stimulant on Yield of Guava

| | |
|---|---|
| Name of the Product: Bio Insecticide | |
| Variety: L-49 | Crop: Guava |

### Material and Method

A field experiment was conducted at Kay Bee R&D Farm, (MS) during Rabi season 2021-2022. The experimental site under sub-tropical climatic condition. The experiment consisted of ten treatments as presented in Table 13. The guava variety L-49 was used and experiment was arranged in Randomized Block Design with three replications. All the recommended practices were applied as per stage and condition of crop. The spraying of the formulation was conducted at 25 and 55 Days After Flowering (DAF). The yield and yield contributing data were recorded as per the crop stage of occurrence. Collected data on different parameters were statistically analyzed by using “analysis of variance method (ANOVA)” (Panse and Sukhatme, 1967).

### Results

The data on effect of bio-stimulant on growth and yield of guava were recorded statistically significant result and are furnished in Table 13.

TABLE 13
Response of Bio-stimulants on Guava

| | | | | | | Yield |
|---|---|---|---|---|---|---|
| | | | | Fruit | Fruit | per |
| Tr. | | Dose | Fruit | weight | diameter | tree |
| No. | Treatment. Code | (ml/lit.) | Set (%) | (g) | (cm) | (kg) |
| 1 | Bio Insecticide | 0.50 | 52.84 | 203.75 | 6.72 | 3.89 |
| 2 | Bio Insecticide | 1.00 | 59.36 | 217.32 | 7.03 | 4.10 |
| 3 | Bio Insecticide | 2.00 | 70.34 | 221.90 | 7.84 | 4.85 |
| 4 | Nutrozen (Plant derived | 2.00 | 75.76 | 236.27 | 8.12 | 5.08 |
| | nutrition along with the | | | | | |
| | necessary hormones, vitamins | | | | | |
| | and sea weed extract) | | | | | |
| 5 | Isabion (Organic fraction | 2.00 | 70.67 | 231.47 | 8.34 | 4.95 |
| | derived from Amino Acid and | | | | | |
| | peptides mixture 62.5%) | | | | | |
| 6 | Biozyme Crop+ (Seaweed | 2.00 | 63.56 | 226.56 | 8.05 | 4.65 |
| | Extract, Proteins, | | | | | |
| | Carbohydrates, Inorganic Salts | | | | | |
| | and Other Inherent Nutrients) | | | | | |
| | 22% | | | | | |
| 7 | Double (Homobrassinolide | 0.50 | 56.37 | 211.36 | 7.31 | 3.75 |
| | 0.04%) | | | | | |
| 00 8 | Macarena (Fermented extract | 1.25 | 65.30 | 214.23 | 7.45 | 4.35 |
| | Nutrient 15%) | | | | | |
| 9 | Fantac Plus (L-cysteine based | 1.50 | 73.83 | 225.67 | 8.11 | 5.02 |
| | plant growth regulator- | | | | | |
| | combination of amino acids and | | | | | |
| | vitamins) | | | | | |
| 10 | Water Control | — | 47.22 | 194.12 | 6.32 | 3.58 |

| | | | | |
|---|---|---|---|---|
| SE± | 2.43 | 0.46 | 1.23 | 0.47 |
| C.D. 0.05% | 7.29 | 1.37 | 3.7 | 1.43 |

### Conclusion

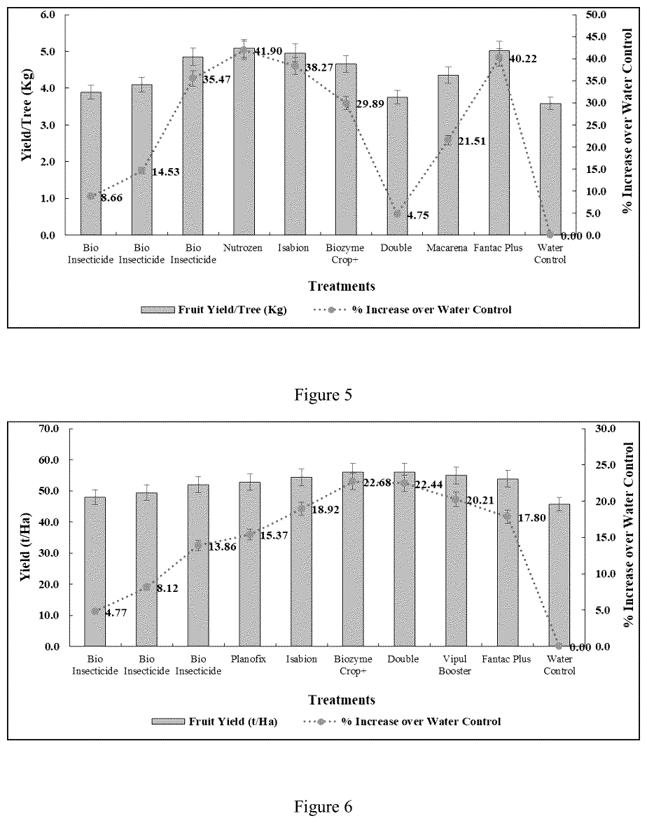

All the doses of Bio Insecticide reported significant improvement over water control in all the recorded parameters. The treatment Neutrozen reported maximum yield with morphology development than rest of the treatments. The Bio Insecticide @ 2 ml/lit. also noted maximum yield improvement over Biozyme Crop+2 ml/lit., Double 0.5 ml/lit. and Macarena 1.25 ml/lit (FIG. 5).

### Example 13: Effect of Bio-Stimulant on Growth and Yield of Tomato

| | |
|---|---|
| Name of the Product: Bio Insecticide | |
| Variety: INDUS 1030 | Crop: Tomato |

### Material and Method

A field experiment was conducted at Kay Bee R & D Farm, Phaltan (MS) during Rabi season 2022-2023. The experimental site under subtropical climatic condition. The experiment consisted of ten treatments as presented in Table 14. The Tomato variety Indus 1030 (Indus Seeds) was used and experiment was arranged in Randomized Block Design with three replications. The net size of each plot was 5.0×7.2 m². All the recommended practices were applied as per stage and condition of crop. The spraying of the formulation was conducted at 30, 60 and 90 Days After Transplanting (DAT). The morphological data like plant height and number of branches were recorded at physiological maturity. The data on days to flower initiation was recorded at the time of stage of occurrence. Number of flowers cluster was recorded at 120 DAT. The yield and yield contributing data were recorded as per the crop stage of occurrence. Collected data on different parameters were statistically analyzed by using “analysis of variance method (ANOVA)” (Panse and Sukhatme, 1967).

### Results

The data on effect of bio-stimulant on growth and yield of tomato were recorded statistically significant result and are furnished in Table 14.

TABLE 14
Response of Bio-stimulants on Tomato

| | | | | | | No. of | |
|---|---|---|---|---|---|---|---|
| | | | Plant | No. of | Days to | Flower | Fruit |
| Tr. | | Dose | Height | Branches/ | flower | Cluster/ | yield |
| No. | Treatment. Code | (ml/lit.) | (cm) | Plant | initiation | Plants | (t/ha) |
| 1 | Bio Insecticide | 0.50 | 89.56 | 12.33 | 39.00 | 14.30 | 47.85 |
| 2 | Bio Insecticide | 1.00 | 91.72 | 13.23 | 37.00 | 15.73 | 49.38 |
| 3 | Bio Insecticide | 2.00 | 97.37 | 14.67 | 34.87 | 16.30 | 52.00 |
| 4 | Planofix (Alpha Napthyl | 1.00 | 92.57 | 16.23 | 33.50 | 16.87 | 52.69 |
| | Acetic Acid 4.5 Sl) | | | | | | |
| 5 | Isabion (Organic fraction | 2.00 | 100.65 | 17.40 | 34.67 | 17.00 | 54.31 |
| | derived from Amino Acid | | | | | | |
| | and peptides mixture | | | | | | |
| | 62.5%) | | | | | | |
| 6 | Biozyme Crop+ | 2.00 | 102.32 | 16.00 | 34.00 | 17.43 | 56.03 |
| | (Seaweed Extract, | | | | | | |
| | Proteins, Carbohydrates, | | | | | | |
| | Inorganic Salts and Other | | | | | | |
| | Inherent Nutrients) 22% | | | | | | |
| 7 | Double | 0.50 | 94.29 | 15.20 | 35.63 | 15.87 | 55.92 |
| | (Homobrassinolide | | | | | | |
| | 0.04%) | | | | | | |
| 8 | Vipul Booster | 1.25 | 98.36 | 14.00 | 36.60 | 16.37 | 54.90 |
| | (Triacontanol 0.1%) | | | | | | |
| 9 | Fantac Plus (L-cysteine | 1.50 | 103.83 | 14.03 | 37.80 | 16.70 | 53.80 |
| | based plant growth | | | | | | |
| | regulator-combination of | | | | | | |
| | amino acids and | | | | | | |
| | vitamins) | | | | | | |
| 10 | Water Control | — | 87.34 | 11.30 | 39.33 | 13.57 | 45.67 |

| | | | | | |
|---|---|---|---|---|---|
| SE± | 1.65 | 0.11 | 0.21 | 0.09 | 1.10 |
| C.D. 0.05% | 4.95 | 0.33 | 0.62 | 0.28 | 3.30 |

### Conclusion

All the doses of Bio Insecticide reported significant improvement over water control in all the recorded parameters. The treatment Biozyme Crop+ reported maximum yield with morphology development than rest of the treatments. The Bio Insecticide @ 2 ml/lit. also noted maximum yield improvement over water control (FIG. 6).

### Example 14: Effect of Bio-Stimulant on Growth and Yield of Maize

| | | |
|---|---|---|
| Name of the Product: Bio Insecticide | | |
| Variety: P-3388 (PIONEER) | \grave{ } | Crop: Maize |

### Material and Method

A field experiment was conducted at Kay Bee R & D Farm, Gursale (MS) during kharif season 2021-2022. The experimental site under subtropical climatic condition. The experiment consisted of ten treatments as presented in Table 15. The Maize variety P-3388 (Pioneer) was used and experiment was arranged in Randomized Block Design with three replications. The net size of each plot was 5.0×7.0 m². All the recommended practices were applied as per stage and condition of crop. The spraying of the formulation was conducted at 25, 45 and 65 Days After Sowing (DAS). The morphological data like plant height and leaf area index were recorded at physiological maturity. The yield and yield contributing data were recorded as per the crop stage of occurrence. Collected data on different parameters were statistically analyzed by using “analysis of variance method (ANOVA)” (Panse and Sukhatme, 1967).

### Results

The data on effect of bio-stimulant on growth and yield of maize were recorded statistically significant result and are furnished in Table 15.

TABLE 15
Response of Bio-stimulants on Maize.

| | | | | | | 1000 | |
|---|---|---|---|---|---|---|---|
| | | | Plant | Leaf | Cob | Grains | Grain |
| | | Dose | Height | Area | Length | Weight | Yield |
| Tr. No. | Treatment. Code | (ml/lit.) | (cm) | Index | (cm) | (g) | (q/Ha.) |
| 1 | Bio Insecticide | 0.50 | 150.21 | 6.12 | 16.55 | 26.42 | 41.54 |
| 2 | Bio Insecticide | 1.00 | 161.60 | 6.46 | 17.54 | 26.93 | 43.48 |
| 3 | Bio Insecticide | 2.00 | 163.94 | 6.56 | 18.06 | 27.11 | 45.52 |
| 4 | Nutrozen (Plant derived | 2.00 | 197.65 | 8.09 | 21.97 | 28.93 | 56.2 |
| | nutrition along with the | | | | | | |
| | necessary hormones, | | | | | | |
| | vitamins and sea weed | | | | | | |
| | extract) | | | | | | |
| 5 | Isabion (Organic fraction | 2.00 | 194.25 | 7.91 | 22.38 | 28.62 | 55.1 |
| | derived from Amino Acid | | | | | | |
| | and peptides mixture | | | | | | |
| | 62.5%) | | | | | | |
| 6 | Biozyme Crop+ | 2.00 | 176.81 | 7.07 | 19.48 | 27.62 | 51.25 |
| | (Seaweed Extract, | | | | | | |
| | Proteins, Carbohydrates, | | | | | | |
| | Inorganic Salts and Other | | | | | | |
| | Inherent Nutrients) 22% | | | | | | |
| 7 | Double | 0.50 | 159.22 | 6.37 | 17.80 | 26.75 | 44.9 |
| | (Homobrassinolide | | | | | | |
| | 0.04%) | | | | | | |
| 8 | Macarena (Fermented | 1.25 | 187.58 | 7.50 | 20.66 | 28.09 | 54.37 |
| | extract Nutrient 15%) | | | | | | |
| 9 | Hoshi (Gibberellic Acid | 2.00 | 174.40 | 6.94 | 19.10 | 27.44 | 48.26 |
| | 0.001%) | | | | | | |
| 10 | Water Control | — | 140.48 | 5.46 | 13.82 | 25.49 | 39.4 |

| | | | | | |
|---|---|---|---|---|---|
| SE± | 1.98 | 0.12 | 0.41 | 0.58 | 1.12 |
| C.D. 0.05% | 5.97 | 0.38 | 1.24 | 1.76 | 3.37 |

### Conclusion

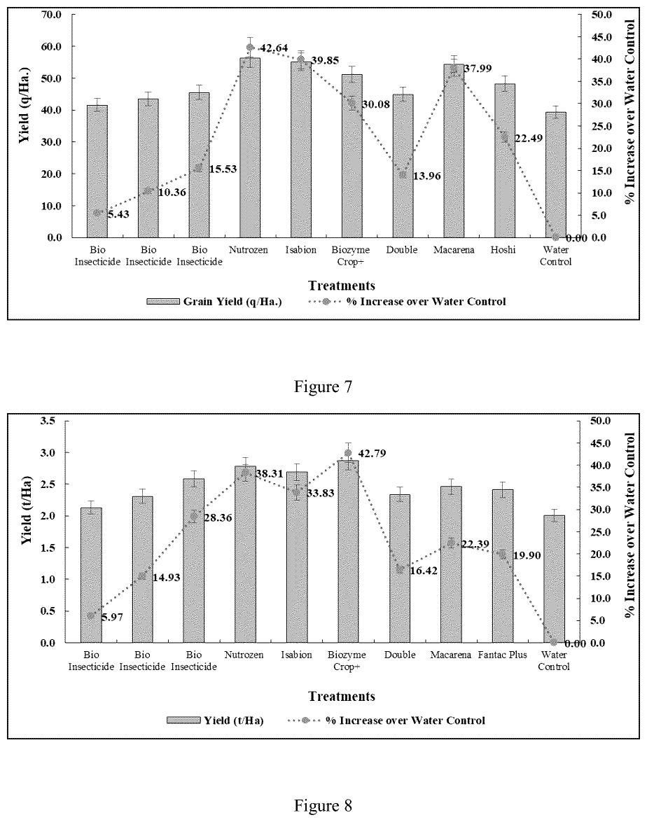

All the doses of Bio Insecticide reported significant improvement over water control in all the recorded parameters. The treatment Neutrozen reported maximum yield with morphology development than rest of the treatments. The Bio Insecticide@ 2 ml/lit. also noted maximum yield improvement over Double 0.5 ml/lit (FIG. 7).

### Example 15: Effect of Bio-Stimulant on Growth and Yield of Mustard

| | |
|---|---|
| Name of the Product: Bio Insecticide | |
| Variety: DRMR 1165-40 | Crop: Mustard |

### Material and Method

A field experiment was conducted at Kay Bee R & D Farm, Phaltan, (MS) during Rabi season 2021-2022. The experimental site under tropical climatic condition. The experiment consisted of ten treatments as presented in Table 16. The Mustard variety DRMR 1165-40 was used and experiment was arranged in Randomized Block Design with three replications. The net size of each plot was 2.0 m×2.0 m². All the recommended practices were applied as per stage and condition of crop. The spraying of the formulation was conducted at 25 and 45 Days After Sowing (DAS). The morphological data like plant height and number of branches per plant were recorded at physiological maturity. The yield and yield contributing data were recorded as per the crop stage of occurrence. The oil content was estimated by using Soxhlet method after harvesting. Collected data on different parameters were statistically analyzed by using “analysis of variance method (ANOVA)” (Panse and Sukhatme, 1967).

### Results

The data on effect of bio-stimulant on growth and yield of mustard were recorded statistically significant result and are furnished in Table 16.

TABLE 16
Response of Bio-stimulants on Mustard.

| | | | | Number | | 1000- | | |
|---|---|---|---|---|---|---|---|---|
| | | | Plant | of | Siliqua/ | seed | | Oil |
| Tr. | | Dose | Height | Branches/ | plant | weight | Yield | Content |
| No. | Treatment. Code | (ml/lit.) | (cm) | Plant | (no.) | (g) | (t/ha.) | (%) |
| 1 | Bio Insecticide | 0.50 | 170.12 | 4.43 | 59.43 | 2.26 | 2.13 | 39.87 |
| 2 | Bio Insecticide | 1.00 | 175.34 | 4.50 | 61.00 | 2.30 | 2.31 | 40.02 |
| 3 | Bio Insecticide | 2.00 | 189.61 | 4.93 | 67.20 | 2.58 | 2.58 | 41.34 |
| 4 | Nutrozen (Plant | 2.00 | 193.26 | 5.37 | 70.23 | 2.69 | 2.78 | 43.25 |
| | derived nutrition | | | | | | | |
| | along with the | | | | | | | |
| | necessary | | | | | | | |
| | hormones, | | | | | | | |
| | vitamins and sea | | | | | | | |
| | weed extract) | | | | | | | |
| 5 | Isabion (Organic | 2.00 | 197.78 | 5.63 | 69.00 | 2.52 | 2.69 | 41.78 |
| | fraction derived | | | | | | | |
| | from Amino Acid | | | | | | | |
| | and peptides | | | | | | | |
| | mixture 62.5%) | | | | | | | |
| 6 | Biozyme Crop+ | 2.00 | 195.37 | 5.00 | 73.53 | 2.54 | 2.87 | 42.36 |
| | (Seaweed Extract, | | | | | | | |
| | Proteins, | | | | | | | |
| | Carbohydrates, | | | | | | | |
| | Inorganic Salts | | | | | | | |
| | and Other | | | | | | | |
| | Inherent | | | | | | | |
| | Nutrients) 22% | | | | | | | |
| 7 | Double | 0.50 | 184.98 | 4.80 | 61.57 | 2.38 | 2.34 | 38.45 |
| | (Homobrassinolide | | | | | | | |
| | 0.04%) | | | | | | | |
| 8 | Macarena | 1.25 | 181.93 | 4.73 | 64.60 | 2.46 | 2.46 | 40.54 |
| | (Fermented | | | | | | | |
| | extract Nutrient | | | | | | | |
| | 15%) | | | | | | | |
| 9 | Fantac Plus (L- | 1.50 | 176.37 | 4.83 | 63.47 | 2.43 | 2.41 | 40.23 |
| | cysteine based | | | | | | | |
| | plant growth | | | | | | | |
| | regulator- | | | | | | | |
| | combination of | | | | | | | |
| | amino acids and | | | | | | | |
| | vitamins) | | | | | | | |
| 10 | Water Control | — | 167.02 | 4.33 | 57.30 | 2.18 | 2.01 | 37.24 |

| | | | | | | |
|---|---|---|---|---|---|---|
| SE± | 2.43 | 0.46 | 1.23 | 0.47 | 0.52 | 1.32 |
| C.D. 0.05% | 7.29 | 1.37 | 3.7 | 1.43 | 1.57 | 3.97 |

### Conclusion

All the doses of Bio Insecticide reported significant improvement over water control in all the recorded parameters. The treatment Biozyme Crop+ reported maximum yield with morphology development than rest of the treatments. The Bio Insecticide @ 2 ml/lit. also noted maximum yield improvement over Double 0.5 ml/lit., Macarena 1.25 ml/lit. and Fantac Plus 1.50 ml/lit (FIG. 8).

### Example 16: Effect of Bio-Stimulant on Growth and Yield of Rose

| | |
|---|---|
| Name of the Product: Bio Insecticide | |
| Variety: Revival | Crop: Rose |

### Material and Method

A field experiment was conducted at Kay Bee R&D Farm, Phaltan (MS) during Rabi season 2022-2023. The experimental site under subtropical climatic condition. The experiment consisted of ten treatments as presented in Table 17. The Rose variety Rivival was used and experiment was arranged in Randomized Block Design with three replications. All the recommended practices were applied as per stage and condition of crop. The spraying of the formulation was conducted at 20 and 40 Days After Pruning (DAP). The morphological data like plant height, number of branches and flower stem length were recorded at physiological maturity. The yield and yield contributing data were recorded as per the crop stage of occurrence. Collected data on different parameters were statistically analyzed by using “analysis of variance method (ANOVA)” (Panse and Sukhatme, 1967).

### Results

The data on effect of bio-stimulant on growth and yield of rose were recorded statistically significant result and are furnished in Table 17.

TABLE 17
Response of Bio-stimulants on Rose

| | | | | | Flower | Flower | | Yield: |
|---|---|---|---|---|---|---|---|---|
| | | | Plant | Number | Stem | Stem | Vase | No. of |
| Tr. | Treatment. | Dose | Height | of | Length | Diameter | Life | Stems/M²/ |
| No. | Code | (ml/lit.) | (cm) | Branches | (cm) | (mm) | (Days) | Year |
| 1 | Bio Insecticide | 0.50 | 104.78 | 4.00 | 63.29 | 5.63 | 6.50 | 86.52 |
| 2 | Bio Insecticide | 1.00 | 109.90 | 4.13 | 67.10 | 5.96 | 6.60 | 90.48 |
| 3 | Bio Insecticide | 2.00 | 110.41 | 5.33 | 76.00 | 6.72 | 7.40 | 97.83 |
| 4 | Nutrozen (Plant | 2.00 | 114.41 | 6.13 | 76.45 | 7.03 | 7.67 | 103.52 |
| | derived nutrition | | | | | | | |
| | along with the | | | | | | | |
| | necessary | | | | | | | |
| | hormones, | | | | | | | |
| | vitamins and sea | | | | | | | |
| | weed extract) | | | | | | | |
| 5 | Isabion (Organic | 2.00 | 111.83 | 5.77 | 72.47 | 6.99 | 7.34 | 105.42 |
| | fraction derived | | | | | | | |
| | from Amino | | | | | | | |
| | Acid and | | | | | | | |
| | peptides mixture | | | | | | | |
| | 62.5%) | | | | | | | |
| 6 | Biozyme Crop+ | 2.00 | 112.35 | 6.07 | 77.47 | 7.83 | 7.43 | 108.78 |
| | (Seaweed | | | | | | | |
| | Extract, Proteins, | | | | | | | |
| | Carbohydrates, | | | | | | | |
| | Inorganic Salts | | | | | | | |
| | and Other | | | | | | | |
| | Inherent | | | | | | | |
| | Nutrients) 22% | | | | | | | |
| 7 | Double | 0.50 | 104.03 | 5.00 | 69.73 | 5.76 | 7.07 | 106.57 |
| | (Homobrassinolide | | | | | | | |
| | 0.04%) | | | | | | | |
| 8 | Macarena | 1.25 | 108.45 | 4.57 | 75.23 | 6.79 | 7.63 | 109.41 |
| | (Fermented | | | | | | | |
| | extract Nutrient | | | | | | | |
| | 15%) | | | | | | | |
| 9 | Hoshi | 2.00 | 113.78 | 6.47 | 79.42 | 7.42 | 8.53 | 110.35 |
| | (Gibberellic Acid | | | | | | | |
| | 0.001%) | | | | | | | |
| 10 | Water Control | — | 99.34 | 3.77 | 59.54 | 5.42 | 6.03 | 84.56 |

| | | | | | | |
|---|---|---|---|---|---|---|
| SE± | 1.90 | 0.23 | 1.45 | 0.39 | 0.54 | 1.99 |
| C.D. 0.05% | 5.71 | 0.70 | 4.35 | 1.17 | 1.63 | 5.97 |

### Conclusion

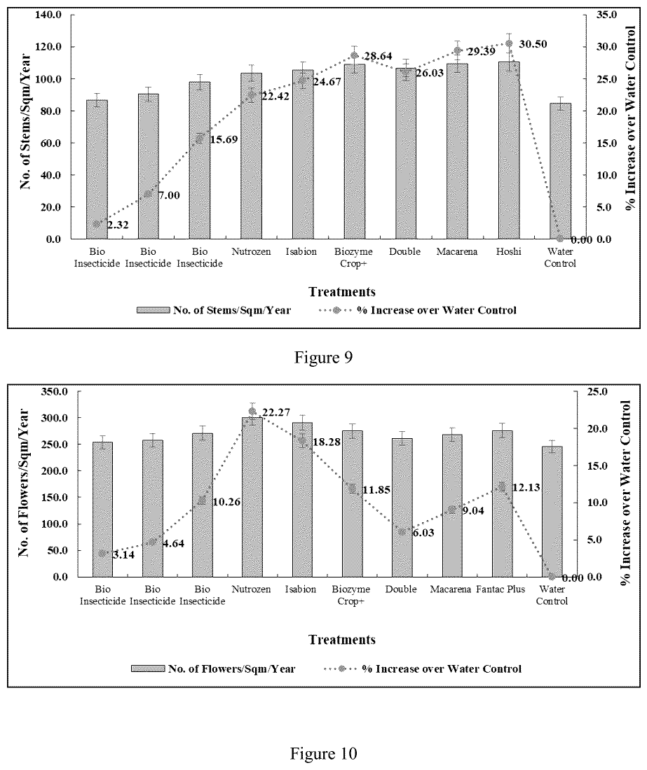

All the doses of Bio Insecticide reported significant improvement over water control in all the recorded parameters. The treatment Hoshi reported maximum yield with morphology development than rest of the treatments. The Bio Insecticide @ 2 ml/lit. Also, noted maximum yield improvement over water control (FIG. 9).

### Example 17: Effect of Bio-Stimulant on Yield of Carnation

| | |
|---|---|
| Name of the Product: Bio Insecticide | |
| Variety: PINK DONA | Crop: Carnation |

A field experiment was conducted at Kay Bee R & D Farm, Pune (MS) during Rabi season 2021-2022. The experimental site under tropical climatic condition. The experiment consisted of ten treatments as presented in Table 18. The carnation variety Pink Dona was used and experiment was arranged in Randomized Block Design with three replications. The net size of each plot was 5.0×1.5 m². All the recommended practices were applied as per stage and condition of crop. The spraying of the formulation was conducted at 20 Days interval after One month of Planting (DAP). The morphological data like flower stalk length, flower head diameter, calyx splitting and no. of flowering were recorded at physiological maturity. The yield and yield contributing data were recorded as per the crop stage of occurrence. Collected data on different parameters were statistically analyzed by using “analysis of variance method (ANOVA)” (Panse and Sukhatme, 1967).

### Results

The data on effect of bio-stimulant on growth and yield of carnation were recorded statistically significant result and are furnished in Table 18.

TABLE 18
Response of Bio-stimulants on Carnation.

| | | | Flower | Flower | | |
|---|---|---|---|---|---|---|
| | | | stalk | head | Calyx | Number of |
| Tr. | | Dose | length | diameter | splitting | flowers per |
| No. | Treatment. Code | (ml/lit.) | (cm) | (mm) | (%) | sqm/year |
| 1 | Bio Insecticide | 0.50 | 81.02 | 21.37 | 23.37 | 253.30 |
| 2 | Bio Insecticide | 1.00 | 82.26 | 22.01 | 17.83 | 257.00 |
| 3 | Bio Insecticide | 2.00 | 83.02 | 22.23 | 11.33 | 270.80 |
| 4 | Nutrozen (Plant derived | 2.00 | 85.23 | 23.02 | 9.67 | 300.30 |
| | nutrition along with the | | | | | |
| | necessary hormones, | | | | | |
| | vitamins and sea weed | | | | | |
| | extract) | | | | | |
| 5 | Isabion (Organic | 2.00 | 83.45 | 23.43 | 10.50 | 290.50 |
| | fraction derived from | | | | | |
| | Amino Acid and peptides | | | | | |
| | mixture 62.5%) | | | | | |
| 6 | Biozyme Crop+ | 2.00 | 84.78 | 22.56 | 12.43 | 274.70 |
| | (Seaweed Extract, | | | | | |
| | Proteins, Carbohydrates, | | | | | |
| | Inorganic Salts and Other | | | | | |
| | Inherent Nutrients) 22% | | | | | |
| 7 | Double | 0.50 | 81.47 | 22.06 | 15.73 | 260.40 |
| | (Homobrassinolide | | | | | |
| | 0.04%) | | | | | |
| 8 | Macarena (Fermented | 1.25 | 84.23 | 23.21 | 20.87 | 267.80 |
| | extract Nutrient 15%) | | | | | |
| 9 | Fantac Plus (L-cysteine | 1.50 | 82.89 | 22.21 | 18.93 | 275.40 |
| | based plant growth | | | | | |
| | regulator-combination of | | | | | |
| | amino acids and | | | | | |
| | vitamins) | | | | | |
| 10 | Water Control | — | 80.12 | 21.00 | 28.37 | 245.60 |

| | | | | |
|---|---|---|---|---|
| SE± | 2.13 | 1.12 | 1.09 | 3.45 |
| C.D. 0.05% | 6.38 | 3.36 | 3.28 | 10.36 |

### Conclusion

All the doses of Bio Insecticide reported significant improvement over water control in all the recorded parameters. The treatment Neutrozen reported maximum yield with morphology development than rest of the treatments. The Bio Insecticide @ 2 ml/lit. also noted maximum yield improvement over Double 0.5 ml/lit. and Macarena 1.25 ml/lit (FIG. 10).

### Example 18: Effect of Bio-Stimulant on Growth and Yield of Maize

| | |
|---|---|
| Name of the Product: Bio Insecticide | |
| Variety: Pioneer - P-3338 | Crop: Maize |

### Material and Method

A field experiment was conducted at Kay Bee R&D Farm, Gursale (MS) during Kharif season 2021-2022. The experimental site under subtropical climatic condition. The experiment consisted of ten treatments as presented in Table 19. The Maize variety Pioneer-P-3338 was used and experiment was arranged in Randomized Block Design with three replications. The net size of each plot was 5.0×3.6 m². All the recommended practices were applied as per stage and condition of crop. The spraying of the formulation was conducted at 25, 45 and 65 Days After Sowing (DAS). The morphological data like plant height and leaf area index were recorded at physiological maturity. The yield and yield contributing data were recorded as per the crop stage of occurrence. Collected data on different parameters were statistically analyzed by using “analysis of variance method (ANOVA)” (Panse and Sukhatme, 1967).

### Results

The data on effect of bio-stimulant on growth and yield of maize were recorded statistically significant result and are furnished in Table 19.

TABLE 19
Response of Bio-stimulants on Maize

| | | | | | | 1000 | |
|---|---|---|---|---|---|---|---|
| | | | Plant | Leaf | Cob | Grains | Grain |
| Tr. | | Dose | Height | Area | Length | Weight | Yield |
| No. | Treatment. Code | (ml/lit.) | (cm) | Index | (cm) | (g) | (q/ha.) |
| 1 | Bio Insecticide | 0.50 | 147.40 | 5.01 | 13.03 | 26.12 | 42.20 |
| 2 | Bio Insecticide | 1.00 | 165.80 | 6.66 | 16.33 | 27.33 | 43.56 |
| 3 | Bio Insecticide | 2.00 | 177.44 | 7.40 | 20.12 | 28.30 | 51.24 |
| 4 | Nutrozen (Plant derived | 2.00 | 192.80 | 8.33 | 21.44 | 29.30 | 54.31 |
| | nutrition along with the | | | | | | |
| | necessary hormones, | | | | | | |
| | vitamins and sea weed | | | | | | |
| | extract) | | | | | | |
| 5 | Isabion (Organic fraction | 2.00 | 180.70 | 8.10 | 22.93 | 28.44 | 53.10 |
| | derived from Amino Acid | | | | | | |
| | and peptides mixture 62.5%) | | | | | | |
| 6 | Biozyme Crop+ (Seaweed | 2.00 | 199.90 | 9.03 | 19.33 | 30.12 | 55.10 |
| | Extract, Proteins, | | | | | | |
| | Carbohydrates, Inorganic | | | | | | |
| | Salts and Other Inherent | | | | | | |
| | Nutrients) 22% | | | | | | |
| 7 | Double (Homobrassinolide | 0.50 | 175.67 | 7.50 | 15.40 | 26.80 | 46.10 |
| | 0.04%) | | | | | | |
| 8 | Macarena (Fermented | 1.25 | 178.01 | 7.90 | 18.33 | 27.40 | 49.33 |
| | extract Nutrient 15%) | | | | | | |
| 9 | Hoshi (Gibberellic Acid | 2.00 | 201.13 | 9.37 | 19.30 | 28.40 | 52.31 |
| | 0.001%) | | | | | | |
| 10 | Water Control | — | 145.43 | 5.13 | 12.34 | 25.73 | 39.45 |

| | | | | | |
|---|---|---|---|---|---|
| SE± | 1.43 | 0.45 | 0.10 | 0.76 | 1.23 |
| C.D. 0.05% | 4.28 | 1.34 | 0.31 | 2.29 | 3.70 |

### Conclusion

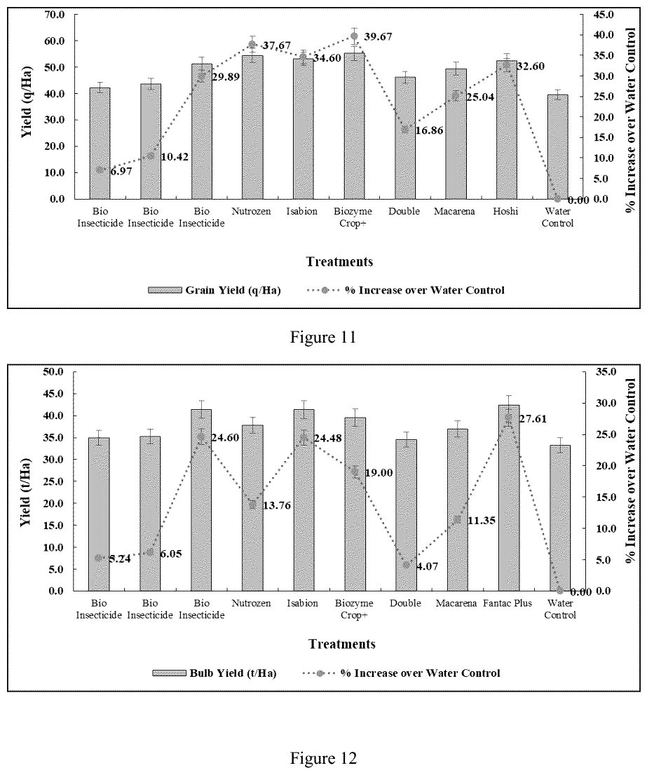

All the doses of Bio Insecticide reported significant improvement over water control in all the recorded parameters. The treatment Biozyme Crop+ reported maximum yield with morphology development than rest of the treatments. The Bio Insecticide @ 2 ml/lit. also noted maximum yield improvement over Double 0.5 ml/lit. and Macarena 1.25 ml/lit (FIG. 11).

### Example 19: Effect of Bio-Stimulant on Growth and Yield of Onion

| | |
|---|---|
| Name of the Product: Bio Insecticide | |
| Variety: BHIMA SUPER | Crop: Onion |

### Material and Method

A field experiment was conducted at Kay Bee R&D Farm, Phaltan (MS) during Rabi season 2021-2022. The experimental site under subtropical climatic condition. The experiment consisted of ten treatments as presented in Table 20. The onion variety Bhima Super was used and experiment was arranged in Randomized Block Design with three replications. The net size of each plot was 4.00×3.20 m². All the recommended practices were applied as per stage and condition of crop. The spraying of the formulation was conducted at 25, 45 and 65 Days After Transplanting (DAT). The morphological data like plant height and number of leaves per plant were recorded at physiological maturity. The yield and yield contributing data were recorded as per the crop stage of occurrence. Collected data on different parameters were statistically analyzed by using “analysis of variance method (ANOVA)” (Panse and Sukhatme, 1967).

### Results

The data on effect of bio-stimulant on growth and yield of Onion were recorded statistically significant result and are furnished in Table 20.

TABLE 20
Response of Bio-stimulants on Onion

| | | | Plant | | Bulb | | Bulb |
|---|---|---|---|---|---|---|---|
| Tr. | | Dose | Height | Number of | Diameter | Days to | Yield |
| No. | Treatment. Code | (ml/lit.) | (cm) | leaves/Plant | (cm) | Maturity | (t/ha.) |
| 1 | Bio Insecticide | 0.50 | 64.87 | 13.40 | 6.45 | 120.67 | 34.95 |
| 2 | Bio Insecticide | 1.00 | 68.50 | 13.43 | 6.59 | 119.00 | 35.22 |
| 3 | Bio Insecticide | 2.00 | 73.29 | 13.50 | 7.23 | 117.63 | 41.38 |
| 4 | Nutrozen (Plant | 2.00 | 72.10 | 13.43 | 7.14 | 118.60 | 37.78 |
| | derived nutrition | | | | | | |
| | along with the | | | | | | |
| | necessary hormones, | | | | | | |
| | vitamins and sea | | | | | | |
| | weed extract) | | | | | | |
| 5 | Isabion (Organic | 2.00 | 75.37 | 13.20 | 7.60 | 116.43 | 41.34 |
| | fraction derived | | | | | | |
| | from Amino Acid | | | | | | |
| | and peptides mixture | | | | | | |
| | 62.5%) | | | | | | |
| 6 | Biozyme Crop+ | 2.00 | 72.60 | 13.60 | 7.93 | 116.83 | 39.52 |
| | (Seaweed Extract, | | | | | | |
| | Proteins, | | | | | | |
| | Carbohydrates, | | | | | | |
| | Inorganic Salts and | | | | | | |
| | Other Inherent | | | | | | |
| | Nutrients) 22% | | | | | | |
| 7 | Double | 0.50 | 67.58 | 13.57 | 7.02 | 119.80 | 34.56 |
| | (Homobrassinolide | | | | | | |
| | 0.04%) | | | | | | |
| 8 | Macarena | 1.25 | 69.35 | 13.27 | 6.56 | 119.00 | 36.98 |
| | (Fermented extract | | | | | | |
| | Nutrient 15%) | | | | | | |
| 9 | Fantac Plus (L- | 1.50 | 74.21 | 14.00 | 7.64 | 115.37 | 42.38 |
| | cysteine based plant | | | | | | |
| | growth regulator- | | | | | | |
| | combination of | | | | | | |
| | amino acids and | | | | | | |
| | vitamins) | | | | | | |
| 10 | Water Control | — | 62.94 | 12.97 | 6.37 | 121.43 | 33.21 |

| | | | | | |
|---|---|---|---|---|---|
| SE± | 1.34 | 0.34 | 0.45 | 1.23 | 2.12 |
| C.D. 0.05% | 4.00 | NS | 1.37 | 3.68 | 6.36 |

### Conclusion

All the doses of Bio Insecticide reported significant improvement over water control in all the recorded parameters. The treatment Fantac Plus reported maximum yield with morphology development than rest of the treatments. The Bio Insecticide @ 2 ml/lit. also noted maximum yield improvement over Biozyme Crop+2 ml/lit., Double 0.5 ml/lit., Macarena 1.25 ml/lit (FIG. 12).

### Example 20: Bio-Efficacy of Bio-Insecticide Against DBM in Cauliflower

| | |
|---|---|
| Name of the Product: Bio-Insecticide | Variety: Kimaya |
| Crop: Cauliflower | Pest: DBM (Plutella xylostella) |

### Material and Method

A field experiment to study bio-efficacy of Bio-Insecticide in Cauliflower was conducted at Kay-Bee Bio-Organics Pvt. Ltd.'s farm at Phaltan, District Satara (M. S.), India during 2021-2022. The experiment comprising of twelve treatments was laid out in a Randomized Block Design (RBD) with three replications following spacings of 60 and 45 cm between rows and plants respectively. Each treatment consisted of an area of 5×4 m. Recommended agronomic practices were followed raise good crop. Observations on DBM larva population were taken on randomly selected five plants. Two sprays were taken pest population was recorded as per standard method in use before and after the application of Bio-Insecticide. The data were calculated by using standard statistical methods.

$$Reduction over control ( \% ) = \frac{\begin{matrix}Population Count of Control Plot - \\ Population Count of Treated Plot\end{matrix}}{Population Count of Control Plot} * 100$$

TABLE 21

| Tr. | | Dose ml or | |
|---|---|---|---|
| No. | Treatments | gm/L | |
| T 1 | Bio-Insecticide | 0.5 | ml |
| T 2 | Bio-Insecticide | 1 | ml |
| T 3 | Bio-Insecticide | 2 | ml |
| T 4 | Deltamethrin 2.8 EC (2.8% w/w) | 1.5 | ml |
| T 5 | Thiodicarb 75% WP | 2 | gm |
| T 6 | Spinosad 480 SC (45% w/w) | 0.5 | ml |
| T 7 | Chlorantraniliprole 18.5% SC | 0.3 | ml |
| T 8 | Spinetoram 11.7% SC | 0.9 | ml |
| T 9 | Emamectin Benzonate 5% SG | 0.4 | gm |
| T 10 | Chlorantraniliprole(10%) + | 0.5 | ml |
| | Lambdacyhalothrin(5%) ZC | | |
| T 11 | Cypermethrin 25% EC | 1 | ml |
| T 12 | Control | | |

### Results: The Results are Depicted in Table 22

TABLE 22

| | | | | | | | 2^{nd} spray | | | |
|---|---|---|---|---|---|---|---|---|---|---|
| | | | | | | | | % | | |
| | | | | | 1^{st} spray | | | Reduction | | |
| | | | | | | % | | in | | |
| | | | | | | Reduction | | DBM | | % |
| | | | | | No. of | in DBM | No. of | Population | | Increase |
| | | | | No. of | DBM | Population | DBM | (2nd | | in |
| | | Dose | | DBM | after | (1st spray) | after | spray) | | Yield |
| Tr. | | ml or | | before | 1st | over | 2nd | over | Yield | over |
| No. | Treatments | gm/L | | spray | spray | control | spray | control | (t/ha) | control |
| T 1 | Bio- | 0.5 | ml | 7.12 | 2.04 | 85.54 | 2.1 | 88.61 | 27.12 | 12.43 |
| | Insecticide | | | | | | | | | |
| T 2 | Bio- | 1 | ml | 8.24 | 1.56 | 88.94 | 1.58 | 91.43 | 28.48 | 16.61 |
| | Insecticide | | | | | | | | | |
| T 3 | Bio- | 2 | ml | 6.22 | 1.3 | 90.79 | 1.32 | 92.84 | 30.04 | 20.94 |
| | Insecticide | | | | | | | | | |
| T 4 | Deltamethrin | 1.5 | ml | 9.28 | 2.24 | 84.12 | 2.64 | 85.68 | 26.67 | 10.95 |
| | 2.8 | | | | | | | | | |
| | EC | | | | | | | | | |
| | (2.8% | | | | | | | | | |
| | w/w) | | | | | | | | | |
| T 5 | Thiodicarb | 2 | gm | 8.24 | 2.63 | 81.36 | 2.78 | 84.92 | 26.12 | 9.07 |
| | 75% | | | | | | | | | |
| | WP | | | | | | | | | |
| T 6 | Spinosad | 0.5 | ml | 6.25 | 2.32 | 83.56 | 3.1 | 83.18 | 25.81 | 7.98 |
| | 480 | | | | | | | | | |
| | SC | | | | | | | | | |
| | (45% | | | | | | | | | |
| | w/w) | | | | | | | | | |
| T 7 | Chlorantraniliprole | 0.3 | ml | 7.25 | 2.74 | 80.58 | 2.97 | 83.88 | 25.88 | 8.23 |
| | 18.5% | | | | | | | | | |
| | SC | | | | | | | | | |
| T 8 | Spinetoram | 0.9 | ml | 9.18 | 2.55 | 81.93 | 3.11 | 83.13 | 25.8 | 7.95 |
| | 11.7% | | | | | | | | | |
| | SC | | | | | | | | | |
| T 9 | Emamectin | 0.4 | gm | 7.22 | 2.32 | 83.56 | 2.75 | 85.08 | 26.58 | 10.65 |
| | Benzonate | | | | | | | | | |
| | 5% SG | | | | | | | | | |
| T 10 | Chlorantraniliprole | 0.5 | ml | 6.28 | 2.65 | 81.22 | 3.58 | 80.58 | 25.61 | 7.26 |
| | (10%) + | | | | | | | | | |
| | Lambdacyhalothrin | | | | | | | | | |
| | (5%) | | | | | | | | | |
| | ZC | | | | | | | | | |
| T 11 | Cypermethrin | 1 | ml | 8.26 | 2.54 | 82.00 | 3.24 | 82.42 | 25.67 | 7.48 |
| | 25% | | | | | | | | | |
| | EC | | | | | | | | | |

| | | | | | | | | | |
|---|---|---|---|---|---|---|---|---|---|
| T 12 | Control | — | 10 | 14.11 | 0.00 | 18.43 | 0.00 | 23.75 | 0.00 |

| | | | | | | | |
|---|---|---|---|---|---|---|---|
| SE± | 1.52 | 1.69 | — | 1.72 | — | 1.59 | — |
| C.D. (0.05) | NS | 5.02 | — | 5.10 | — | 4.72 | — |

### Conclusion:

The results of the trial revealed that an application of Bio-Insecticide @ 2 ml/l recorded the maximum reduction in larval population of 92.84% over the control in 1^{st} spray and 92.84% reduction in larval population after 2^{nd} spray over the control.

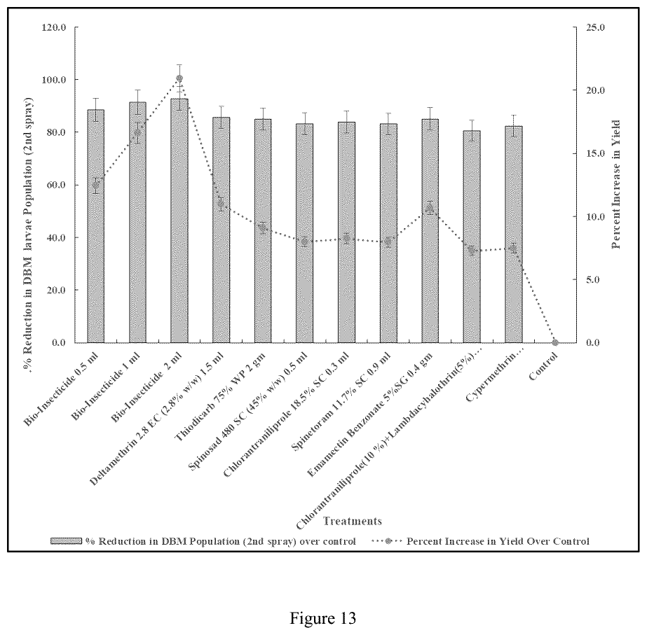

The bio stimulant effect of Bio-Insecticide with an application of 2 ml/l in Cauliflower exhibited the highest increase of 20.94% in yield over the control (FIG. 13).

### Example 21: Bio-Efficacy of Bio-Insecticide Against Mealy Bug in Grape

| | |
|---|---|
| Name of the Product: Bio-Insecticide | Variety: Sonaka |
| Crop: Grape | Pest: Mealy Bug |
| | (Pseudococcus maritimus) |

### Material and Method

A field experiment to study bio efficacy of Bio-Insecticide in Grape was conducted at Kay-Bee Bio-Organics Pvt. Ltd.'s farm at Phaltan, District Satara (M. S.), India, during 2021-2022. The trial consisting of twelve treatments including untreated control was laid out in a Randomized Block Design with three replications. Grape cultivated on following the standard spacing's 3 m×1.5 m to raise the good crop. Standard cultural practices were followed to grow a good crop. Observations on Mealy Bug population were recorded by randomly selected five plants in each treatment. Three bunches/plant were labelled to record Mealy Bug population. The data were analyzed by using standard statistical methods. Two sprays were taken pest population was recorded as per standard method in use before and after the application of Bio-Insecticide. The data were calculated by using standard statistical methods.

$$Reduction over control ( \% ) = \frac{\begin{matrix}Population Count of Control Plot - \\ Population Count of Treated Plot\end{matrix}}{Population Count of Control Plot} * 100$$

TABLE 23

| Tr. No. | Treatments | Dose ml or gm/L | |
|---|---|---|---|
| T 1 | Bio-Insecticide | 0.5 | ml |
| T 2 | Bio-Insecticide | 1 | ml |
| T 3 | Bio-Insecticide | 2 | ml |
| T 4 | Buprofezin 25% SC | 1.5 | ml |
| T 5 | Spirotetramat 15.31% OD | 1 | ml |
| T 6 | Spirotetramat 11.01% + | 1 | ml |
| | Imidacloprid 11.01% SC | | |
| T 7 | Fipronil 15% + Flonicamide 15% | 0.8 | gm |
| | WDG | | |
| T 8 | Chloropyriphos | 1 | ml |
| | 50% + Cypermethrin 5% EC | | |
| T 9 | Thiamethoxam 30% WG | 0.5 | gm |
| T 10 | Chloropyriphos 20% EC | 2 | ml |
| T 11 | Imidachloprid 70% WG | 0.3 | gm |

| | | |
|---|---|---|
| T 12 | Control | — |

### Results: The Results are Depicted in Table 24

TABLE 24

| | | | | | 1^{st} spray | | 2^{nd} spray | | | |
|---|---|---|---|---|---|---|---|---|---|---|
| | | | | | | % | | % | | |
| | | | | | No. of | Reduction | No. of | Reduction | | % |
| | | | | No. of | Mealy | in Mealy | Mealy | in Mealy | | Increase |
| | | | | Mealy | Bug | Bug | Bug | Bug | | in |
| | | | | Bug | after | Population | after | Population | | Yield |
| Tr. | | Dose ml | | before | 1st | (1st spray) | 2nd | (2nd spray) | Yield | over |
| No. | Treatments | or gm/L | | spray | spray | over control | spray | over control | (t/ha) | control |
| T 1 | Bio-Insecticide | 0.5 | ml | 32.46 | 6.65 | 84.38 | 6.12 | 86.31 | 17.02 | 15.28 |
| T 2 | Bio-Insecticide | 1 | ml | 38.02 | 5.56 | 86.94 | 4.12 | 90.78 | 17.52 | 17.69 |
| T 3 | Bio-Insecticide | 2 | ml | 36.07 | 4.46 | 89.52 | 2.45 | 94.52 | 18.12 | 20.42 |
| T 4 | Buprofezin 25% SC | 1.5 | ml | 33.45 | 7.71 | 81.88 | 6.10 | 86.35 | 15.89 | 9.25 |
| T 5 | Spirotetramat | 1 | ml | 37.56 | 8.16 | 80.83 | 7.12 | 84.07 | 16.02 | 9.99 |
| | 15.31% OD | | | | | | | | | |
| T 6 | Spirotetramat | 1 | ml | 35.12 | 7.96 | 81.30 | 6.13 | 86.28 | 15.69 | 8.09 |
| | 11.01% + | | | | | | | | | |
| | Imidacloprid 11.01% | | | | | | | | | |
| | SC | | | | | | | | | |
| T 7 | Fipronil 15% + | 0.8 | gm | 31.25 | 8.46 | 80.12 | 7.42 | 83.40 | 17.36 | 16.94 |
| | Flonicamide 15% | | | | | | | | | |
| | WDG | | | | | | | | | |
| T 8 | Chloropyriphos | 1 | ml | 35.26 | 9.45 | 77.80 | 7.79 | 82.57 | 17.03 | 15.33 |
| | 50% + Cypermethrin | | | | | | | | | |
| | 5% EC | | | | | | | | | |
| T 9 | Thiamethoxam 30% | 0.5 | gm | 36.78 | 9.39 | 77.94 | 6.89 | 84.58 | 16.89 | 14.62 |
| | WG | | | | | | | | | |
| T 10 | Chloropyriphos 20% | 2 | ml | 32.45 | 8.72 | 79.51 | 7.99 | 82.12 | 15.79 | 8.68 |
| | EC | | | | | | | | | |
| T 11 | Imidachloprid 70% | 0.3 | gm | 36.89 | 7.16 | 83.18 | 6.25 | 86.01 | 15.93 | 9.48 |

| | | | | | | | | | |
|---|---|---|---|---|---|---|---|---|---|
| | WG | | | | | | | | |
| T 12 | Control | — | 36.79 | 42.56 | — | 44.69 | — | 14.42 | — |

| | | | | | | | | |
|---|---|---|---|---|---|---|---|---|
| SE± | — | 1.43 | 1.81 | — | 1.56 | — | 1.91 | — |
| C.D. (0.05) | — | NS | 5.41 | — | 4.61 | — | 5.71 | — |

### Conclusion

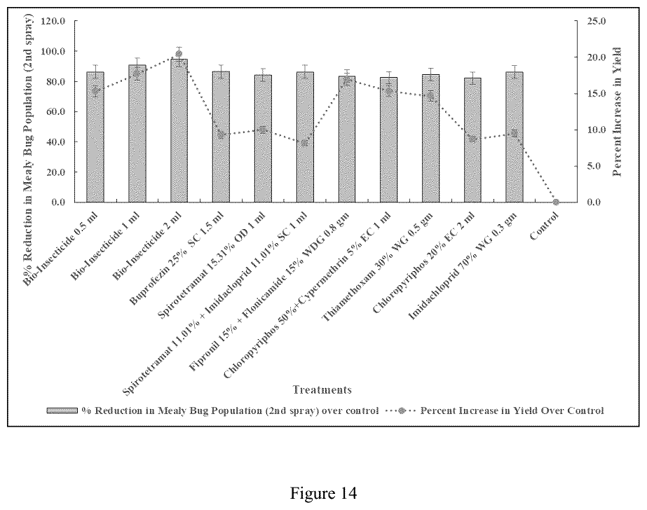

The results of the trial revealed that an application of Bio-Insecticide @ 2 ml/l recorded the maximum reduction in Mealy Bug population of 89.52% over the control in 1^{st} spray and 94.52% reduction in Mealy Bug population after 2^{nd} spray over the control. The bio stimulant effect of Bio-Insecticide with an application of 2 ml/l in Grape exhibited the highest increase of 20.42% in yield over the control (FIG. 14).

### Example 22: Bio-Efficacy of Bio-Insecticide Against Thrips in Onion

| | |
|---|---|
| Name of the Product: Bio-Insecticide | Variety: Baswant 780 |
| Crop: Onion | Pest: Thrips (Thrips tabaci) |

### Material and Method

A field experiment to study bio-efficacy of Bio-Insecticide in onion was conducted at Kay-Bee Bio-Organics Pvt. Ltd′.s farm at Phaltan, District Satara (M. S.), India during 2021-2022. The experiment comprising of twelve treatments was laid out in a Randomized Block Design (RBD) with three replications following spacings of 15×10 cm. Each treatment consisted of an area of 5×4 m. Recommended agronomic practices were followed raise the Onion crop. Observations on Thrips population were taken on randomly selected five onion plants. Two sprays were taken pest population was recorded as per standard method in use before and after the application of Bio-10 Insecticide. The data were calculated by using standard statistical methods.

$$Reduction over control ( \% ) = \frac{\begin{matrix}Population Count of Control Plot - \\ Population Count of Treated Plot\end{matrix}}{Population Count of Control Plot} * 100$$

TABLE 25

| Tr. | | | |
|---|---|---|---|
| No. | Treatments | Dose ml or gm/L | |
| T 1 | Bio-Insecticide | 0.5 | ml |
| T 2 | Bio-Insecticide | 1 | ml |
| T 3 | Bio-Insecticide | 2 | ml |
| T 4 | Fipronil 80% WG | 0.2 | gm |
| T 5 | Lambda Cyhalothrin 5% EC | 1 | ml |
| T 6 | Cyantraniliprole10.26% OD | 1 | ml |
| T 7 | Difenthiuron 50% WP | 1 | gm |
| T 8 | Imidachlopride 17.8% SL | 0.5 | ml |
| T 9 | Fluxametamide 10% EC | 0.8 | ml |
| T 10 | Profenophos 50% EC | 1 | ml |
| T 11 | Thimethoxam 25% WG | 0.5 | gm |

| | | |
|---|---|---|
| T 12 | Control | — |

### Results: The Results are Depicted in Table 26

TABLE 26

| | | | | | 1^{st} spray | | 2^{nd} spray | | | |
|---|---|---|---|---|---|---|---|---|---|---|
| | | | | | | % | | % | | |
| | | | | | | Reduction | | Reduction | | |
| | | | | | | in | | in | | |
| | | | | | | Thrips | | Thrips | | |
| | | | | | | Population | | Population | | % |
| | | | | No. of | No. of | (1^{st} | No. of | (2^{nd} | | Increase |
| | | Dose | | Thrips | Thrips | spray) | Thrips | spray) | | in Yield |
| Tr. | | ml or | | before | after 1^{st} | over | after 2^{nd} | over | Yield | over |
| No. | Treatments | gm/L | | spray | spray | control | spray | control | (t/ha) | control |
| T 1 | Bio- | 0.5 | ml | 22.18 | 2.98 | 87.08 | 3.04 | 88.26 | 39.99 | 14.33 |
| | Insecticide | | | | | | | | | |
| T 2 | Bio- | 1 | ml | 22.44 | 1.98 | 91.42 | 2.02 | 92.20 | 41.38 | 17.21 |
| | Insecticide | | | | | | | | | |
| T 3 | Bio- | 2 | ml | 22.63 | 1.48 | 93.58 | 1.32 | 94.90 | 42.54 | 19.46 |
| | Insecticide | | | | | | | | | |
| T 4 | Fipronil | 0.2 | gm | 21.92 | 3.63 | 84.27 | 3.72 | 85.64 | 37.78 | 9.32 |
| | 80% | | | | | | | | | |
| | WG | | | | | | | | | |
| T 5 | Lambda | 1 | ml | 22.83 | 3.15 | 86.35 | 3.48 | 86.56 | 39.52 | 13.31 |
| | Cyhalothrin | | | | | | | | | |
| | 5% | | | | | | | | | |
| | EC | | | | | | | | | |
| T 6 | Cyantraniliprole10.26% | 1 | ml | 22.76 | 4.07 | 82.36 | 4.28 | 83.47 | 36.89 | 7.13 |
| | OD | | | | | | | | | |
| T 7 | Difenthiuron | 1 | gm | 21.31 | 3.42 | 85.18 | 3.82 | 85.25 | 37.05 | 7.53 |
| | 50% | | | | | | | | | |
| | WP | | | | | | | | | |
| T 8 | Imidachlopride | 0.5 | ml | 22.43 | 4.18 | 81.88 | 4.31 | 83.36 | 36.66 | 6.55 |
| | 17.8% | | | | | | | | | |
| | SL | | | | | | | | | |
| T9 | Fluxametamide | 0.8 | ml | 22.78 | 3.21 | 86.09 | 3.41 | 86.83 | 38.02 | 9.89 |
| | 10% | | | | | | | | | |
| | EC | | | | | | | | | |
| T 10 | Profenophos | 1 | ml | 22.18 | 4.02 | 82.57 | 4.41 | 82.97 | 36.72 | 6.70 |
| | 50% | | | | | | | | | |
| | EC | | | | | | | | | |
| T 11 | Thimethoxam | 0.5 | gm | 22.37 | 3.70 | 83.96 | 3.91 | 84.90 | 37.18 | 7.85 |

| | | | | | | | | | |
|---|---|---|---|---|---|---|---|---|---|
| | 25% | | | | | | | | |
| | WG | | | | | | | | |
| T 12 | Control | — | 21.78 | 23.07 | 0.00 | 25.90 | 0.00 | 34.26 | — |

| | | | | | | | | |
|---|---|---|---|---|---|---|---|---|
| SE± | — | 0.53 | 1.26 | 1.82 | — | — | 2.02 | — |
| C.D. (0.05) | — | NS | 3.74 | 5.39 | — | — | 6.02 | — |

### Conclusion

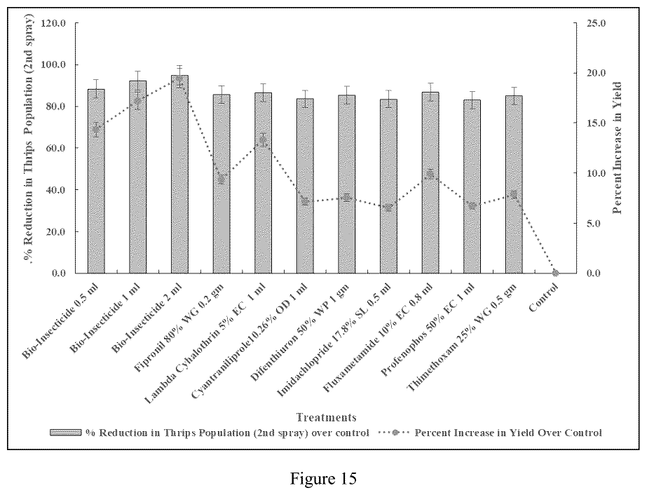

The results of the trial revealed that an application of Bio-Insecticide @ 2 ml/l recorded the maximum reduction in Thrips population of 93.58% over the control in 1st spay and 94.90% reduction in Thrips population after 2nd spray over the control. The bio stimulant effect of Bio-Insecticide with an application of 2 ml/l in Onion exhibited the highest increase of 19.46% in yield over the control (FIG. 15).

### Example 23: In Vitro Bio-Efficacy of Bio-Insecticide Against Diamond Back Moth in Cauliflower

| | |
|---|---|
| Name of the Product: Bio-Insecticide | Insect: Diamond Back Moth |
| Crop: Cauliflower | Scientific Name: Plutella xylostella |

### Material:

1. Specified Insect: Collected cauliflower Larval 3^{rd} instar DBM culture from insectary section
2. Host plant leaves: Cauliflower leaf is used for Diamond Back Moth feeding.
3. Spray gun: Required for spraying pesticides in control condition.
4. Solution for spray: 1 lit spray solution of respective pesticides was prepared.
5. Micropipette: Require for taking accurate volume of pesticide as per recommendations.
6. Microscope: Zoom stereo trinocular microscope for insect observation.

### Following Treatments was Used for Experiment

TABLE 27

| Treatments No. | Treatments | Dose ml or gm/L | |
|---|---|---|---|
| T 1 | Bio-Insecticide | 0.5 | ml |
| T 2 | Bio-Insecticide | 0.75 | ml |
| T 3 | Bio-Insecticide | 1 | ml |
| T 4 | Deltamethrin 2.8 EC (2.8% w/w) | 1.5 | ml |
| T 5 | Thiodicarb 75% WP | 2 | gm |
| T 6 | Spinosad 480 SC (45% w/w) | 0.5 | ml |
| T 7 | Chlorantraniliprole 18.5% SC | 0.3 | ml |
| T 8 | Spinetoram 11.7% SC | 0.9 | ml |
| T 9 | Emamectin Benzonate 5% SG | 0.4 | gm |
| T 10 | Chlorantraniliprole (10%) + | 0.5 | ml |
| | Lambdacyhalothrin(5%) ZC | | |
| T 11 | Cypermethrin 25% EC | 1 | ml |
| T 12 | Control | | |

### Method:—

### Direct Spray Method

Clean small cauliflower leaf was placed in a plastic petri plate. 10 Diamond back moth larvae per leaf per petri plate released which is taken from insect rearing unit. 12 treatments against 6 replications were maintained in experiment. Pesticide solution was prepared for 1 lit volume by using their respective recommended dosages ideal volume of pesticide per plastic petri plates was sprayed by using spray gun in control condition. Observations for mortality were taken at 48 hrs. and 72 hrs. after spray by using zoom stereoscope microscope. Calculation was done by standard statistical design CRD. Percent Corrected mortality was calculated by following Abbott formula:

$$\% Corrected Mortality = ( \% test mortality - \% control mortality / 100 - control mortality \times 100 ) .$$

### Results:—the Results are Shown in Table 28

TABLE 28

| | | | | Total no | | Total no of | | % |
|---|---|---|---|---|---|---|---|---|
| | | Dose | | of dead | % | dead | % | Corrected |
| Treatments | | ml or | | insects at | Mortality | insects at | Mortality | Mortality at |
| No. | Treatments | gm/L | | 48 hrs | at 48 hrs. | 72 hrs | at 72 hrs. | 72 hrs. |
| T 1 | Bio- | 0.5 | ml | 38 | 63.33 | 51 | 85.00 | 84.48 |
| | Insecticide | | | | | | | |
| T 2 | Bio- | 0.75 | ml | 46 | 76.67 | 56 | 93.33 | 93.10 |
| | Insecticide | | | | | | | |
| T 3 | Bio- | 1 | ml | 49 | 81.67 | 59 | 98.33 | 98.28 |
| | Insecticide | | | | | | | |
| T 4 | Deltamethrin | 1.5 | ml | 40 | 66.67 | 56 | 93.33 | 93.10 |
| | 2.8 EC | | | | | | | |
| | (2.8% w/w) | | | | | | | |
| T 5 | Thiodicarb | 2 | gm | 42 | 70.00 | 54 | 90.00 | 89.66 |
| | 75% WP | | | | | | | |
| T 6 | Spinosad | 0.5 | ml | 32 | 53.33 | 51 | 85.00 | 84.48 |
| | 480 SC | | | | | | | |
| | (45% w/w) | | | | | | | |
| T 7 | Chlorantraniliprole | 0.3 | ml | 40 | 66.67 | 52 | 86.67 | 86.21 |
| | 18.5% SC | | | | | | | |
| T 8 | Spinetoram | 0.9 | ml | 38 | 63.33 | 45 | 75.00 | 74.14 |
| | 11.7% SC | | | | | | | |
| T 9 | Emamectin | 0.4 | gm | 35 | 58.33 | 48 | 80.00 | 79.31 |
| | Benzonate | | | | | | | |
| | 5% SG | | | | | | | |
| T 10 | Chlorantraniliprole | 0.5 | ml | 36 | 60.00 | 50 | 83.33 | 82.76 |
| | (10%) + | | | | | | | |
| | Lambdacyhalothrin(5%) | | | | | | | |
| | ZC | | | | | | | |
| T 11 | Cypermethrin | 1 | ml | 39 | 65.00 | 51 | 85.00 | 84.48 |
| | 25% EC | | | | | | | |
| T 12 | Control | | | 0 | 0.00 | 2 | 3.33 | 0.00 |
| SE± | | | | 0.32 | | 0.46 | | |
| C.D. | | | | 1.64 | | 1.35 | | |
| (0.05) | | | | | | | | |

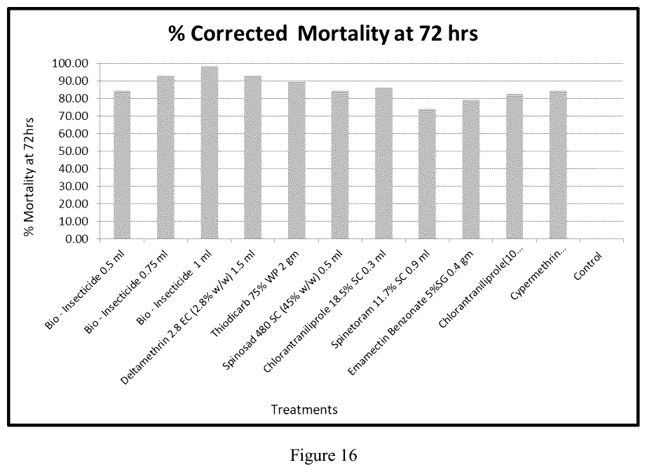

Conclusion: In vitro bio-efficacy study revealed that Bio-Insecticide @ 1 ml/lit showed highest mortality percentage 98.28% at 72 hours followed by Bio-Insecticide @ 0.75 ml/lit showed 93.10% against Diamond Back Moth (DBM) (FIG. 16).

### Example 24: In Vitro Bio-Efficacy of Bio-Insecticide Against Mealy Bug in Grapes

| | |
|---|---|
| Name of the Product: Bio-Insecticide | Insect: Mealy Bug |
| Crop: Grapes | Scientific Name: |
| | Pseudococcus maritimus |

### Material:

1. Specified Insect: Collected grapes mealy bug culture from insectary section
2. Host plant leaves: Grapes leaf is used for mealy bug feeding.
3. Spray gun: Required for spraying pesticides in control condition.
4. Solution for spray: 1 lit spray solution of respective pesticides was prepared.
5. Micropipette: Require for taking accurate volume of pesticide as per recommendations.
6. Microscope: Zoom stereo trinocular microscope for insect observation.

### Following Treatments was Used for Experiment

TABLE 29

| Treatments No. | Treatments | Dose ml or gm/L | |
|---|---|---|---|
| T 1 | Bio-Insecticide | 0.5 | ml |
| T 2 | Bio-Insecticide | 1 | ml |
| T 3 | Bio-Insecticide | 1.5 | ml |
| T 4 | Buprofezin 25% SC | 1.5 | ml |
| T 5 | Spirotetramat 15.31% OD | 1 | ml |
| T 6 | Spirotetramat 11.01% + | 1 | ml |
| | Imidacloprid 11.01% SC | | |
| T 7 | Fipronil 15% + Flonicamide | 0.8 | gm |
| | 15% WDG | | |
| T 8 | Chloropyriphos | 1 | ml |
| | 50% + Cypermethrin 5% EC | | |
| T 9 | Thiamethoxam 30% WG | 0.5 | gm |
| T 10 | Chloropyriphos 20% EC | 2 | ml |
| T 11 | Imidachloprid 70% WG | 0.3 | gm |

| | | |
|---|---|---|
| T 12 | Control | — |

### Method:—

### Direct Spray Method

Clean small grapes leaf was placed in a petri plate. 10 Mealy bug per leaf per petri plate released which is taken from insect rearing unit. 12 treatments against 6 replications were maintained in experiment. Pesticide solution was prepared for 1 lit volume by using their respective recommended dosages ideal volume of pesticide per petri plate was sprayed by using spray gun in control condition. Observations for mortality were taken at 24 hrs and 48 hrs. After spray by using zoom stereoscope microscope. Calculation was done by standard statistical design CRD. Percent corrected mortality was calculated by following Abbott formula:

$$\% Corrected Mortality = ( \% test mortality - \% control mortality / 100 - control mortality \times 100 ) .$$

### Results: The Results are Depicted in Table 30

TABLE 30

| | | | | Total no | | Total no | | % |
|---|---|---|---|---|---|---|---|---|
| | | | | of dead | % | of dead | % | Corrected |
| Treatments | | Dose ml | | insects at | Mortality | insects | Mortality | Mortality |
| No. | Treatments | or gm/L | | 24 hrs | at 24 hrs | at48 hrs | at 48 hrs | at 48 hrs |
| T 1 | Bio-Insecticide | 0.5 | ml | 44 | 73.33 | 50 | 83.33 | 83.33 |
| T 2 | Bio-Insecticide | 1 | ml | 48 | 80.00 | 54 | 90.00 | 90.00 |
| T 3 | Bio-Insecticide | 1.5 | ml | 52 | 86.67 | 58 | 96.67 | 96.67 |
| T 4 | Buprofezin 25% | 1.5 | ml | 30 | 50.00 | 40 | 66.67 | 66.67 |
| | SC | | | | | | | |
| T 5 | Spirotetramat | 1 | ml | 32 | 53.33 | 35 | 58.33 | 58.33 |
| | 15.31% OD | | | | | | | |
| T 6 | Spirotetramat | 1 | ml | 25 | 41.67 | 32 | 53.33 | 53.33 |
| | 11.01% + | | | | | | | |
| | Imidacloprid | | | | | | | |
| | 11.01% SC | | | | | | | |
| T 7 | Fipronil 15% + | 0.8 | gm | 15 | 25.00 | 40 | 66.67 | 66.67 |
| | Flonicamide 15% | | | | | | | |
| | WDG | | | | | | | |
| T 8 | Chloropyriphos | 1 | ml | 20 | 33.33 | 38 | 63.33 | 63.33 |
| | 50% + Cypermethrin | | | | | | | |
| | 5% EC | | | | | | | |
| T 9 | Thiamethoxam | 0.5 | gm | 22 | 36.67 | 32 | 53.33 | 53.33 |
| | 30% WG | | | | | | | |
| T 10 | Chloropyriphos | 2 | ml | 12 | 20.00 | 30 | 50.00 | 50.00 |
| | 20% EC | | | | | | | |
| T 11 | Imidachloprid | 0.3 | gm | 22 | 36.67 | 35 | 58.33 | 58.33 |
| | 70% WG | | | | | | | |

| | | | | | | | |
|---|---|---|---|---|---|---|---|
| T 12 | Control | — | 0 | 0.00 | 0 | 0.00 | 0.00 |

| | | |
|---|---|---|
| SE± | 0.48 | 0.38 |
| C.D. (0.05) | 1.38 | 0.93 |

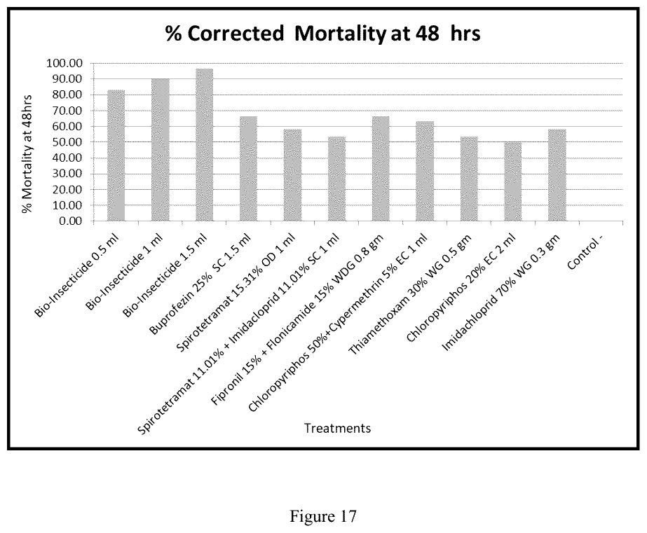

Conclusion: Invitro bio-efficacy study revealed that Bio-Insecticide @ 1.5 ml/lit showed highest mortality percentage 96.67% at 48 hours followed by Bio-Insecticide @ 1 ml/lit showed 90.00% against grapes mealy bug (FIG. 17).

### Example 25: In Vitro Bio-Efficacy of Bio-Insecticide Against Thrips in Onion

| | |
|---|---|
| Name of the Product: Bio-Insecticide | Insect: Thrips |
| Crop: Onion | Scientific Name: Thrips tabaci |

### Material:

1. Specified Insect: Collected onion nymphal stage thrips culture from insectary section
2. Host plant leaves: Onion tender leaf is used for thrips feeding.
3. Spray gun: Required for Spraying pesticides in control condition.
4. Solution for spray: 1 lit spray solution of respective pesticides was prepared.
5. Micropipette: Require for taking accurate volume of pesticide as per recommendations.
6. Microscope: Zoom stereo trinocular microscope for insect observation.

### Following Treatments was Used for Experiment

TABLE 31

| Treatments No. | Treatments | Dose ml or gm/L | |
|---|---|---|---|
| T 1 | Bio-Insecticide | 0.5 | ml |
| T 2 | Bio-Insecticide | 1 | ml |
| T 3 | Bio-Insecticide | 1.5 | ml |
| T 4 | Fipronil 80% WG | 0.2 | gm |
| T 5 | Lambda Cyhalothrin 5% EC | 1 | ml |
| T 6 | Cyantraniliprole10.26% OD | 1 | ml |
| T 7 | Difenthiuron 50% WP | 1 | gm |
| T 8 | Imidachlopride 17.8% SL | 0.5 | ml |
| T 9 | Fluxametamide 10% EC | 0.8 | ml |
| T 10 | Profenophos 50% EC | 1 | ml |
| T 11 | Thimethoxam 25% WG | 0.5 | gm |
| T 12 | Control | | |

### Method:—

### Direct Spray Method

Clean small onion tender leaf was placed in a petri plate. 10 thrips per leaf per petri plate released which is taken from insect rearing unit. 12 treatments against 6 replications were maintained in experiment. Pesticide solution was prepared for 1 lit volume by using their respective recommended dosages ideal volume of pesticide per Petri plate was sprayed by using spray gun in control condition. Observations for mortality were taken at 24 hrs and 48 hrs. After spray by using zoom stereoscope microscope. Calculation was done by standard statistical design CRD. Percent Corrected mortality was calculated by following Abbott formula:

$$\% Corrected Mortality = ( \% test mortality - \% control mortality / 100 - control mortality \times 100 )$$

### Results: The Results are Depicted in Table 32

TABLE 32

| | | | | | | % |
|---|---|---|---|---|---|---|
| | | | | Total no of | % | Corrected |
| Treatments | | Dose ml | | dead insects | Mortality | Mortality |
| No. | Treatments | or gm/L | | at48 hrs | at 48 hrs | at 48 hrs |
| T 1 | Bio-Insecticide | 0.5 | ml | 47 | 78.33 | 76.79 |
| T 2 | Bio-Insecticide | 1 | ml | 53 | 88.33 | 87.50 |
| T 3 | Bio-Insecticide | 1.5 | ml | 58 | 96.67 | 96.43 |
| T 4 | Fipronil 80% WG | 0.2 | gm | 50 | 83.33 | 82.14 |
| T 5 | Lambda Cyhalothrin | 1 | ml | 48 | 80.00 | 78.57 |
| | 5% EC | | | | | |
| T 6 | Cyantraniliprole10.26% | 1 | ml | 40 | 66.67 | 64.29 |
| | OD | | | | | |
| T 7 | Difenthiuron 50% WP | 1 | gm | 45 | 75.00 | 73.21 |
| T 8 | Imidachlopride 17.8% | 0.5 | ml | 50 | 83.33 | 82.14 |
| | SL | | | | | |
| T 9 | Fluxametamide 10% | 0.8 | ml | 47 | 78.33 | 76.79 |
| | EC | | | | | |
| T 10 | Profenophos 50% EC | 1 | ml | 46 | 76.67 | 75.00 |
| T 11 | Thimethoxam 25% WG | 0.5 | gm | 49 | 81.67 | 80.36 |
| T 12 | Control | | | 4 | 6.67 | 0.00 |
| | SE± | | | 0.36 | | |
| | C.D. (0.05) | | | 1.10 | | |

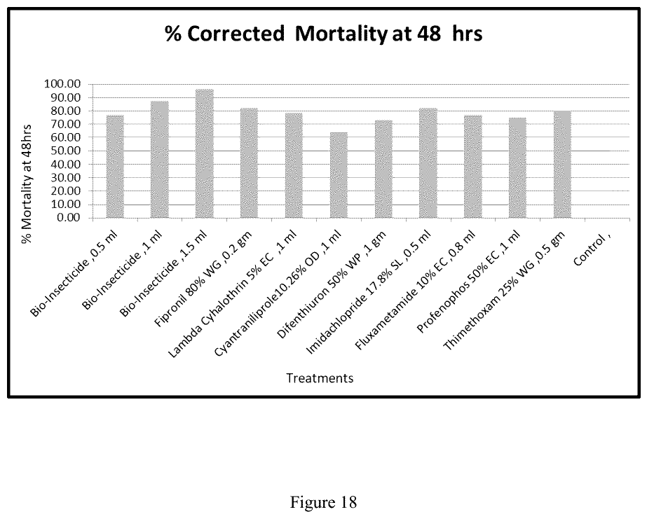

Conclusion: Invitro bio-efficacy study revealed that Bio-Insecticide @ 1.5 ml/lit showed highest mortality percentage 96.43% at 48 hours followed by Bio-Insecticide @ 1 ml/lit showed 87.50% against onion thrips (FIG. 18).

1. A synergistic botanical based bio-insecticidal, bio-stimulant composition for controlling pests, comprising:
i. Thymus vulgaris extract or Enriched and Purified extract of Thymus vulgaris or Purified Phyto ingredients or marker compounds such as 1,8-cineole, camphor, α-pinene, camphene, carvarcrol and thymolin extracted from said botanical source at a concentration in a range of 0.001-35%;
ii. Cinnamomum cassia extract or Enriched and Purified extract of Cinnamomum cassia or Purified Phyto ingredients or marker compounds such as Cinnamic aldehyde, catechin, epicatechin, quercitrin, protocatechuic acid and cinnamic acid extracted from said botanical source at a concentration in a range of 0.001-35%;
iii. Cuminum cyminum extract or Enriched and Purified extract of Cuminum cyminum or Purified Phyto ingredients or marker compounds such as Cumin aldehyde, alkaloid, anthraquinone, saponin, tannin and steroid extracted from said botanical source at a concentration in a range of 0.001-45%;
iv. Abrus precatorius extract or enriched and purified extract of Abrus precatorius or Purified Phyto ingredients or marker compounds such as Abrin and α-tocopherol extracted from said botanical source at a concentration in a range of 0.001-45%;
v. Piper nigrum extract or Enriched and Purified extract of Piper nigrum or Purified Phyto ingredients or marker compounds such as Piperine, amides, piperidine and pyrrolidines extracted from said botanical source at a concentration in a range of 0.001-35%;
vi. Piper longum extract or Enriched and Purified extract of Piper longum or Purified Phyto ingredients or marker compounds such as methyl piperine, Piperine piperlongumine, piperlonguminine, pipercide and piperidine extracted from said botanical source at a concentration in a range of 0.001-45%;
vii. Syzygium aromaticum extract or Enriched and Purified extract of Syzygium aromaticum or Purified Phyto ingredients or marker compounds such as Eugenyl acetate, eugenol, and β-caryophyllene extracted from said botanical source at a concentration in a range of 0.001-45%;
viii. Pongamia pinnata extract or Enriched and Purified extract of Pongamia pinnata or Purified Phyto ingredients or marker compounds such as karanjin, glabrin, and kanjone extracted from said botanical source at a concentration in a range of 0.001-45%;
ix. Anethum graveolens extract or Enriched and Purified extract of Anethum graveolens or Purified Phyto ingredients or marker compounds such as alkaloids, flavonoids, tannins and saponins extracted from said botanical source at a concentration in a range of 0.001-45%;
x. Cinnamomum camphora extract or Enriched and Purified extract of Cinnamomum camphora or Purified Phyto ingredients or marker compounds such as linalool, 1,8-cineole, α-terpineol, and camphor extracted from said botanical source at a concentration in a range of 0.001-45%;
xi. Acorus calamus extract or Enriched and Purified extract of Acorus calamus or Purified Phyto ingredients or marker compounds such as methyl ethers, camphene, P cymene, a selinene, bgurjunene, β cadinene, camphor, shyobunones, linalool and preisocalamendiol extracted from said botanical source at a concentration in a range of 0.001-45%;
xii. Citrus limon extract or Enriched and Purified extract of Citrus limon or Purified Phyto ingredients or marker compounds such as Pinene (β), Limonene, Terpinene (γ), Terpinen-4-ol, Neral Geranial extracted from said botanical source at a concentration in a range of 0.001-45%;
xiii. Azadirachta indica extract or Enriched and Purified extract of Azadirachta indica or Purified Phyto ingredients or marker compounds such as Annonacin, Squamocin, Annonin, kaempferol-3-O and lupeol, β- and α-amyrin, quercetin, camphor, ricin, epicatechin, catechin, linoleic acid and ricinoleic acid extracted from said botanical source at a concentration in a range of 0.001-45%;
xiv. Sesamum indica extract or Enriched and Purified extract of Sesamum indica or Purified Phyto ingredients or marker compounds such as Sesamin, kaempferol-3-O and lupeol, β- and α-amyrin, quercetin, camphor, ricin, epicatechin, catechin, linoleic acid and ricinoleic acid extracted from said botanical source at a concentration in a range of 0.001-55%;
xv. Argemone mexicana extract or Enriched and Purified extract of Argemone Mexicana or Purified Phyto ingredients or marker compounds such as berberine, cryptopine, scoulerine, stylopine, sanguinarine, thalifoline and protopine extracted from said botanical source at a concentration in a range of 0.001-55%;
xvi. Moringa oleifera extract or Enriched and Purified extract of Moringa oleifera or Purified Phyto ingredients or marker compounds such as alkaloids, saponins, tannins, steroids, phenolic acids, glucosinolates, flavonoids, and terpenes extracted from said botanical source at a concentration in a range of 0.001-55%;
xvii. Brassica juncea extract or Enriched and Purified extract of Brassica juncea or Purified Phyto ingredients or marker compounds such as brassinolide, polyphenols, phenolic acids, flavonoids, carotenoids (zeaxanthin, lutein, β-carotene), alkaloids, tannins and saponins extracted from said botanical source at a concentration in a range of 0.001-55%;
xviii. Lantana camara extract or Enriched and Purified extract of Lantana camara or Purified Phyto ingredients or marker compounds such as tannin, saponin, flavonoids, terpenoids, alkaloids, carbohydrate, anthroquinone and polyphenols extracted from said botanical source at a concentration in a range of 0.001-55%;
xix. Datura stramonium extract or Enriched and Purified extract of Datura stramonium or Purified Phyto ingredients or marker compounds such as Atropine, scopolamine and hyoscyamine extracted from said botanical source at a concentration in a range of 0.001-55%;
xx. Melia dubia extract or Enriched and Purified extract of Melia dubia or Purified Phyto ingredients or marker compounds such as Alkaloids, Carbohydrate, Phenol, Saponin, Terpenoids, Coumarin and Glycoside extracted from said botanical source at a concentration in a range of 0.001-55%;
xxi. Murraya koenigii extract or Enriched and Purified extract of Murraya koenigii or Purified Phyto ingredients or marker compounds such as Alkaloids, Carbohydrate, Phenol, Saponin, Terpenoids, Coumarin and Glycoside extracted from said botanical source at a concentration in a range of 0.001-55%;
xxii. Ipomoea carnea extract or Enriched and Purified extract of Ipomoea carnea or Purified Phyto ingredients or marker compounds such as 2-ethyl-1,3-dimethylbenzene, 2-(12-pentadecynyloxy) tetrahydro2H-pyran, 3-furanyl [2-hydroxy-4-methyl-2-(2-methylpropyl)cyclopentyl]-methanone, 2,2-dideuterooctadecanal and hexadecanoic acid extracted from said botanical source at a concentration in a range of 0.001-55%;
xxiii. Ocimum sanctum extract or Enriched and Purified extract of Ocimum sanctum or Purified Phyto ingredients or marker compounds such as oleanolic acid, rosmarinic acid, ursolic acid, eugenol, methyl eugenol, linalool, carvacrol, β elemene and β caryophyllene extracted from said botanical source at a concentration in a range of 0.001-55%;
xxiv. Jatropha integerrima extract or Enriched and Purified extract of Jatropha integerrima or Purified Phyto ingredients or marker compounds such as Anthocyanin, coumarin, glycoside, phenol, saponin, and tannin extracted from said botanical source at a concentration in a range of 0.001-55%;
xxv. Acacia concinna extract or Enriched and Purified extract of Acacia concinna or Purified Phyto ingredients or marker compounds such as lupeol, spinasterol, acacic acid, lactone and natural sugars glucose extracted from said botanical source at a concentration in a range of 0.001-55%;
xxvi. Mentha piperita extract or Enriched and Purified extract of Mentha piperita or Purified Phyto ingredients or marker compounds such as lupeol, spinasterol, acacic acid, lactone and natural sugars glucose extracted from said botanical source at a concentration in a range of 0.001-55%;
xxvii. Coriandrum sativum extract or Enriched and Purified extract of Coriandrum sativum or Purified Phyto ingredients or marker compounds such as Linalool, neryl acetate, γ-terpinene and α-pinene extracted from said botanical source at a concentration in a range of 0.001-55%;
xxviii. Annona squamosa extract or Enriched and Purified extract of Annona squamosa or Purified Phyto ingredients or marker compounds such as Annonacin, Squamocin, Annonin extracted from said botanical source at a concentration in a range of 0.001-45%;
xxix. Organic emulsifier at a concentration in a range of 0.001-20%;
xxx. Ionic emulsifier at a concentration in a range of 0.001-7%;
xxxi. Non-ionic emulsifier at a concentration in a range of 0.001-10%; and
xxxii. Carrier oils at a concentration in a range of 0.001 to 90%.

2. The synergistic composition, as claimed in claim 1, wherein said Organic emulsifier is in a range of 10-12%.

3. The synergistic composition, as claimed in claim 1, wherein said Ionic emulsifier is in a range of 4-6%.

4. The synergistic composition, as claimed in claim 1, wherein said Non-ionic emulsifiers is in a range of 5-7%.

5. The synergistic composition, as claimed in claim 1, wherein said Carrier oils is in a range of 60-65%.

6. The synergistic composition, as claimed in claim 1, wherein said organic emulsifier is selected from the group comprising rice bran wax, cetearyl glucoside, aloevera extract, plant saponins, aqueous extract of Indian soapberry (sapindus mukorassi), aqueous extract of Acacia concinna, hydrolyzed coconut oil, bee wax, gaur gum and the like.

7. The synergistic composition, as claimed in claim 1, wherein said Ionic and non-ionic emulsifier are selected from the group comprising aqueous extract of Indian soapberry (sapindus mukorassi), aqueous extract of Acacia concinna, hydrolyzed coconut oil, bee wax, gaur gum gaur gum, poly sorbate 20, poly sorbate 60, poly sorbate 80, Lecithin, soy lecithin, sodium phosphates, monoglycerides, diglycerides, sodium stearoyl lactylate, glyceryl oleate, ethoxylated oleate, ethoxylated soybean oil, ethoxylated castor oil and the like.

8. The synergistic composition as claimed in claim 1, wherein said carrier oils are selected from the group comprising seed oil of Anethum graveolens, Pongamaia pinnata oil, neem oil, castor oil, fish oil, sesame oil, mustard oil, canola oil, or water.

9. The synergistic composition, as claimed in claim 1, wherein said composition has a particle size in the range of 10 to 900 nanometer.

10. The synergistic composition, as claimed in claim 9, wherein the particle size is in the range of 10 to 100 nm.

11. The synergistic composition, as claimed in claim 1, wherein said composition comprises:
i. Melia dubia extract in the range of 0.001 to 35%;
ii. Cinnamomum cassia extract in the range of 0.001 to 35%;
iii. Piper longum extract in the range of 0.001 to 40%;
iv. Lantana camara extract in the range of 0.001 to 25%;
v. Murraya koenigii extract in the range of 0.001 to 35%;
vi. Acorus calamus extract in the range of 0.001 to 35%;
vii. Ipomoea carnea extract in the range of 0.001 to 25%;
viii. Gaur gum in the range of 0.001 to 18%;
ix. ethoxylated castor oil in the range of 0.001 to 20%; and
x. Canola oil in the range of 0.001 to 90%.

12. The synergistic composition, as claimed in claim 1, wherein said composition comprises:
i. Mentha piperita extract in the range of 0.001 to 35%;
ii. Piper nigrum extract in the range of 0.001 to 35%;
iii. Acorus calamus extract in the range of 0.001 to 40%;
iv. Coriandrum sativum extract in the range of 0.001 to 25%;
v. Cinnamomum cassia extract in the range of 0.001 to 35%;
vi. Gaur gum in the range of 0.001 to 18%;
vii. ethoxylated castor oil in the range of 0.001 to 20%; and
viii. Canola oil in the range of 0.001 to 90%.

13. The synergistic composition, as claimed in claim 1, wherein said composition comprises:
i. Piper longum extract in the range of 0.001 to 35%;
ii. Syzygium aromaticum extract in the range of 0.001 to 35%;
iii. Mentha piperita extract in the range of 0.001 to 40%;
iv. Coriandrum sativum extract in the range of 0.001 to 25%;
v. Acorus calamus extract in the range of 0.001 to 35%;
vi. Gaur gum in the range of 0.001 to 18%;
vii. ethoxylated castor oil in the range of 0.001 to 20%; and
viii. Canola oil in the range of 0.001 to 90%.

14. The synergistic composition, as claimed in claim 1, wherein said composition comprises:
i. Argemone mexicana extract in the range of 0.001 to 35%;
ii. Datura stramonium extract in the range of 0.001 to 35%;
iii. Piper longum extract in the range of 0.001 to 40%;
iv. Lantana camara extract in the range of 0.001 to 25%;
v. Brassica juncea extract in the range of 0.001 to 35%;
vi. Acorus calamus extract in the range of 0.001 to 35%;
vii. Ipomoea carnea extract in the range of 0.001 to 25%;
viii. Gaur gum in the range of 0.001 to 18%;
ix. ethoxylated castor oil in the range of 0.001 to 20%; and
x. Canola oil in the range of 0.001 to 90%.

15. The synergistic composition, as claimed in claim 1, wherein said composition comprises:
i. Lantana camara extract in the range of 0.001 to 35%;
ii. Piper nigrum extract in the range of 0.001 to 35%;
iii. Cinnamomum camphora extract in the range of 0.001 to 40%;
iv. Piper longum extract in the range of 0.001 to 25%;
v. Ocimum santum extract in the range of 0.001 to 35%;
vi. Thymus vulgaris extract in the range of 0.001 to 35%;
vii. Citrus limon extract in the range of 0.001 to 25%;
viii. Gaur gum in the range of 0.001 to 18%;
ix. ethoxylated castor oil in the range of 0.001 to 20%; and
x. Canola oil in the range of 0.001 to 90%.

16. The synergistic composition, as claimed in claim 1, wherein said composition comprises:
i. Abrus precatorius extract in the range of 0.001 to 35%;
ii. Acorus calamus extract in the range of 0.001 to 35%;
iii. Jatropha integerrima extract in the range of 0.001 to 40%;
iv. Piper longum extract in the range of 0.001 to 25%;
v. Acacia concinna extract in the range of 0.001 to 35%;
vi. Cuminum cyminum extract in the range of 0.001 to 35%;
vii. Gaur gum in the range of 0.001 to 18%;
viii. ethoxylated castor oil in the range of 0.001 to 20%; and
ix. Canola oil in the range of 0.001 to 90%.

17. A process for preparation of synergistic composition as claimed in claim 1 comprising:
a. Extracting 0.001-35% of Thymus vulgaris or purified phyto ingredients or marker compounds such as 1,8-cineole, camphor, α-pinene, camphene, carvarcrol and thymolin extracted from said botanical source; 0.001-35% of Cinnamomum cassia or Purified Phyto ingredients or marker compounds such as Cinnamic aldehyde, catechin, epicatechin, quercitrin, protocatechuic acid and cinnamic acid extracted from said botanical source; 0.001-45% of Cuminum cyminum or Purified Phyto ingredients or marker compounds such as Cumin aldehyde, alkaloid, anthraquinone, saponin, tannin and steroid extracted from said botanical source; 0.001-45% of Abrus precatorius or Purified Phyto ingredients or marker compounds such as Abrin and α-tocopherol extracted from said botanical source; 0.001-35% of Piper nigrum or Purified Phyto ingredients or marker compounds such as Piperine, amides, piperidine and pyrrolidines extracted from said botanical source; 0.001-45% of Piper longum or Purified Phyto ingredients or marker compounds such as methyl piperine, Piperine piperlongumine, piperlonguminine, pipercide and piperidine extracted from said botanical source; 0.001-45% of Syzygium aromaticum or Purified Phyto ingredients or marker compounds such as Eugenyl acetate, eugenol, and β-caryophyllene extracted from said botanical source; 0.001-45% of Pongamia pinnata Purified Phyto ingredients or marker compounds such as karanjin, glabrin, and kanjone extracted from said botanical source; 0.001-45% of Anethum graveolens or Purified Phyto ingredients or marker compounds such as alkaloids, flavonoids, tannins and saponins extracted from said botanical source; 0.001-45% of Cinnamomum camphora or Purified Phyto ingredients or marker compounds such as linalool, 1,8-cineole, α-terpineol, and camphor extracted from said botanical source; 0.001-45% of Acorus calamus or Purified Phyto ingredients or marker compounds such as methyl ethers, camphene, P cymene, a selinene, bgurjunene, β cadinene, camphor, shyobunones, linalool and preisocalamendiol extracted from said botanical source; 0.001-45% of Citrus limon or Purified Phyto ingredients or marker compounds such as Pinene (β), Limonene, Terpinene (γ), Terpinen-4-ol, Neral Geranial extracted from said botanical source; 0.001-45% of Azadirachta indica or Purified Phyto ingredients or marker compounds such as Annonacin, Squamocin, Annonin, kaempferol-3-O and lupeol, β- and α-amyrin, quercetin, camphor, ricin, epicatechin, catechin, linoleic acid and ricinoleic acid extracted from said botanical source; 0.001-55% of Sesamum indica or Purified Phyto ingredients or marker compounds such as Sesamin, kaempferol-3-O and lupeol, β- and α-amyrin, quercetin, camphor, ricin, epicatechin, catechin, linoleic acid and ricinoleic acid extracted from said botanical source; 0.001-55% of Argemone Mexicana or Purified Phyto ingredients or marker compounds such as berberine, cryptopine, scoulerine, stylopine, sanguinarine, thalifoline and protopine extracted from said botanical source; 0.001-55% of Moringa oleifera or Purified Phyto ingredients or marker compounds such as alkaloids, saponins, tannins, steroids, phenolic acids, glucosinolates, flavonoids, and terpenes extracted from said botanical source; 0.001-55% of Brassica juncea or Purified Phyto ingredients or marker compounds such as brassinolide, polyphenols, phenolic acids, flavonoids, carotenoids (zeaxanthin, lutein, β-carotene), alkaloids, tannins and saponins extracted from said botanical source; 0.001-55% of Lantana camara or Purified Phyto ingredients or marker compounds such as tannin, saponin, flavonoids, terpenoids, alkaloids, carbohydrate, anthroquinone and polyphenols extracted from said botanical source; 0.001-55% of Datura stramonium or Purified Phyto ingredients or marker compounds such as Atropine, scopolamine and hyoscyamine extracted from said botanical source; 0.001-55% of Melia dubia or Purified Phyto ingredients or marker compounds such as Alkaloids, Carbohydrate, Phenol, Saponin, Terpenoids, Coumarin and Glycoside extracted from said botanical source; 0.001-55% of Murraya koenigii or Purified Phyto ingredients or marker compounds such as Alkaloids, Carbohydrate, Phenol, Saponin, Terpenoids, Coumarin and Glycoside extracted from said botanical source; 0.001-55% of Ipomoea carnea or Purified Phyto ingredients or marker compounds such as 2-ethyl-1,3-dimethylbenzene, 2-(12-pentadecynyloxy) tetrahydro2H-pyran, 3-furanyl [2-hydroxy-4-methyl-2-(2-methylpropyl)cyclopentyl]-methanone, 2,2-dideuterooctadecanal and hexadecanoic acid extracted from said botanical source; 0.001-55% of Ocimum sanctum or Purified Phyto ingredients or marker compounds such as oleanolic acid, rosmarinic acid, ursolic acid, eugenol, methyl eugenol, linalool, carvacrol, β elemene and β caryophyllene extracted from said botanical source; 0.001-55% of Jatropha integerrima or Purified Phyto ingredients or marker compounds such as Anthocyanin, coumarin, glycoside, phenol, saponin, and tannin extracted from said botanical source; 0.001-55 of Acacia concinna or Purified Phyto ingredients or marker compounds such as lupeol, spinasterol, acacic acid, lactone and natural sugars glucose extracted from said botanical source; 0.001-55% of Mentha piperita or Purified Phyto ingredients or marker compounds such as lupeol, spinasterol, acacic acid, lactone and natural sugars glucose extracted from said botanical source; 0.001-55% of Coriandrum sativum or Purified Phyto ingredients or marker compounds such as Linalool, neryl acetate, γ-terpinene and α-pinene extracted from said botanical source; 0.001-45% of Annona squamosa or Purified Phyto ingredients or marker compounds such as Annonacin, Squamocin, Annonin extracted from said botanical source;
b. mixing the obtained extracts at given proportion along with 0.001-20% of organic emulsifier, preferably 10-12%; 0.001-7% of Ionic, preferably 4-6%; and 0.001-10% of non-ionic emulsifiers, preferably 5-7%;
c. blending the mixture obtained in step (b) with 0.001 to 90% of carrier oils, preferably 60-65%;
d. stirring the mixture obtained in step (c) at 300-1000 RPM, preferably 500-800 RPM at 25 to 55° C., preferably 35° C. in close mixing vessel for continued stirring followed by homogenizing the mix with the homogenizer fitted towards the bottom of vessel and with the speed of 3700˜27000 rpm to reduce the particle size;
e. passing the homogenized mixture obtained in step (d) through in line shear pump with 1800 RPM to 5800 RPM to reduce particle size to obtain the nano emulsion with particle size ranging from 10 to 900 nano meter;
f. passing the mixture obtained in step (e) through High-pressure homogenization to reduce the particle size below 100 nano meters;
g. passing the mixture obtained in step (f) through sparkler filter having seven layers of membrane filters with pore size less than 1-2 micron to achieve desired filtration; and
h. recovering the final product.

18. The process as claimed in claim 17, wherein extraction process of step (a) comprises enrichment and purification of the extract using techniques which include but not limited to liquid-liquid extraction based on polarity of phyto ingredient, column chromatography, precipitation and crystallisation to obtain purified phyto ingredients.

19. The process as claimed in claim 17, wherein high-pressure homogenization of step (f) is carried out at speed of 4000 rpm to 10000 rpm and pressure up to 4,200 bar (60,000 psi).

20. The process as claimed in claim 17, wherein said extraction techniques include but not limited to hydrophilic extraction, super critical carbon dioxide extraction, steam distillation, solvent extraction and Soxhlet extraction.

21. The process as claimed in claim 20, wherein said extraction technique is Solvent extraction and/or super critical carbon dioxide extraction

22. The process as claimed in claim 17, wherein said solvent used in the solvent extraction includes but not limited to methanol, hexane, acetone, ethyl acetate, butanol, ethanol and the like.

23. The process as claimed in claim 22, wherein said solvent used in the solvent extraction is methanol, ethyl acetate, hexane alone or mixtures thereof.

24. The process as claimed in claim 17, wherein said organic emulsifier include but not limited to rice bran wax, cetearyl glucoside, aloevera extract, plant saponins, aqueous extract of Indian soapberry (sapindus mukorassi), aqueous extract of Acacia concinna, hydrolyzed coconut oil, bee wax, gaur gum and the like.

25. The process as claimed in claim 17, wherein said Ionic and non-ionic emulsifier include but not limited, aqueous extract of Indian soapberry (sapindus mukorassi), aqueous extract of Acacia concinna, hydrolyzed coconut oil, bee wax, gaur gum gaur gum, poly sorbate 20, poly sorbate 60, poly sorbate 80, Lecithin, soy lecithin, sodium phosphates, monoglycerides, diglycerides, sodium stearoyl lactylate, glyceryl oleate, ethoxylated oleate, ethoxylated soybean oil, ethoxylated castor oil.

26. The synergistic composition, as claimed in claim 1, wherein said composition comprises combination of standardised enriched botanical extract and purified phyto ingredient.

27. The synergistic composition, in claim 1, wherein the Phyto ingredients include saponins, polyphenols, alkaloids, flavonoids, terpenoids, or secondary metabolites.

28. The synergistic composition, in claim 1, wherein the composition may be in the form of liquid, paste, powder or powder converted into granule or tablet form and the like.

29. The synergistic composition, as claimed in claim 28, wherein the composition may be in liquid form.

30. The synergistic composition, in claim 1, wherein said composition may be applied by sprinkler application, sprayer application, drip application and the like.

31. The synergistic composition, in claim 1, for use in controlling the pests and improving yield of crop plants.

32. The synergistic composition, as claimed in claim 27, wherein said composition is effective against major agricultural pests such as non-flying insects, caterpillar, larvae, sucking pest likes white fly, hopper, Jassid, mealy bugs, aphids, spider mites, slugs, thrips and the like.

33. The synergistic composition, in claim 1, wherein said composition exhibits biostimulant activity and thereby improves plant growth and crop yield.

34. A method for controlling the pest and promoting the growth of the plants comprising applying the bio-insecticidal composition as claimed in claim 1 to the plant or to diseased affected parts of the plant in an effective amount.
